(19) United States
(12) Patent Application Publication
(10) Pub. No.: US 2004/0072170 A1
(43) Pub. Date: Apr. 15, 2004

(54) Novel target genes for diseases of the heart

(76) Inventors: Daniela Beck nee Bunk, Iffeldorf (DE); Birgit Reuner, Munich (DE); Joachim Beck, Iffeldorf (DE); Thomas Henkel, Kassel (DE)

Correspondence Address: CLARK & ELBING LLP, 101 FEDERAL STREET, BOSTON, MA, 02110, US

(21) Appl. No.: 10/276,775

(22) PCT Filed: May 30, 2001
(86) PCT No.: PCT/EP01/06165

(51) Int. Cl. C12Q 1/68

(52) U.S. Cl. 435/6

(57) ABSTRACT

The present invention relates to a variety of genes abnormally expressed in heart tissue as well as to fragments of such genes. Assessment of the expression level of these genes may be used for testing the predisposition of mammals and preferably humans for a heart disease or for an acute state of such a disease. Preferred diseases in accordance with the invention are congestive heart failure, dilative cardiomyopathy, hypertrophic cardiomyopathy and ischemic cardiomyopathy. The present invention further relates to methods of identifying compounds capable of normalizing the expression level of the aforementioned genes and of further genes affected by the abnormal expression. The identified compounds may be used for formulating compositions, preferably pharmaceutical compositions for preventing or treating diseases. They may also be used as lead compounds for the development of medicaments having an improved efficiency, a longer half-life, a decreased toxicity etc. and to be employed in the treatment of heart diseases. Included in the invention are also somatic gene therapy methods comprising the introduction of at least one functional copy of any of the above-mentioned genes into a suitable cell. Finally, the invention relates to non-human transgenic animals comprising at least one of the aforementioned genes in their germ line. The transgenic animals of the invention may be used for the development of medicaments for the treatment of heart diseases.

A variety of documents is cited throughout this specification. The disclosure content of said documents is herewith incorporated by reference.

The present invention is based on the finding that a variety of genes is abnormally expressed in diseased heart tissue. Assessment of the expression level of these genes may be used for testing the predisposition of mammals and preferably humans for a heart disease or for an acute state of such a disease. Diseases that preferably relate to the present invention are congestive heart failure, dilative cardiomyopathy, hypertrophic cardiomyopathy and ischemic cardiomyopathy. The present invention further relates to methods of identifying compounds capable of normalizing the expression level of the aforementioned genes and of further genes affected by the abnormal expression. The identified compounds may be used for formulating compositions, preferably pharmaceutical compositions, for preventing or treating diseases. They may also be used as lead compounds for the development of medicaments having an improved efficiency, a longer half-life, a decreased toxicity etc. and to be employed in the treatment of heart diseases. Included in the invention are also somatic gene therapy methods comprising the introduction of at least one functional copy of any of the above-mentioned genes into a suitable cell. Finally, the invention relates to non-human transgenic animals comprising at least one of the aforementioned genes in their germ line. The transgenic animals of the invention may be used for the development of medicaments for the treatment of heart diseases.

Referring to studies of the American Heart Association, about 60 million people in the USA suffer from Cardiovascular diseases like high blood pressure (50.0 mio), Coronary heart disease (12.4 mio), Myocardial infarction (7.3 mio), Angina pectoris (6.4 mio), Stroke (4.5 mio), Congenital cardiovascular defects (1.0 mio), and Congestive heart failure (4.7 mio). Hence, it follows that 20 percent of whole population is affected. The mortality was 949,619 in 1998 in the USA, which means that about 40% of all deaths were caused by Cardiovascular diseases. Since 1900 Cardiovascular diseases are the number one cause of death (1918 was an exception) with one death every 33 seconds on average. At present there is no causal treatment for congestive heart failure available.

Accordingly, the technical problem underlying the present invention was to provide a new generation of tools useful in the diagnosis, prevention and treatment of heart-related diseases.

The solution to said technical problem is achieved by providing the methods of independent claims 1, 3, 12, 13, 15, 19, 21, 22, 23, 27, 29, 31, 32, 34, 35, 36, 40 to 44, and 46, the monoclonal antibody according to claim 14, the transgenic non-human mammal according to claim 16, and the use according to independent claim 47. Further advantageous features, aspects and details of the invention are evident from the dependent claims, the description, the examples and the drawings.

The invention is based upon the unexpected result that the certain genes coding for the protein sequences given in examples 2 to 11 are deregulated in the comparison of one or more failing heart samples to one or more non-filing heart samples and lead to an upregulation (examples 2, 5, 8, 9, 10) or downregulation (examples 3, 4, 6, 7) of the described polypeptides measured by their respective mRNAs or cDNAs. The significant changes in gene expression levels suggest a causative role in congestive heart failure.

However, such a causative role for one specific indication of the heart leads to the assumption that a deregulation of such gene(s) might play an important role in other diseases of the heart as well. Such involvement can easily be tested by methods well known in the art and described e.g. in example 1 of the present application by a comparison of the gene expression levels of such gene between a sample of a healthy mammal and of a mammal having the disease in question. Therefore the subject of this invention does not only relate to dilated cardiomyopathy but also to other diseases of the heart.

It is well accepted in the art that upregulation of gene expression of a downregulated target gene by means of a gene therapeutic intervention, compensatory molecules or specific activators, for example of transcription or translation, are potentially very promising therapeutic tools to treat a heart disease that is caused or promoted by the downregulation of such gene.

On the other hand, downregulation of gene expression and/or protein function of an upregulated target gene by means of specific inhibitors, antisense constructs, ribozyms, antibodies or any other compound (as hereinafter definded) are well accepted tools to treat a heart disease that is caused or promoted by the upregulation of such gene.

As one gene might be upregulated for one indication of the heart whereas the same gene might be downregulated for another indication of the heart, both upregulation of gene expression as well as downregulation of gene expression and/or protein function might be useful for the same target gene in different indications.

The same holds true for methods for identifying a subject at risk for a disease of the heart, a method for identifying a compound, a method for identifying one or a plurality of genes as well as methods to make transgenic non-human mammals. In all these various embodiments of the invention aberrant gene expression in either direction can be used for the given methods.

Thus, the present invention relates to a method for identifying a subject at risk for a disease of the heart, comprising the step of quantitating in the heart tissue of the subject the amount of at least one RNA encoding an amino acid sequence selected from the group consisting of:

(a) the amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequ nce of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];

(b) an amino acid sequence that is at least 60%, preferably at least 80%, especially at least 90%, advantageously at least 99% identical to the amino acid sequence of (a);

(c) the amino acid sequence of (a) with at least one conservative amino acid substitution;

(d) an amino acid sequence that is an isoform of the amino acid sequence of any of (a) to (c);

(e) the RNA transcribed from the DNA sequence of SEQ ID NO: 10 [NM_{—}003970], the DNA sequence of SEQ ID NO: 11 [AW755252], the DNA sequence of SEQ ID NO: 12 [EST clone 52706], the DNA sequence of SEQ ID NO: 13 [EST clone 56461], the DNA sequence of SEQ ID NO: 14 [M14780], the DNA sequence of SEQ ID NO: 15 [61166contig], the DNA sequence of SEQ ID NO: 16 [AF161698], the DNA sequence of SEQ ID NO: 17 [65330contig], the DNA sequence of SEQ ID NO: 18 [66214cds] or the DNA sequence AF129505, or the DNA sequence of SEQ ID NO: 19 [X83703] or a degenerate variant thereof; and

(f) an amino acid that is encoded by a DNA molecule the complementary strand of which hybridizes in 4×SSC, 0.1% SDS at 65° C. to the DNA molecule encoding the amino acid sequence of (a), (c) or (d).

The term “disease of the heart” means, in accordance with the present invention, any disease that affects the normal function of the heart. This definition includes hereditary as well as acquired diseases such as diseases induced by a pathogen or diseases due to lack of exercise.

Several diseases of the heart are, for example, rheumatic fever/rheumatic heart disease, hypertensive heart disease, hypertensive heart and renal disease, ischemic heart disease (coronary heart disease), diseases of pulmonary circulation (which include acute and chronic pulmonary heart disease), arrhythmias, congenital heart disease, angina and congestive heart failure.

The term “quantitating the amount of at least one RNA” is intended to mean the determination of the amount of mRNA in heart tissue as compared to a standard value such as an internal standard. The (internal) standard would advantageously be the amount of a corresponding RNA produced by a heart tissue not affected by a disease. Said (internal) standard would also include a mean value obtained from a variety of heart tissues not affected by a disease. A possible way to get samples of heart tissue would be to take a biopsy (catheter) from the ventricular wall. Optionally, a standard would take into account the genetic background of the subject under investigation. Thus, quantitation of said subject's RNA is effected in comparison to the amount of RNA of one or a variety of samples of the same or a similar genetic background. A variable number of “non-failing” humans (humans that do not show an indication for any heart disease) are compared with a variable number of patients that suffer a distinct heart disease like dilated cardiomyopathy. The determination can be effected by any known technology of analysing the amount of RNA produced in a sample such as a tissue sample. Techniques based on hybridisation like Northern-Blot, dot-blot, subtractive hybridisation, DNA-Chip analysis or techniques based on reverse transcription coupled to the polymerase chain reaction (RT-PCR) like differential display, suppression subtractive hybridisation (SSH), fluorescence differential display (FDD), serial analysis of gene expression (SAGE) or representational difference analysis (see e.g. Kozian, D. H., Kirschbaum, B. J.; Comparative gene-expression analysis. (1999) 17:73-77). Generally, it is preferred that the assay is performed as a high throughput assay. This holds also true for the further methods described herein and in accordance with this invention. Samples of RNA may be prepared as described in the appended examples.

The term “isoform” means a derivative of a gene resulting from alternative splicing, alternative polyadenylation, alternative promoter usage or RNA editing. Isoforms can be detected by

(a) in silico analysis (e.g. by clustering analysis of any types of expressed sequences or the corresponding proteins, by alignment of expressed sequences with chromosomal DNA, by interspecies comparisons or by analysis of the coding as well as non-coding sequences like promoters or regulatory RNA processing sites for SNPs or known mutations causing a disease).

(b) any type of hybridisation techniques (1,2) (e.g. Northern blots, nuclease protection assays, microarrays) starting from RNA.

(c) PCR-applications as well as hybridisation techniques starting from single strand or double strand cDNA obtained by reverse transcription (3), as described for example in Higgins, S. J., Hames, D. RNA Processing: A practical approach Oxford University Press (1994), Vol. 1 and 2; Sambrook, Fritsch, Maniatis, Molecular Cloning, a laboratory manual. (1989) Cold Spring Harbor Laboratory Press; Stoss, O. Stoilov, P., Hartmann, A. M., Nayler, O., Stamm, S., The in vivo minigene approach to analyse tissue-specific splicing. Brain Res. Brain Res. Protoc. (1999), 3:383-394.

Primers/probes for RT-PCR or hybridisation techniques are designed in a fashion that at least one of the primers/probes recognizes specifically one isoform. If differences in the molecular weight of isoforms are big enough to separate them with electrophoretical or chromatographical methods, it is also possible to detect multiple isoforms at once by employing primers/probes that flank the spliced regions. The isoforms are then sequenced and analysed as described in (a).

The term “DNA molecule the complementary strand of which hybridizes in 4×SSC, 0.1% SDS at 65° C. to the DNA molecule encoding the amino acid sequence of (a), (c) or (d)” means that the two DNA molecules hybridize under these experimental conditions to each other. This term does not exclude that the two DNA sequences hybridize at higher stringency conditions such as 2×SSC, 0.1% SDS at 65° C. nor does it exclude that lower stringency conditions such as 6×SSC. 0.1% SDS at 60° C. allow a hybridization of the two DNA sequences.

Appropriate hybridization conditions for each sequence may be established on well-known parameters such as temperature, composition of the nucleic acid molecules, salt conditions etc.; see, for example, Sambrook et al., “Molecular Cloning, A Laboratory Manual”; CSH Press, Cold Spring Harbor, 1989 or Higgins and Harnes (eds.), “Nucleic acid hybridization, a practical approach”, IRL Press, Oxford 1985, see in particular the chapter “Hybridization Strategy” by Britten & Davidson, 3 to 15.

In accordance with the present invention it has surprisingly been found that a variety of genes is aberrantly expressed in diseases associated with the heart and in particular in patients suffering from congestive heart failure. By performing the method of the invention which may be in vivo, in vitro or in silico, the diagnosis of a disease of the heart established by a different methodology may be corroborated. Alternatively, it may be assessed whether a subject that is preferably throughout this specification a human displaying no sign of being affected by a disease of the heart is at risk of developing such a disease. This is possible in cases where the aberrant expression of the gene defined herein above is causative of the disease or is a member of a protein cascade wherein another gene/protein than the one identified herein above is causative for said disease. In this regard, the term “causative” is not limited to mean that the aberrant expression of one gene as identified above or which is a member of said protein cascade is the sole cause for the onset of the disease. Whereas this option is also within the scope of the invention, expression the invention also encompasses embodiments wherein said aberrant is one of a variety of causative events that lead to the onset of the disease.

There is causal correlation between altered cellular function of cardiomyocytes and its protein composition. The latter is regulated by three main mechanisms:

a. Gene expression

b. Alternative splicing

c. Posttranslational modification

In a variation of the method of the invention quantitation of the above recited RNA is used to monitor the progress of a disease of the heart (said variation also applies to the method described herein below). This variation may be employed for assessing the efficacy of a medicament or to determine a time point when administration of a drug is no longer necessary or when the dose of a drug may be reduced and/or when the time interval between administrations of the medicament may be increased. This variation of the method of the invention may successfully be employed in cases where an aberrant expression of any of the aforementioned genes/genes as members of protein cascades is causative of the disease. It is also useful in cases where the aberrant expression of the gene/genes is the direct or indirect result of said disease.

When assessing the risk or the status of the disease, one or more of the RNA levels may be determined. Generally, the assessment of more than 1, such as 2, 3, 4, 5, 6, 7, 8, 9 or 10 different RNAs is expected to enhance the fidelity of the prognosis/diagnosis. However, the gain in fidelity would, as a rule, have to be weighted against the costs generated by such additional tests. Accordingly, it is preferred that one or two different RNA levels are determined for a first assessment. If deemed necessary or appropriate, further RNA levels may be determined.

In a preferred embodiment of the method of the invention the amount of the said RNA is quantitated using a nucleic acid probe which is a nucleic acid comprising a sequence selected from the group consisting of:

(a) the DNA sequence of SEQ ID NO: 10 [NM_{—}003970], the DNA sequence of SEQ ID NO: 11 [AW755252], the DNA sequence of SEQ ID NO: 12 [EST clone 52706], the DNA sequence of SEQ ID NO: 13 [EST clone 56461], the DNA sequence of SEQ ID NO: 14 [M14780], the DNA sequence of SEQ ID NO: 15 [61166contig], the DNA sequence of SEQ ID NO: 16 [AF161698], the DNA sequence of SEQ ID NO: 17 [65330contig], the DNA sequence of SEQ ID NO: 18 [66214cds] or the DNA sequence AF129505, or the DNA sequence of SEQ ID NO: 19 [X83703] or a degenerate variant thereof (b) a DNA sequence at least 60%, preferably 80%, especially 90%, advantageously 99% identical to the DNA sequence of (a); (c) a nucleic acid sequence that encodes the amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; with at least one conservative amino acid substitution; (d) a nucleic acid sequence that encodes an amino acid sequence that is at least 60%, preferably 80%, especially 90%, advantageously 99% identical to the amino acid sequence of (b); (e) a nucleic acid sequence that encodes the amino acid sequence of (a) or (b) with at least one conservative amino acid substitution; (f) a nucleic acid sequence that hybridizes in 4×SSC, 0.1% SDS at 65° C. to the complementary strand of the DNA molecule encoding the amino acid sequence of (a) or (c); and (g) a fragment of at least 15 nucleotides in length of (a) to (f).

Advantageously, the nucleic acid sequence which is preferably a DNA sequence is detectably labeled. Appropriate labels include radioactive labels, wherein the radioactivity conferring molecules may be, e.g., ³²P, ³⁵S or ³H. Appropriate labels further include fluorescent, phosphorescent or bioluminescent labels or nucleic acid sequences coupled to biotin or streptavidin in order to detect them via anti-biotin or anti-streptavidin antibodies. Whereas any of the above mentioned probes specifically hybridizing to the aforementioned RNAs may be employed, it is preferred that fragments of the full length coding sequence such as oligomers of a length between 15 and 25 nucleotides are used. Examples of such oligomers are oligomers of 18, 21 or 24 nucleotides. Alternatively, the double strand formed after hybridization can be detected by anti-double strand DNA specific antibodies or aptamers etc.

In this regard, it is understood that the probe of SEQ ID NO: 10 and the mentioned variants thereof are used for quantitating the RNA of SEQ ID NO: 1, but not to any of the other mentioned RNAs. In the following, appropriate pairs of RNAs and corresponding probes for assessing risks etc. of diseases of the heart are mentioned with the understanding that (i) appropriate variants of the probes as mentioned above may be used and (ii) said probes are specific for the corresponding RNA only but not for any of the other mentioned RNAs. These pairs are: SEQ ID NOs: 2/SEQ ID NO: 11; SEQ ID NO: 3/SEQ ID NO: 13; SEQ ID NO: 4/SEQ ID NO: 14; SEQ ID NO: 5/SEQ ID NO: 15; SEQ ID NO: 6/SEQ ID NO: 16; SEQ ID NO: 7/SEQ ID NO: 17; SEQ ID NOs: 8/SEQ ID NO: 18; SEQ ID NO: 9/SEQ ID NO: 19.

After hybridization, appropriate washing steps are performed in order to remove unspecific signals. Appropriate washing conditions include 2 wash steps at 65° C. with 2×SSC, 0.1% SDS for 30 min (50 ml) and finally two wash steps with 50 ml of a solution containing 0.1×SSC, 0.1% SDS for 30 min.; see also Sambrook et al., Ioc. cit., Higgins and Hames, Ioc. cit. After washing, the label is detected, depending on its nature. For example, a radioactive label may be detected by exposure to an X-ray film or by a phosphorimager. Alternatively, biotinylated probes can be detected by fluorescence, e.g. by using SAPE (streptavidin-phycoerythrin) with subsequent detection of the signal by a laser scanner.

In addition, the invention relates to a method for identifying a subject at risk for a disease of the heart, comprising the step of quantitating in the heart tissue of the subject the amount of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution. Further included are polypeptides encoded by any of the above recited nucleic acid sequences. This holds also true for any of the other embodiments in which the aforementioned polypeptides are employed.

This embodiment of the invention makes use of the option that detection may not only be at the level of the mRNA but also at the level of the polypeptide translated from the mRNA. Whereas it is not excluded that the level of mRNA strictly correlates with the level of polypeptide translated from the mRNA, this may not always be the case. Accordingly, it may be assessed whether the mRNA or the protein level, if different, is more appropriate to establish if the heart of a subject is prone to develop a disease of the heart. Factors that contribute to differences in the expression levels of mRNA and protein are well-known in the art and include differential mRNA-export to the protein-synthesis machinery as well as differences in the translation efficacy of different mRNA species. Other considerations influencing the choice of the detection level (in RNA or protein) include the availability of an appropriate screening tool, instrumentation of the lab, experience of the lab personnel and others.

In a preferred embodiment of the method of the invention, the amount of the said polypeptide is quantitated using an antibody that specifically binds a polypeptide selected from the group consisting of: (a) the polypeptide having amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably 80%, especially 90%, advantageously 99% identical to the amino acid sequence of (a); and (c) a polypeptide having the amino acid sequence of (a) with at least one conservative amino acid substitution, or an antigen-binding portion of said antibody.

The antibody used in accordance with the invention may be a monoclonal or a polyclonal antibody (see Harlow and Lane, Antibodies, A Laboratory Manual”, CSH Press, Cold Spring Harbor, USA, 1988) or a derivative of said antibody which retains or essentially retains its binding specificity. Whereas particularly preferred embodiments of said derivatives are specified further herein below, other preferred derivatives of such antibodies are chimeric antibodies comprising, for example, a mouse or rat variable region and a human constant region. The term “specifically binds” in connection with the antibody used in accordance with the present invention means that the antibody etc. does not or essentially does not cross-react with (poly)peptides of similar structures. Cross-reactivity of a panel of antibodies etc. under investigation may be tested, for example, by assessing binding of said panel of antibodies etc. under conventional conditions (see, e.g., Harlow and Lane, Ioc. cit.) to the polypeptide of interest as well as to a number of more or less (structurally and/or functionally) closely related polypeptides. Only those antibodies that bind to the polypeptide of interest but do not or do not essentially bind to any of the other (poly)peptides which are preferably expressed by the same tissue as the polypeptide of interest, i.e. heart, are considered specific for the polypeptide of interest and selected for further studies in accordance with the method of the invention.

In a particularly preferred embodiment of the method of the invention, said antibody or antibody binding portion is or is derived from a human antibody or a humanized antibody.

The term “humanized antibody” means, in accordance with the present invention, an antibody of non-human origin, where at least one complementarity determining region (CDR) in the variable regions such as the CDR3 and preferably all 6 CDRs have been replaced by CDRs of an antibody of human origin having a desired specificity. Optionally, the non-human constant region(s) of the antibody has/have been replaced by (a) constant region(s) of a human antibody. Methods for the production of humanized antibodies are described in, e.g., EP-A1 0 239 400 and WO90/07861.

The specifically binding antibody etc. may be detected by using, for example, a labeled secondary antibody specifically recognizing the constant region of the first antibody. However, in a further particularly preferred embodiment of the method of the invention, the antibody, the binding portion or derivative thereof itself is detectably labeled.

Detectable labels include a variety of established labels such as radioactive (125I, for example) or fluorescent labels (see, e.g. Harlow and Lane, Ioc. cit.). Binding may be detected after removing unspecific labels by appropriate washing conditions (see, e.g. Harlow and Lane, Ioc. cit.).

In an additionally preferred embodiment of the method of the invention, said derivative of said antibody is an scFv fragment.

The term “scFv fragment” (single-chain Fv fragment) is well understood in the art and preferred due to its small size and the possibility to recombinantly produce such fragments.

In a preferred embodiment of the method of the invention, said RNA is obtained from heart tissue.

A suitable way would be to take a biopsy (catheter) from the ventricular wall. The decision to do this is clearly affected by the severity of the disease and the general constitution of the patient. The cardiologist and the patient have to drive the final decision. In an additionally preferred embodiment of the method of the invention, said polypeptide is quantitated in heart tissue.

In another preferred embodiment, the method of the invention further comprises the step of normalizing the amount of RNA against a corresponding RNA from a healthy subject or cells derived from a healthy subject.

The term “healthy subject” means a subject without any indication for heart disease.

The term “normalizing the amount of RNA against a corresponding RNA from a healthy subject or cells derived from a healthy subject” means, in accordance with the present invention, that levels of mRNA from a comparative number of cells from the heart of said subject under investigation and from the heart of an individual not affected by a disease of the heart are compared. Alternatively, cells from the heart of the subject under investigation may be compared in terms of the indicated mRNA levels with cells derived from the heart of a healthy individual which are kept in cell culture and optionally form a cell line. Optionally, different sources of cells such as from different individuals and/or different cell lines may be used for the generation of the standard against which the mRNA level of the subject under investigation is compared.

Using the Affymetrix Chip technology, there is also the possibility to use external standards (that are given separately to the hybridisation cocktail) in order to normalize the values of different oligonucleotide-chips.

In yet another preferred embodiment, the method of the invention further comprises the step of normalizing the amount of polypeptide against a corresponding polypeptide from a healthy subject or cells derived from a healthy subject.

The same considerations as developed for the previous embodiment on the mRNA level apply here to the normalization of protein levels.

Additionally, the invention relates to a method for identifying a compound that increases or decreases the level in heart tissue of a polypeptide selected from the group consisting of: (a) the polypeptide having amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino; acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, comprising the steps of: (1) contacting a DNA encoding said polypeptide under conditions that would permit the translation of said polypeptide with a test compound; and (2) detecting an increased or decreased level of the polypeptide relative to the level of translation obtained in the absence of the test compound.

The term “compound” shall mean any biologically active substance that has an effect on heart tissue or a single heart cell, whereas such compound has a positive or negative influence upon such heart tissue or heart cell. Preferred compounds are nucleic acids, preferably coding for a peptide, polypeptide, antisense RNA or a ribozyme or nucleic acids that act independent from their transcription respective their translation as for example as an antisense RNA or ribozyme; natural or synthetic peptids preferably with a relative molecular mass of about 1.000, especially of about 500 peptide analogs polypeptides or compositions of polypeptides, proteins, protein complexes, fusion proteins, preferably antibodies, especially murine, human or humanized antibodies, single chain antibodies, F_{ab} fragments or any other antigen binding portion or derivative of an antibody, including modifications of such molecules as for example glycosylation, acetylation, phosphorylation, farnesylation, hydroxylation, methylation or estrification hormones, organic or anorganic molecules or compositions, preferably small molecules with a relative molecular mass of about 1.000, especially of about 500.

The term “under conditions that would permit the translation of said polypeptide” denotes any conditions that allow the in vitro or in vivo translation of the polypeptide of interest. As regards in vitro conditions, translation may be effected in a cell-free system, as described, for example in Stoss, Schwaiger, Cooper and Stamm (1999). J. Biol. Chem. 274: 10951-10962), using the TNT-coupled reticulocyte lysate system (Promega) With respect to in vivo conditions, physiological conditions such as conditions naturally occurring inside a cell are preferred.

Based on the finding that expression of genes encoding the above recited polypeptides is aberrant, the method of the invention allows the convenient identification or isolation of compounds that counteract such aberrant expression such that normal expression levels are restored or essentially restored.

The DNA encoding the polypeptide of interest would normally be contained in an expression vector. The expression vectors may particularly be plasmids, cosmids, viruses or bacteriophages used conventionally in genetic engineering that comprise the aforementioned polynucleotide. Preferably, said vector is a gene transfer or targeting vector. Expression vectors derived from viruses such as retroviruses, vaccinia virus, adeno-associated virus, herpes viruses, or bovine papilloma virus, may be used for delivery of the polynucleotides into targeted cell population. Methods which are well known to those skilled in the art can be used to construct recombinant viral vectors; see, for example, the techniques described in Sambrook et al., Molecular Cloning A Laboratory Manual, Cold Spring Harbor Laboratory (1989) N.Y. and Ausubel et al., Current Protocols in Molecular Biology, Green Publishing Associates and Wiley Interscience, N.Y. (1989). Alternatively, the polynucleotides and vectors can be reconstituted into liposomes for delivery to target cells. The vectors containing the polynucleotides can be transferred into the host cell by well-known methods, which vary depending on the type of cellular host. For example, calcium phosphate or DEAE-Dextran mediated transfection or electroporation may be used for eukaryotic cellular hosts; see Sambrook, supra.

Such vectors may comprise further genes such as marker genes which allow for the selection of said vector in a suitable host cell and under suitable conditions. The polynucleotide is operatively linked to expression control sequences allowing expression in eukaryotic cells. Expression of said polynucleotide comprises transcription of the polynucleotide into a translatable mRNA. Regulatory elements ensuring expression in eukaryotic cells, preferably mammalian cells, are well known to those skilled in the art. They usually comprise regulatory sequences ensuring initiation of transcription and, optionally, a poly-A signal ensuring termination of transcription and stabilization of the transcript, and/or an intron further enhancing expression of said polynucleotide. Additional regulatory elements may include transcriptional as well as translational enhancers, and/or naturally-associated or heterologous promoter regions. Possible regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the polynucleotide. Furthermore, depending on the expression system used leader sequences capable of directing the polypeptide to a cellular compartment or secreting it into the medium may be added to the coding sequence of the aforementioned polynucleotide and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDV1 (Pharmacia), pCDM8, pRc/CMV, pcDNA1, pcDNA3, the Echo™ Cloning System (Invitrogen), pSPORT1 (GIBCO BRL) or pRevTet-On/pRevTet-Off or pCI (Promega).

Preferably, the expression control sequences will be eukaryotic promoter systems in vectors capable of transforming or transfecting eukaryotic host cells. As mentioned above, the vector used in the method of the present invention may also be a gene transfer or targeting vector. Gene therapy, which is based on introducing therapeutic genes into cells by ex-vivo or in-vivo techniques, is one of the most important applications of gene transfer. Suitable vectors and methods for in-vitro or in-vivo gene therapy are described in the literature and are known to the person skilled in the art; see, e.g., Giordano, Nature Medicine 2 (1996), 534-539; Schaper, Circ. Res. 79 (1996), 911-919; Anderson, Science 256 (1992), 808-813; Isner, Lancet 348 (1996), 370-374; Muhlhauser, Circ. Res. 77 (1995), 1077-1086; Wang, Nature Medicine 2 (1996), 714-716; WO94/29469; WO 97/00957 or Schaper, Current Opinion in Biotechnology 7 (1996), 635-640, and references cited therein. The polynucleotides and vectors may be designed for direct introduction or for introduction via liposomes, or viral vectors (e.g. adenoviral, retroviral) into the cell. Preferably, said cell is a germ line cell, embryonic cell, or egg cell or derived therefrom, most preferably said cell is a stem cell.

The vector comprising the DNA would be used to transform a suitable eukaryotic host cell. Upon expression of the DNA, which may be constitutive or induced, the test compound would be contacted with the DNA. This can be done by introducing the test compound into the cell. For example, if the test compound is a (poly)peptide, then introduction may be effected by transfection of the corresponding DNA, optionally comprised in a suitable expression vector. If the compound is a small molecule, preferably with a relative molecular weight of up to 1,000, especially up to 500, the introduction into the cell may be effected by direct administration, plus DMSO for hydrophobic compounds, probably liposomal transfer.

In the case that the method of the invention is carried out in vitro, for example, in a cell-free system, then introduction into a cell would not be necessary. Rather, the test compound would be admixed to the in vitro expression system and the effect of said admixture observed.

The effect of the contact of the DNA of interest with the test compound on the protein level may be assessed by any technology that measures changes in the quantitative protein level. Such technologies include Western blots, ELISAs, RIAs and other techniques referred to herein above.

The change in protein level, if any, as a result of the contact of said DNA and said test compound is compared against a standard. This standard is measured applying the same test system but omits the step of contacting the compound with the DNA. The standard may consist of the expression level of the polypeptide after no compound has been added. Alternatively, the DNA may be contacted with a compound that has previously been demonstrated to have an influence on the expression level.

Compounds tested positive for being capable of enhancing or reducing the amount of polypeptide produced are prime candidates for the direct use as a medicament or as lead compounds for the development of a medicament. Naturally, the toxicity of the compound identified and other well-known factors crucial for the applicability of the compound as a medicament will have to be tested. Methods for developing a suitable active ingredient of a pharmaceutical composition on the basis of the compound identified as a lead compound are described elsewhere in this specification.

Additionally, the invention relates to a method for identifying a compound that specifically binds to a polypeptide having an amino acid sequence selected from the group consisting of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; comprising the steps of (1) providing said polypeptide; and (2) identifying a compound that is capable of binding said polypeptide.

Based on the function of these proteins in DCM development a cell based assay can be developed to identify potential inhibitors or activators. The protein under investigation is expressed in cardiomyocytes (e.g. by infection with recombinant adenovirus). The expression of these proteins lead to characteristic morphological alterations. Reversal or reduction of these morphological alterations can be used in a HTS assay to identify compounds which act as inhibitors or activators of these proteins. The system can be automated by use of digital image analysis systems.

Another possibility is to identify first proteins which are binding partners of the claimed proteins. This is especially important for structural proteins or adaptor proteins in signal transduction pathways.

Methods to identify compounds capable of binding are affinity chromatography with immobilised target protein and subsequent elution of bound proteins (e.g. by acid pH), co-immmunoprecipitation and as a third method chemical crosslinking with subsequent analysis on SDS-PAGE.

The influence of compounds on these protein-protein interactions can be monitored by techniques like optical spectroscopy (e.g. fluorescence or surface plasmon resonance), calorimetry (isothermal titration microcalorimetry) and NMR. In the case of optical spectrosopy either the intrinsic protein fluorescence may change (in intensity and/or wavelength of emission maximum) upon complex formation with the binding compound or the fluorescence of a covalently attached fluorophore may change upon complex formation. The claimed protein or its identified binding partner may be labelled on e.g. cysteine or lysine residues with a fluorophore (for a collection of fluorophores see catalogues of Molecular Probes or Pierce Chemical Company) which changes its optical properties upon binding. These changes in the intrinsic or extrinsic fluorescence may be applied for use in a HTS assay to identify compounds capable of inhibiting or activating the mentioned protein-protein interaction.

If the claimed protein exhibits enzymatic activity (e.g. Kinase, Protease, Phosphatase) the inhibition or activation of this activity may be monitored by using labelled (fluorescently, radioactively or immunologically) derivates of the substrate. This activity assay which is based on labelled substrates can be used for development of a HTS assay to identify compounds acting as inhibitors or activators.

Further the invention relates to a monoclonal antibody or derivative thereof that specifically binds to polypeptide having an amino acid sequence selected from the group consisting of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676].

Moreover, the invention relates to a method for identifying a compound that increases or decreases the level in heart tissue of an mRNA encoding a polypeptide selected from the group consisting of: (a) the polypeptide having amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, the method comprising the steps of (1) contacting a DNA giving rise to said mRNA under conditions that would permit transcription of said mRNA with a test compound; and (2) detecting an increased or decreased level of the mRNA relative to the level of transcription obtained in the absence of the test compound.

This embodiment of the invention is very similar to the previously discussed one with the exception that here mRNA levels are detected whereas in the previous embodiment protein levels are detected. Methods of assessing RNA levels which also apply to this embodiment have been described herein above.

Furthermore, the invention relates to a transgenic non-human mammal whose somatic and germ cells comprise at least one gene encoding a functional or disrupted polypeptide selected from the group consisting of: (a) the polypeptide having the amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, that has been modified, said modification being sufficient to decrease or increase the amount of said functional polypeptide expressed in the heart tissue of said transgenic non-human mammal, wherein said transgenic non-human mammal exhibits a disease of the heart.

A method for the production of a transgenic non-human animal, for example transgenic mouse, comprises introduction of the aforementioned polynucleotide or targeting vector into a germ cell, an embryonic cell, stem cell or an egg or a cell derived therefrom. The non-human animal can be used in accordance with a screening method of the invention described herein. Production of transgenic embryos and screening of those can be performed, e.g., as described by A. L. Joyner Ed., Gene Targeting, A Practical Approach (1993), Oxford University Press. The DNA of the embryonal membranes of embryos can be analyzed using, e.g., Southern blots with an appropriate probe; see supra. A general method for making transgenic non-human animals is described in the art, see for example WO 94/24274. For making transgenic non-human organisms (which include homologously targeted non-human animals), embryonal stem cells (ES cells) are preferred. Murine ES cells, such as AB-1 line grown on mitotically inactive SNL76/7 cell feeder layers (McMahon and Bradley, Cell 62:1073-1085 (1990)) essentially as described (Robertson, E. J. (1987) in Teratocarcinomas and Embryonic Stem Cells: A Practical Approach. E. J. Robertson, ed. (Oxford: IRL Press), p. 71-112) may be used for homologous gene targeting. Other suitable ES lines include, but are not limited to, the E14 line (Hooper et al., Nature 326:292-295 (1987)), the D3 line (Doetschman et al., J. Embryol. Exp. Morph. 87:27-45 (1985)), the CCE line (Robertson et al., Nature 323:445-448 (1986)), the AK-7 line (Zhuang et al., Cell 77:875-884 (1994)). The success of generating a mouse line from ES cells bearing a specific targeted mutation depends on the pluripotence of the ES cells (i.e., their ability, once injected into a host developing embryo, such as a blastocyst or morula, to participate in embryogenesis and contribute to the germ cells of the resulting animal). The blastocysts containing the injected ES cells are allowed to develop in the uteri of pseudopregnant nonhuman females and are born as chimeric mice. The resultant transgenic mice are chimeric for cells having either the recombinase or reporter loci and are backcrossed and screened for the presence of the correctly targeted transgene (s) by PCR or Southern blot analysis on tail biopsy DNA of offspring so as to identify transgenic mice heterozygous for either the recombinase or reporter locus/loci.

The transgenic non-human animals may, for example, be transgenic mice, rats, hamsters, dogs, monkeys, rabbits, pigs, or cows. Preferably, said transgenic non-human animal is a mouse.

In a preferred embodiment of the transgenic non-human mammal of the invention said functional or disrupted gene was introduced into the non-human mammal or an ancestor thereof, at an embryonic stage.

In a further preferred embodiment of the transgenic non-human mammal of the invention the modification is inactivation, suppression or activation of said gene(s) or leads to the reduction or enhancement of the synthesis of the corresponding protein(s).

This embodiment allows for example the study of the interaction of various mutant forms of the aforementioned polypeptides on the onset of the clinical symptoms of a disease related to disorders in the heart. All the applications that have been herein before discussed with regard to a transgenic animal also apply to animals carrying two, three or more transgenes for example encoding different aforementioned nucleic acid molecules. It might be also desirable to inactivate protein expression or function at a certain stage of development and/or life of the transgenic animal. This can be achieved by using, for example, tissue specific, developmental and/or cell regulated and/or inducible promoters which drive the expression of, e.g., an antisense or ribozyme directed against the RNA transcript encoding the corresponding RNA; see also supra. A suitable inducible system is for example tetracycline-regulated gene expression as described, e.g., by Gossen and Bujard (Proc. Natl. Acad. Sci. 89 USA (1992), 5547-5551) and Gossen et al. (Trends Biotech. 12 (1994), 58-62). Similar, the expression of the mutant protein(s) may be controlled by such regulatory elements.

As mentioned, the invention also relates to a transgenic non-human animal, preferably mammal and cells of such animals which cells contain (preferably stably integrated into their genome) at least one of the aforementioned nucleic acid molecule(s) or part thereof, wherein the transcription and/or expression of the nucleic acid molecule or part thereof leads to reduction of the synthesis of (a) corresponding protein(s). In a preferred embodiment, the reduction is achieved by an anti-sense, sense, ribozyme, co-suppression and/or dominant mutant effect. “Antisense” and “antisense nucleotides” means DNA or RNA constructs which block the expression of the naturally occurring gene product.

Techniques how to achieve this are well known to the person skilled in the art. These include, for example, the expression of antisense-RNA, ribozymes, of molecules which combine antisense and ribozyme functions and/or of molecules which provide for a co-suppression effect; see also supra. When using the antisense approach for reduction of the amount of said proteins in cells, the nucleic acid molecule encoding the antisense-RNA is preferably of homologous origin with respect to the animal species used for transformation. However, it is also possible to use nucleic acid molecules which display a high degree of homology to endogenously occurring nucleic acid molecules encoding such a protein. In this case the homology is preferably higher than 60%, preferably higher than 80%, particularly higher than 90%, more preferably higher than 95% and especially higher than 99%.

In cases where more than one of the aforementioned genes are inactivated, interrelationships of gene products in the onset or progression of the diseases of the heart may be assessed. In this regard, it is also of interest to cross transgenic non-human animals having different transgenes for assessing further interrelationships of gene products in the onset or progression of said disease. Consequently, the offspring of such crosses is also comprised by the scope of the present invention.

In addition, the invention relates to a method for identifying in heart issue a compound that increases or decreases the expression of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, said method comprising the steps of: (1) contacting a transgenic non-human mammal as described herein above with a test compound, and (2) detecting an increased/decreased level of expression of said polypeptide relative to the expression in the absence of said test compound.

The test compound which has preferably been tested beforehand for essentially lacking toxicity for the animal can be administered to the animal by any convenient route suitable for administration. These routes include injection, topical and oral administration. Intervals and doses of administration may vary and will be decided upon by the physician/researcher on a case-by-case basis.

Detection, if any, may be effected by a variety of means. For example, if the transgene includes a bioluminescent portion, increase of polypeptide production may be assessed as described, for example, in EP 95 94 1424.4 or in EP 99 12 4640.6. Alternatively, and if the polypeptides are present in the bloodstream, blood of the non-human transgenic animal may be assessed for the changing quantity of the protein. It is preferred in such a case that the gene encoding the polypeptide of interest carries an inducible promoter. Thus, by comparing the situations with and without induction, it can conveniently be determined whether the test compound has indeed an effect on the polypeptide produced or whether the test compound causes an effect unrelated to the level of polypeptide produced. In certain embodiments of the invention, the non-human transgenic animal will have to be sacrificed in order to assess whether a change in the level of polypeptide expression has occurred. For example, heart tissue may be removed from the sacrificed animal and assessed, using standard technologies, for the expression level of the protein. For example, an antibody specific for the polypeptide may be contacted with the heart tissue and the test developed with a second labeled antibody that is directed to the first antibody. Alternatively, the first antibody itself may be labeled. Heart tissue of a non-human transgenic animal that has been contacted with the test compound would be compared with heart tissue of a non-human transgenic animal that has not been contacted with said test compound.

As mentioned herein above, the transgenic animal may carry more than one of the aforementioned nucleic acid molecules. Accordingly, the effect of a test compound on the expression level of any of these transgenes may be assessed. In addition, a variety of test compounds may be tested, at the same time, for the effect on one or a variety of said transgenes.

A test compound that has proven to be effective in increasing or decreasing the level of the polypeptide of interest and/or in decreasing or increasing the turnover of the polypeptide of interest may be either directly formulated into a medicament (if, for example, its structure is suitable for administration and if it has proven to be non-toxic) or may serve as a lead compound for downstream developments, the results of which may then be formulated into pharmaceutical compositions.

In a preferred embodiment of the method of the invention the test compound prevents or ameliorates a disease of the heart in said transgenic non-human mammal.

In this embodiment, the effect of the test compound may be assessed by observing the disease state of the transgenic animal. Thus, if the animal suffers from a disease of the heart prior to the administration of the test compound and the administration of the test compound results in an amelioration of the disease, then it can be concluded that this test compound is a prime candidate for the development of a medicament useful also in humans. In addition the compound could also inhibit disease establishment by treatment in advance.

A further embodiment of the invention is a method for identifying one or a plurality of isogenes of a gene coding for a polypeptide selected from the group consisting of: the amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; the method comprising the steps of

(1) providing nucleic acid coding for said polypeptide or a part thereof; and

(2) identifying a second nucleic acid that (i) has a homology of 60%, preferably 80%, especially 90%, advantageously 99% or (ii) hybridizes in 4×SSC, 0.1 SDS at 45° C. to the nucleic acid molecule encoding the amino acid sequence of (a), (c) or (d).

The term isogenes shall mean genes that are thought to be created by gene duplication. They can be identified by comparing the homology of the DNA-, RNA-, or protein-sequence of interest with other DNA, RNA or protein-sequences of the same species from different databases. There might be strong differences in the degree of homology between isogenes of the same species. This may be dependent on the time-point, when the gene duplication event took place in evolution and the degree of conservation during evolution.

Isogenes can be identified and cloned by RT-PCR as has been demonstrated by Screaton et al. (1995) EMBO J. 14:4336-4349 or Huang et al. (1998) Gene 211: 49-55. Isogenes can also be identified and cloned by colony hybridisation or plaque hybridization as described in Sambrook, Fritsch, Maniatis (1989), Molecular Cloning. Cold Spring Harbor Laboratory Press. In a first step, either a genomic or a cDNA library in bacteria or phages is generated. In order to identify isogenes, colony hybridisation or plaque hybridization is slightly modified in a way that cross-hybridizations are detectable under conditions of lower stringency. This can be achieved by lowering the calculated temperature for hybridisation and washing and/or by lowering the salt concentration of the washing solutions (Sambrook, Fritsch, Maniatis (1989) Cold Spring Harbor Laboratory Press). For example, a low-stringency washing condition may include 2 wash steps at a temperature between 45° C. and 65° C. with 4×SSC, 0.1% SDS for 30 min (50 ml) and finally two wash steps with 50 ml of a solution containing 2×SSC, 0.1% SDS for 30 min. After detection, signal intensity of colonies containing an isogene is dependent on the homology of a gene and its isogene(s).

Furthermore, the invention relates to a method for identifying one or a plurality of genes whose expression in heart tissue is modulated by inhibiting, decreasing or increasing the expression of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, or of an mRNA encoding said polypeptide, said modulation being indicative of a disease of the heart, said method comprising the steps of: (1) contacting a plurality of heart tissue cells with a compound that inhibits, decreases or increases the expression of said polypeptide under conditions that permit the expression of said polypeptide in the absence of a test compound, and (2) comparing a gene expression profile of said heart cell in the presence and in the absence of said compound.

The term “gene expression profile” shall mean all expressed genes of a cell or a tissue. Such profile can be assessed using the methods well known in the art, for example isolation of total RNA, isolation of poly(A) RNA from total RNA, suppression subtractive hybridization, differential display, preparation of cDNA libraries or quantitative dot blot analysis, as for example described in Example 1 of this application.

This embodiment of the method of the invention is particularly suitable for identifying further genes the expression level of which is directly affected by the aberrant expression of any of the aforementioned genes. In other words, this embodiment of the method of the invention allows the identification of genes involved in the same protein cascade as the aberrantly expressed gene. Typically, the method of the invention will be a method performed in cell culture.

The method of the invention allows for the design of further medicaments that use other targets than the aberrantly expressed gene. For example, if a potential target downstream of the aberrantly expressed gene is indeed targeted by a medicament, the negative effect of the aberrantly expressed gene may be efficiently counterbalanced. Compounds modulating other genes in the cascade may have to be refined or further developed prior to administration as a medicament as described elsewhere in this specification.

Additionally, the invention relates to a method for identifying one or a plurality of genes whose expression in heart tissue is modulated by the inhibition, decreasing or increasing of the expression of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, or of an mRNA encoding said polypeptide, said modulation being indicative of a disease of the heart, said method comprising the steps of: (1) providing expression profiles of (i) a plurality of heart tissue cells from or derived from a heart of a subject suffering from a disease of the heart; and (ii) a plurality of heart tissue cells from or derived from a subject not suffering from a disease of the heart; and (2) comparing the expression profiles (i) and (ii).

In variation to the method described herein above, this embodiment of the method of the invention compares the expression profiles of cells from a healthy subject and a subject suffering from a heart disease. In this regard, the term “cells derived from a heart” includes cells that are held in cell culture or even cell lines that autonomously grow in cell culture and that were originally derived from heart tissue. By comparing the two expression profiles, differences in expression levels of genes involved in the disease of the heart may be identified. As with the preceding embodiment, these genes may be part of a cascade involving the aberrantly expressed gene. Examples of such cascades are signaling cascades. Once genes are identified that are expressed at a different level in a diseased heart, they may be tested up-regulation or down-regulation by bringing them into contact with suitable test compounds. Again, these test compounds may then, with or without further development, be formulated into pharmaceutical compositions.

In a preferred embodiment, the method of the invention further comprises the steps of (3) determining at least one gene that is expressed at a lower or higher level in the presence of said compound; and (4) identifying a further compound that is capable of raising or lowering the expression level of said at least one gene.

This preferred embodiment of the invention requires that one of the genes the expression of which may directly or indirectly be lowered or increased by the expression of the aberrant gene is identified. Then, a further panel of test compounds may be tested for the capacity to increase or decrease the expression of said further gene. Compounds that are successfully tested would be prime candidates for the development of medicaments for the prevention or treatment of a disease of the heart.

In another preferred embodiment, the method of the invention further comprises the steps of (3) determining at least one gene that is expressed at a lower or higher level in said heart tissue cells from or derived from a heart of a subject suffering from a disease of the heart; and (4) identifying a further compound that is capable of raising or lowering the expression level of said at least one gene.

In variation of the previously discussed embodiment, this embodiment requires that at least one gene is identified by comparing the expression profiles of tissue or cells derived from a healthy subject and from a subject suffering from a disease of the heart. Subsequently, at least one compound is identified that is capable of increasing or decreasing the expression of said gene.

In yet another preferred embodiment, the method of the invention further comprises the steps of (3) determining at least one gene that is expressed at a higher or lower level in the presence of said compound; and (4) identifying a further compound that is capable of reducing or raising the expression level of said at least one gene.

In this and the following embodiment, the situation is covered that another gene in the cascade that also includes the aberrantly expressed gene has a higher or lower expression level that needs to be lowered or raised in order to effectively treat the disease of the heart. Again, once such a gene is identified, a compound is tested for its capacity to lower expression of said gene.

In still another preferred embodiment, the method of the invention further comprises the steps of (3) determining at least one gene that is expressed at a higher or lower level in said heart tissue cells from or derived from a heart of a subject suffering from a disease of the heart; and (4) identifying a further compound that is capable of reducing or enhancing the expression level of said at least one gene.

Additionally, the invention relates to a method for identifying proteins or a plurality of proteins whose activity is modulated by a polypeptide having the amino acid sequence selected from the group consisting of SEQ ID NO: 1 [NP 003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; the method comprising the steps of (1) providing said polypeptide and (2) identifying a further protein that is capable of interacting with said polypeptide.

One possible method to identify protein-protein interactions is the Yeast two-hybrid screen described by Golemis & Khazak (1997), Methods Mol Biol. 63:197-218. Other well established methods in order to identify protein-protein interactions are co-immunoprecipitations or in vitro protein interaction assays like GST-pulldown assays (such as described in Stoss, Schwaiger, Cooper and Stamm (1999). J. Biol. Chem. 274: 10951-10962).

In a further preferred embodiment of the method of the invention said compound is a small molecule or a peptide derived from an at least partially randomized peptide library.

Additionally, the invention relates to a method of refining a compound identified by the method as described herein above comprising the steps of (1) identification of the binding sites of the compound and the DNA or mRNA molecule by site-directed mutagenesis or chimeric protein studies; (2) identification of the binding-site of said polypeptide and the compound by site-directed mutagenesis of the corresponding DNA or by chimeric protein studies, (3) molecular modeling of both the binding site of the compound and the binding site of the DNA or mRNA molecule; and (4) modification of the compound to improve its binding specificity for the DNA or mRNA.

All techniques employed in the various steps of the method of the invention are conventional or can be derived by the person skilled in the art from conventional techniques without further ado. Thus, biological assays based on the herein identified nature of the polypeptides may be employed to assess the specificity or potency of the drugs wherein the increase of one or more activities of the polypeptides may be used to monitor said specificity or potency. Steps (1) and (2) can be carried out according to conventional protocols. A protocol for site directed mutagenesis is described in Ling M M, Robinson B H. (1997) Anal. Biochem. 254: 157-178. The use of homology modelling in conjunction with site-directed mutagenesis for analysis of structure-function relationships is reviewed in Szklarz and Halpert (1997) Life Sci. 61:2507-2520. Chimeric proteins are generated by ligation of the corresponding DNA fragments via a unique restriction site using the conventional cloning techniques described in Sambrook, Fritsch, Maniatis. Molecular Cloning, a laboratory manual. (1989) Cold Spring Harbor Laboratory Press. A fusion of two DNA fragments that results in a chimeric DNA fragment encoding a chimeric protein can also be generated using the gateway-system (Life technologies), a system that is based on DNA fusion by recombination. A prominent example of molecular modelling is the structure-based design of compounds binding to HIV reverse transcriptase that is reviewed in Mao, Sudbeck, Venkatachalam and Uckun (2000). Biochem. Pharmacol. 60: 1251-1265.

For example, identification of the binding site of said drug by site-directed mutagenesis and chimerical protein studies can be achieved by modifications in the (poly)peptide primary sequence that affect the drug affinity, this usually allows to precisely map the binding pocket for the drug.

As regards step (2), the following protocols may be envisaged: Once the effector site for drugs has been mapped, the precise residues interacting with different parts of the drug can be identified by combination of the information obtained from mutagenesis studies (step (1)) and computer simulations of the structure of the binding site provided that the precise three-dimensional structure of the drug is known (if not, it can be predicted by computational simulation). If said drug is itself a peptide, it can be also mutated to determine which residues interact with other residues in the polypeptide of interest.

Finally, in step (3) the drug can be modified to improve its binding affinity or ist potency and specificity. If, for instance, there are electrostatic interactions between a particular residue of the polypeptide of interest and some region of the drug molecule, the overall charge in that region can be modified to increase that particular interaction.

Identification of binding sites may be assisted by computer programs. Thus, appropriate computer programs can be used for the identification of interactive sites of a putative inhibitor and the polypeptide by computer assisted searches for complementary structural motifs (Fassina, Immunomethods 5 (1994), 114-120). Further appropriate computer systems for the computer aided design of protein and peptides are described in the prior art, for example, in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. Modifications of the drug can be produced, for example, by peptidomimetics and other inhibitors can also be identified by the synthesis of peptidomimetic combinatorial libraries through successive chemical modification and testing the resulting compounds. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, the three-dimensional and/or crystallographic structure of activators of the expression of the polypeptide of the invention can be used for the design of peptidomimetic activators, e.g., in combination with the (poly)peptide of the invention (Rose, Biochemistry 35 (1996), 12933-12944; Rutenber, Bioorg. Med. Chem. 4 (1996), 1545-1558).

In accordance with the above, in a preferred embodiment of the method of the invention said compound is further refined by peptidomimetics.

The invention furthermore relates to a method of modifying a compound identified or refined by the method as described herein above as a lead compound to achieve (1) modified site of action, spectrum of activity, organ specificity, and/or (2) improved potency, and/or (3) decreased toxicity (improved therapeutic index), and/or (4) decreased side effects, and/or (5) modified onset of therapeutic action, duration of effect, and/or (6) modified pharmakinetic parameters (resorption, distribution, metabolism and excretion), and/or (7) modified physico-chemical parameters (solubility, hygroscopicity, color, taste, odor, stability, state), and/or (8) improved general specificity, organ/tissue specificity, and/or (9) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophylic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetates, or (xiv) N-acetylation to amides, phenylcarbamates, or (xv) synthesis of Mannich bases, imines, or (xvi) transformation of ketones or aldehydes to Schiff's bases, oximes, acetates, ketales, enolesters, oxazolidines, thiozolidines or combinations thereof.

The various steps recited above are generally known in the art. They include or rely on quantitative structure-action relationship (QSAR) analyses (Kubinyi, “Hausch-Analysis and Related Approaches”, VCH Verlag, Weinheim, 1992), combinatorial biochemistry, classical chemistry and others (see, for example, Holzgrabe and Bechtold, Deutsche Apotheker Zeitung 140(8), 813-823, 2000).

The invention additionally relates to a method for inducing a disease of the heart in a non-human mammal, comprising the step of contacting the heart tissue of said mammal with a compound that inhibits, decreases or increases the expression of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution.

This embodiment of the invention is particularly useful for mimicking factors/developments leading to the onset of the disease. The fact, that differences in the expression of a protein contributes to heart failure has been shown for phospholamban, for example. Mice over-expressing phospholamban develop heart failure. This effect is thought to be due to the inhibition of Serca. (Minamisawa et al. (1999) Cell, 99:313-322).

In a preferred embodiment of the method of the invention said compound that decreases or increases is a small molecule, an antibody or an aptamer that specifically binds said polypeptide.

The terms “small molecule” as well as “antibody” have been described herein above and bear the same meaning in connection with this embodiment.

The invention moreover relates to a method of producing a pharmaceutical composition comprising formulating the compound identified, refined or modified by the method as described herein above, optionally with a pharmaceutically active carrier and/or diluent. The pharmaceutical composition of the present invention may further comprise a pharmaceutically acceptable carrier and/or diluent. Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical, intradermal, intranasal or intrabronchial administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 106 to 1012 copies of the DNA molecule. The compositions of the invention may be administered locally or systemically. Administration will generally be parenterally, e.g., intravenously; DNA may also be administered directly to-the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents such as interleukins or interferons depending on the intended use of the pharmaceutical composition.

The invention also relates to a method for preventing or treating a disease of the heart in a subject in need of such treatment, comprising the step of increasing or decreasing the level of a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the ammo acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, in the heart tissue of the subject.

Further, the invention relates to a method of preventing or treating a disease of the heart in a subject in need of such treatment comprising the step of increasing or decreasing the level of mRNA encoding a polypeptide selected from the group consisting of: (a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; (b) a polypeptide having an amino acid sequence that is at least 60%, preferably at least 80%, especially at least 90%, advantageously at least 99% identical to the amino acid sequence of (a); and (c) a polypeptide having the amino acid sequence of (a) with at least one conservative amino acid substitution, in the heart tissue of the subject.

The invention in a preferred embodiment relates to a method wherein such increase/decrease is effected by administering the pharmaceutical composition obtained by the method as described herein above.

In a further preferred embodiment of the method of the invention such an increase/decrease is effected by introducing the DNA sequence recited herein above into the germ line or into somatic cells of a subject in need thereof.

Technologies for effecting such an introduction have been described herein above.

In a most preferred embodiment of the method of the invention, the disease of the heart to be treated is congestive heart failure, dilative cardiomyopathy, hypertrophic cardiomyopathy, ischemic cardiomyopathy, specific heart muscle disease, rhythm and conduction disorders, syncope and sudden death, coronary heart disease, systemic arterial hypertension, pulmonary hypertension and pulmonary heart disease, valvular heart disease, congenital heart disease, pericardial disease or endocarditis.

In addition, the invention relates to a method for identifying subjects at risk for heart diseases, especially congestive heart failure comprising the step of detecting an increased level of MYOM2, the LIM domain, the muscle isoform of creatine kinase, YAP65, APOBEC-2, SMPX or C-193 (CARP) in the heart tissue of a subject.

The invention additionally relates to a method for preventing or treating heart diseases, especially congestive heart failure in a subject, said method comprising the step of contacting the heart tissue of said subject with a compound that decreases or increases the expression of MYOM2, the LIM domain, the muscle isoform of creatine kinase, YAP65, APOBEC-2, SMPX or C-193 (CARP).

In addition the invention relates to a method for identifying subjects at risk for heart diseases, especially congestive heart failure comprising the step of detecting decreased creatine kinase activity in the tissue of a subject, especially in a muscle tissue or from blood or serum. One possible method to detect the activity of creatine kinase would be a conventional kinetic UV-test as described by the International Federation of Clinical Chemistry and Laboratory Medicine (IFCC), 1991.

Moreover the invention relates to a method for identifying a subject at risk for heart diseases, especially congestive heart failure, said method comprising detecting increased levels of creatine phosphate in a subject, especially in the blood or serum of a subject.

The invention as well relates to a method for preventing or treating heart diseases, especially congestive heart failure in a subject, said method comprising the step of increasing the transfer of phosphoryl groups from creatine phosphate to ADP in the tissue of a subject, especially in a muscle tissue.

In a preferred embodiment of the method of the invention the activity of creatine kinase is increased in said tissue.

The invention additionally relates to a method for identifying a compound for preventing or treating heart diseases, especially congestive heart failure, said method comprising the steps of (a) contacting creatine kinase with a substrate for creatine kinase and a test compound, and (b) determining whether the transfer of phosphoryl groups from the substrate is increased in the presence of the test compound.

The figures show:

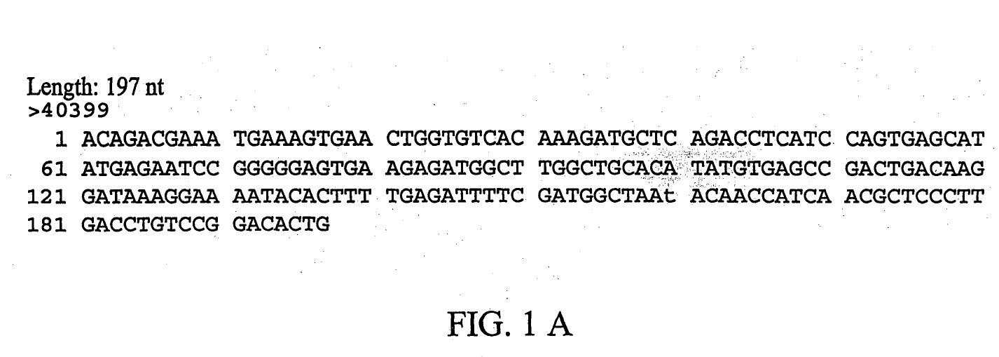

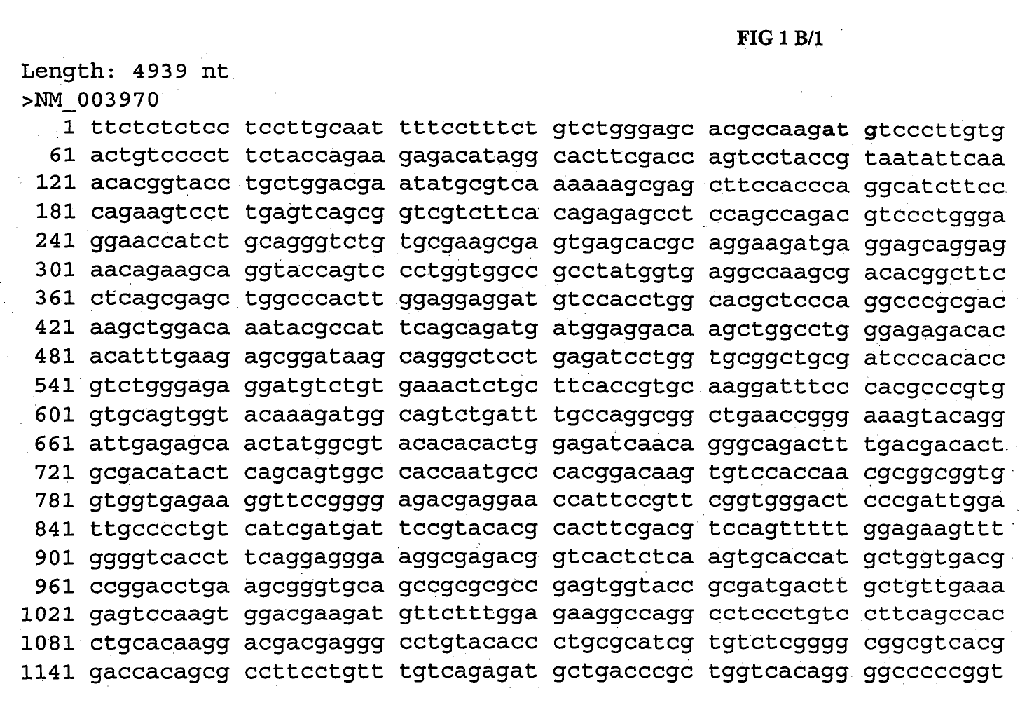

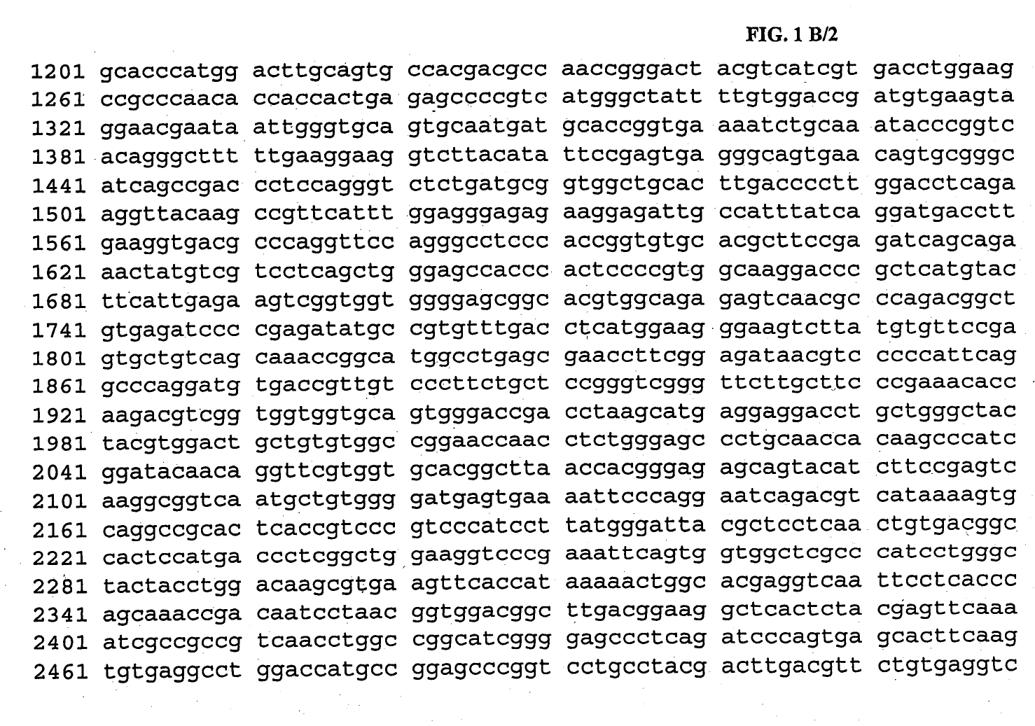

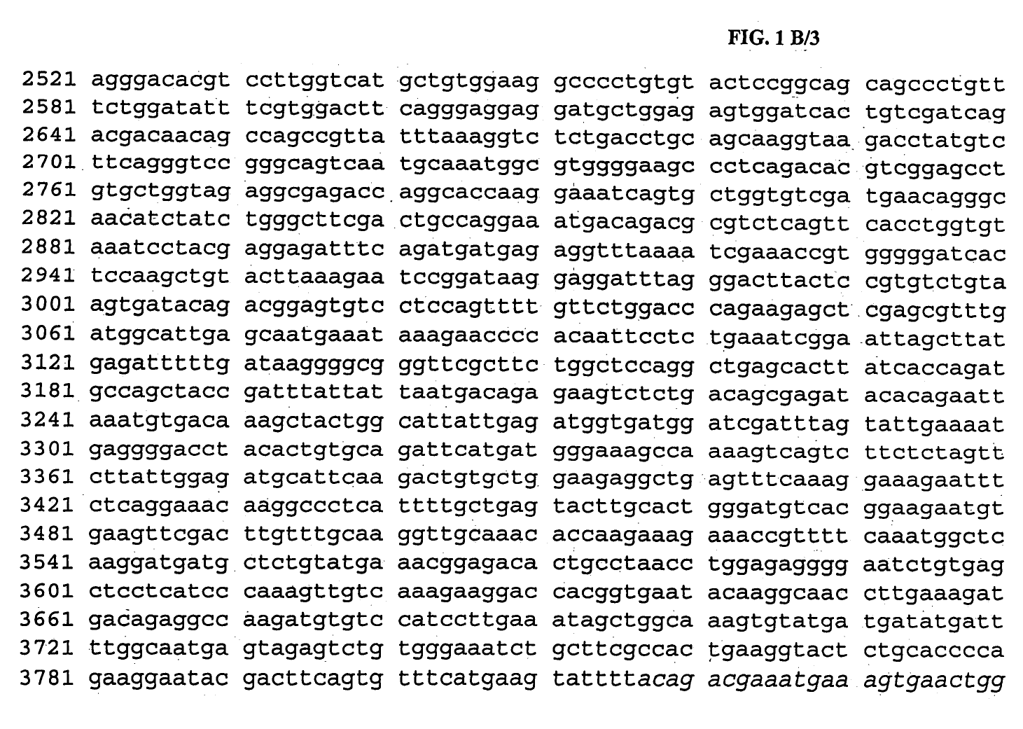

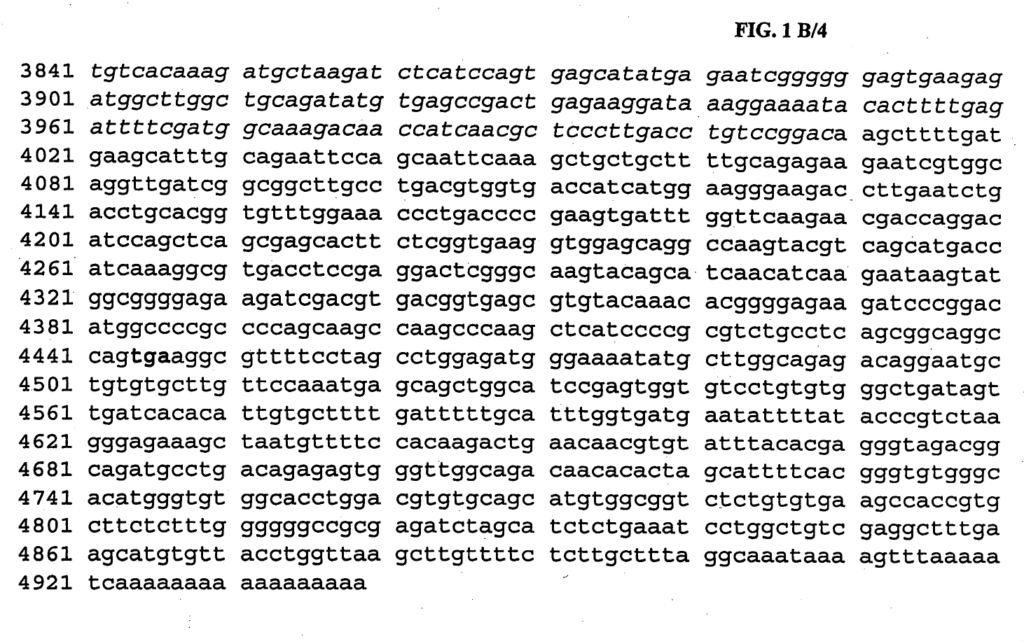

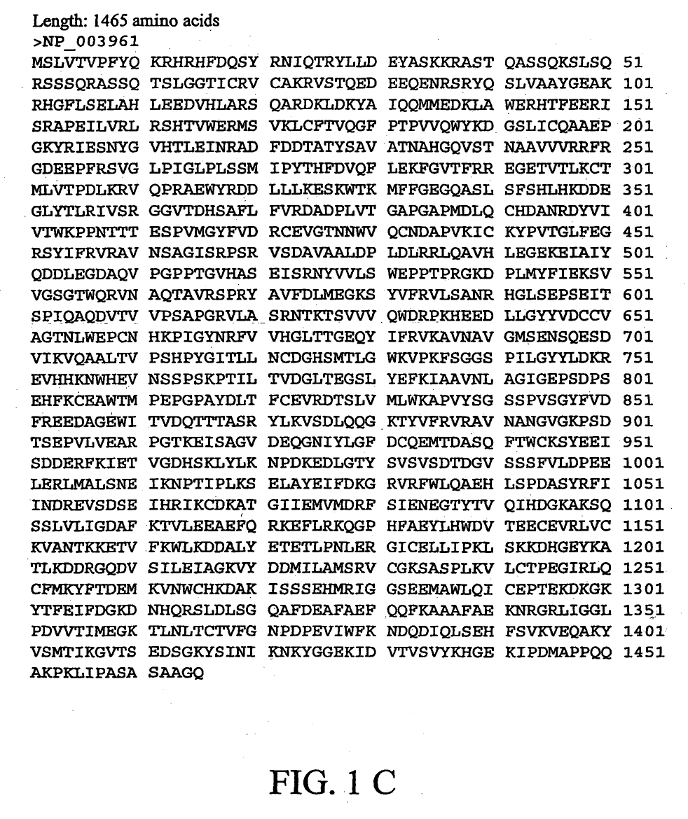

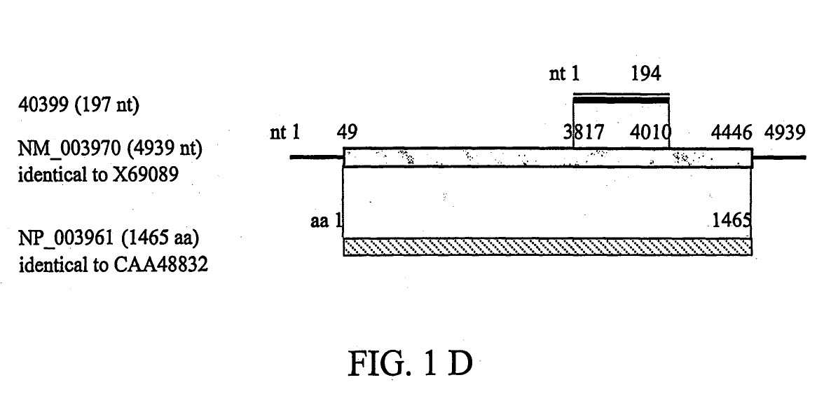

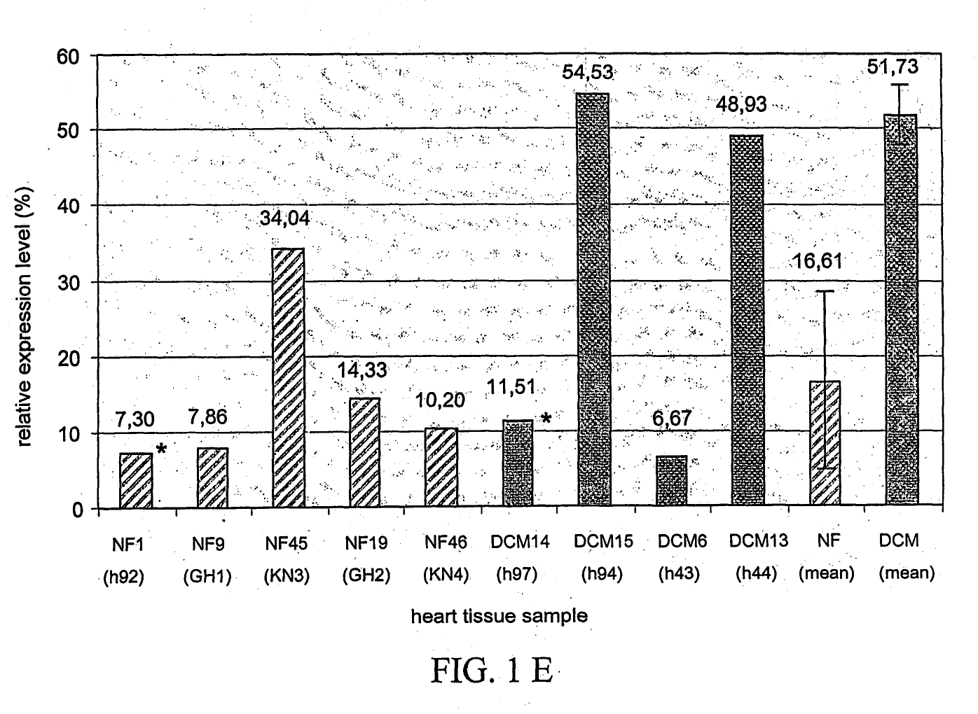

FIG. 1a shows the cDNA sequence of clone 40399 (corresponds to SEQ ID NO: 20).

FIG. 1b shows the sequence of the EST clone NM_{—}003970. Start and stop codons are marked by bold letters, the sequence of 40399 is marked in italic letters (corresponds to SEQ ID NO: 10).

FIG. 1c shows the putative amino acid sequence M-PROTEIN (MYOMESIN) 2 (MYOM2) (corresponds to SEQ ID NO: 1).

FIG. 1d shows a schematic alignment of the cDNA fragment 40399 identified in SSH with its homologous Genbank entree and the open reading frame of 1465 amino acids (aa). Not to scale. Homology scores were determined using blast2 algorithm of NCBI:

40399-NM_{—}003970: Expect=2e-88, Identities=187/194 (96%), Positives=187/194 (96%).

FIG. 1e: Two filters were hybridized sequentially with [α-33P]UTP labeled T3 transcripts from cDNA libraries prepared from mRNA of five control and four DCM heart tissues as indicated. Experiments were normalized by adjusting the overall signal intensity of each hybridization to 100%, relative expression levels are given. A mean value and standard deviation was calculated from all NF samples and DCM sample 15 and 13, respectively. Asterisks mark samples used for SSH.

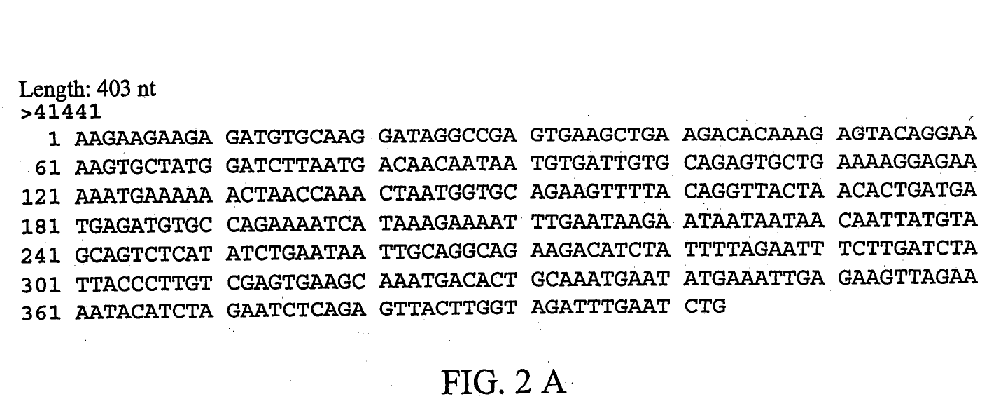

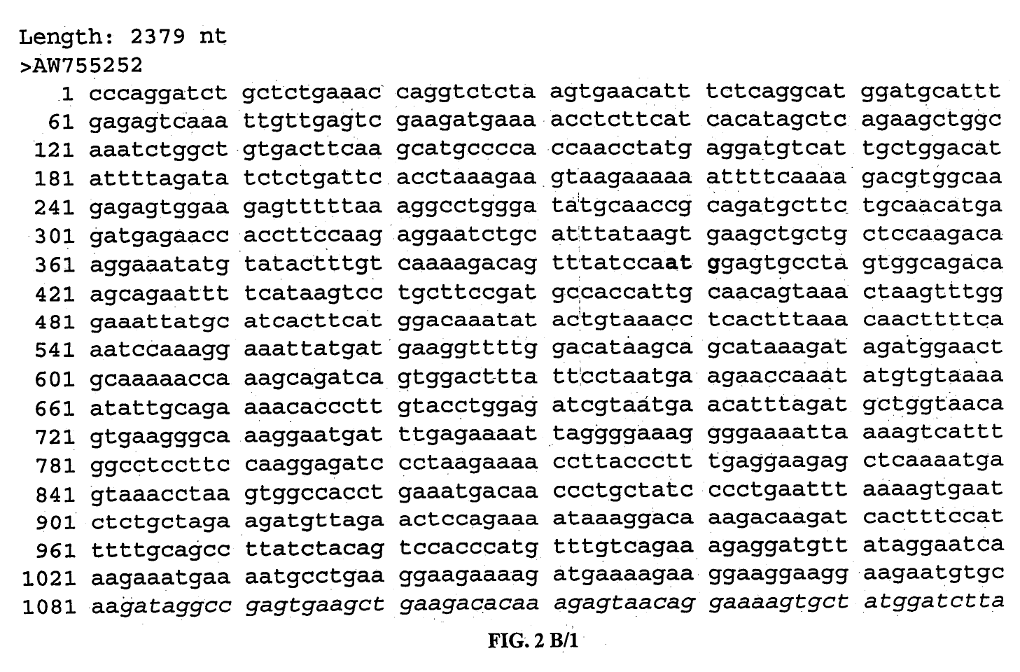

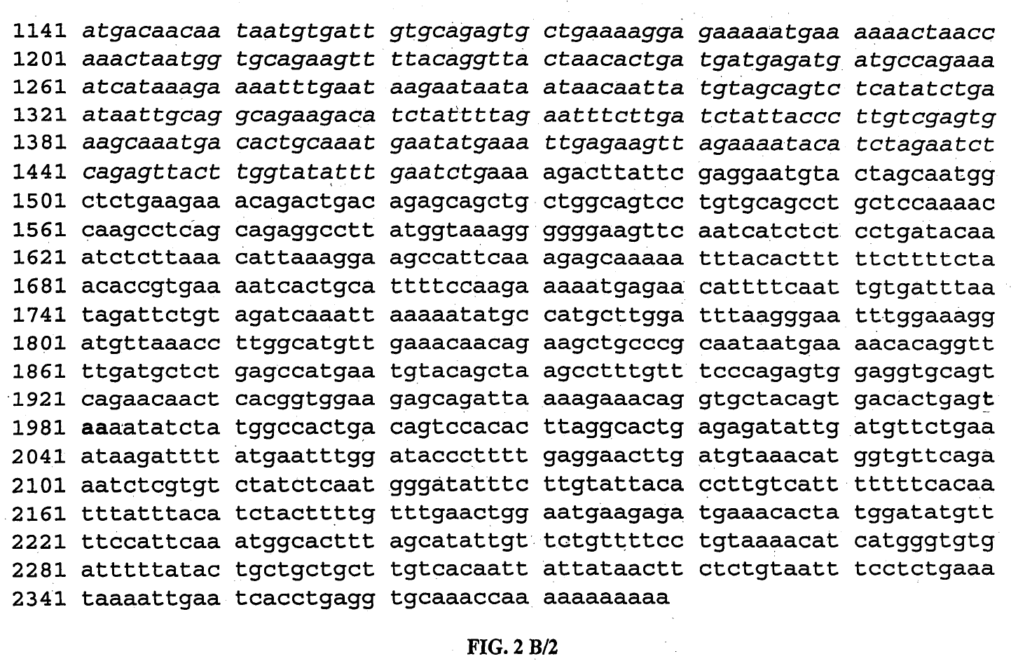

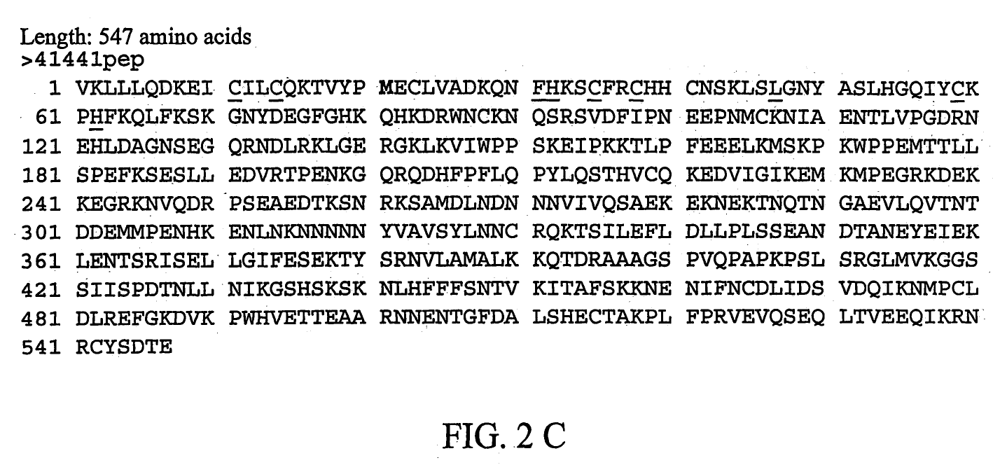

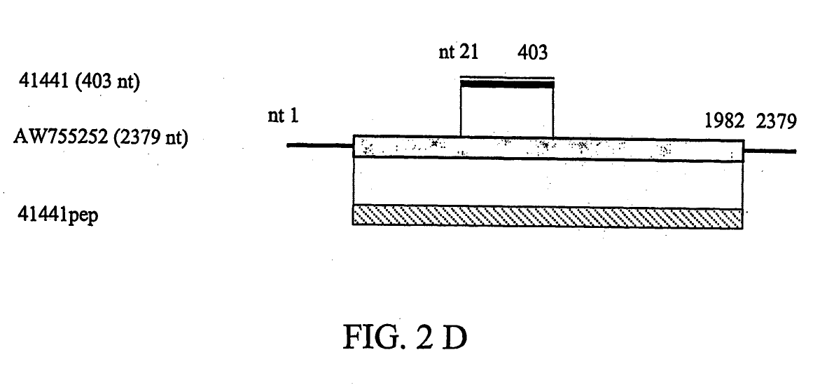

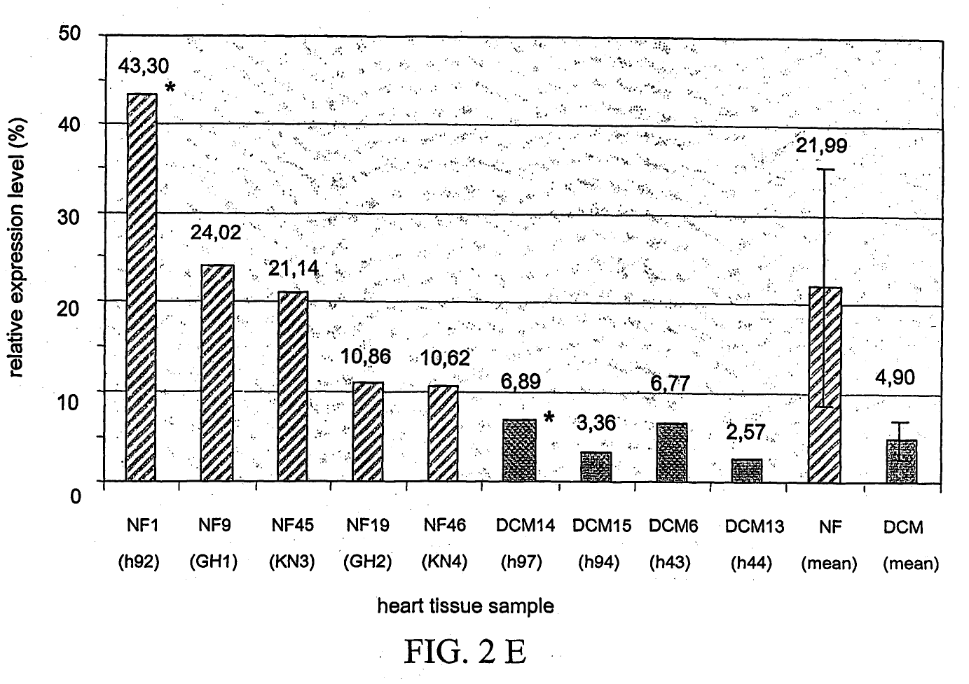

FIG. 2a shows the cDNA sequence of clone 41441 (corresponds to SEQ ID NO: 2).

FIG. 2b shows the sequence of the EST clone AW755252 (corresponds to SEQ ID NO: 11). Start and stop codons are marked in bold letters, the sequence of 41441 is given in italic letters.

FIG. 2c shows the amino acid sequence 41441pep (corresponds to SEQ ID NO: 21). The first methionine of the open reading frame is marked in bold letters. Amino acids 11-62 of 41441pep encode a cysteine-rich LIM domain (PS00478, PS50023), which is composed of 2 special zinc fingers that are joined by a 2-amino acid spacer (consensus: CX2CX15-21[FYWH]HX2[CH]X2CX2CX3[LIVMF]XnCX2H as underlined). According to this analyses, we expect the start codon to be further upstream of the first methionine in frame 1 assuming that a sequencing error exists in the 5′ region of AW755252.

FIG. 2d shows a schematic alignment of the cDNA fragment 41441 identified in SSH with its homologous Genbank entree and the predicted open reading frame. Not to scale. Homology scores were determined using blast2 algorithm of NCBI:

41441-AW755252: Expect=0.0, Identities=369/385 (95%), Positives=369/385 (95%), Gaps=2/385 (0%)

FIG. 2e: Two filters were hybridized sequentially with [α-33P]UTP labeled T3 transcripts from cDNA libraries prepared from mRNA of five control and four DCM heart tissues as indicated. Experiments were normalized by adjusting the overall signal intensity of each hybridization to 100%, relative expression levels are given. Mean values and standard deviations were calculated from all NF and DCM samples, respectively. Asterisks mark samples used for SSH.

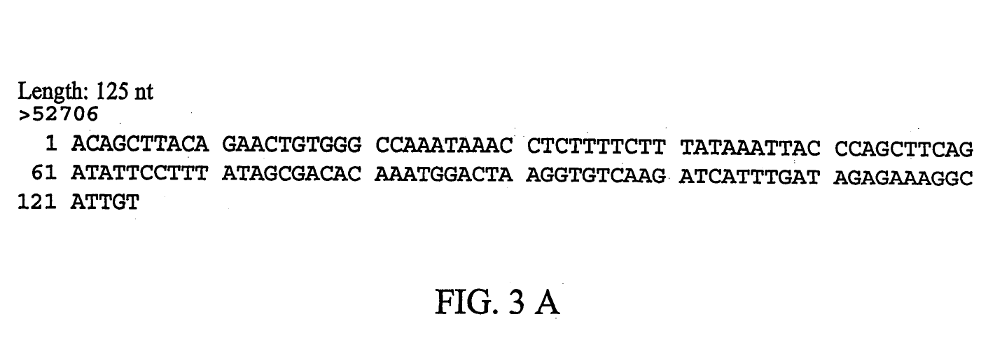

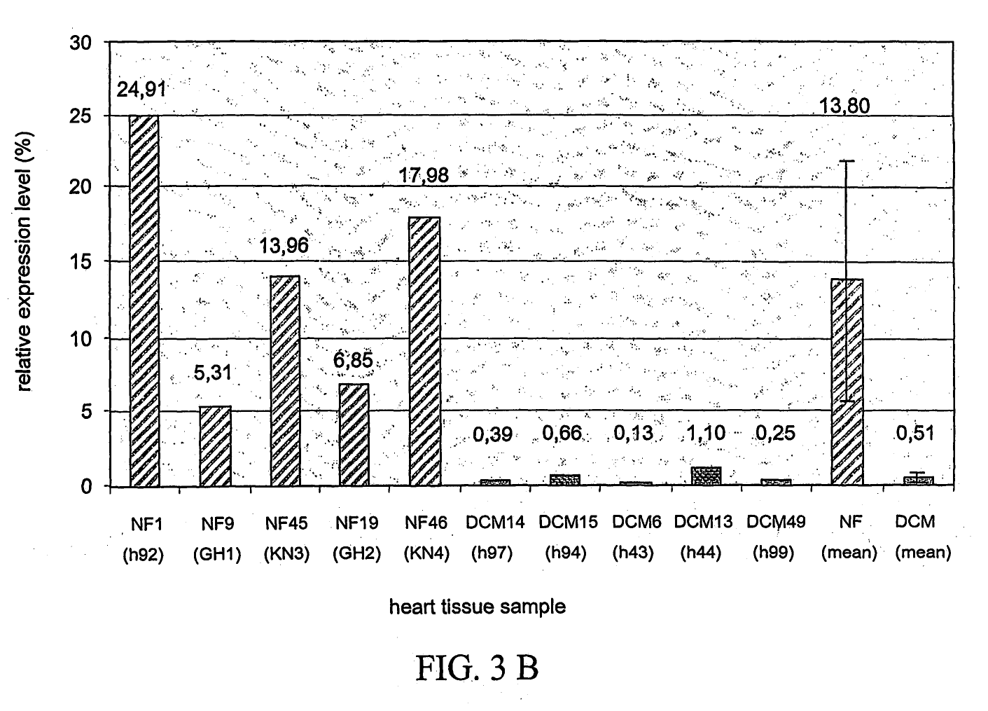

FIG. 3a shows the cDNA sequence of clone 52706 (corresponds to SEQ ID NO: 12).

FIG. 3b: Two filters were hybridized sequentially with [α-33P]UTP labeled T3 transcripts from cDNA libraries prepared from mRNA of five control, and five DCM heart tissues as indicated. Experiments were normalized by adjusting the overall signal intensity of each hybridization to 100%, relative expression levels are given.

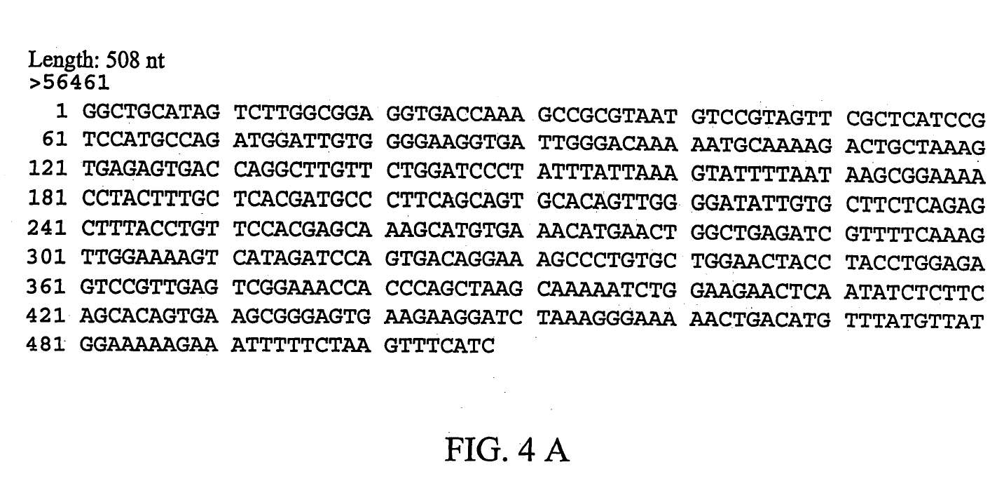

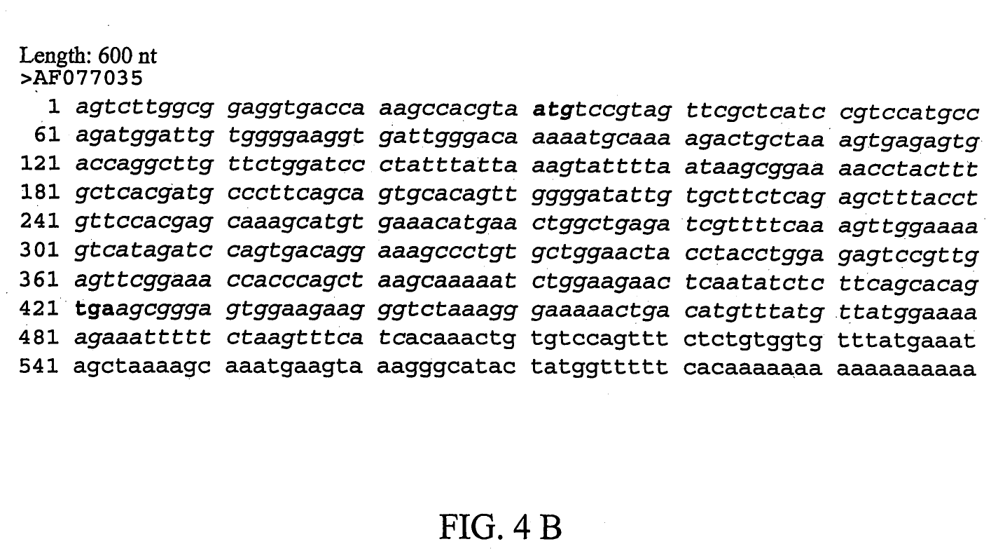

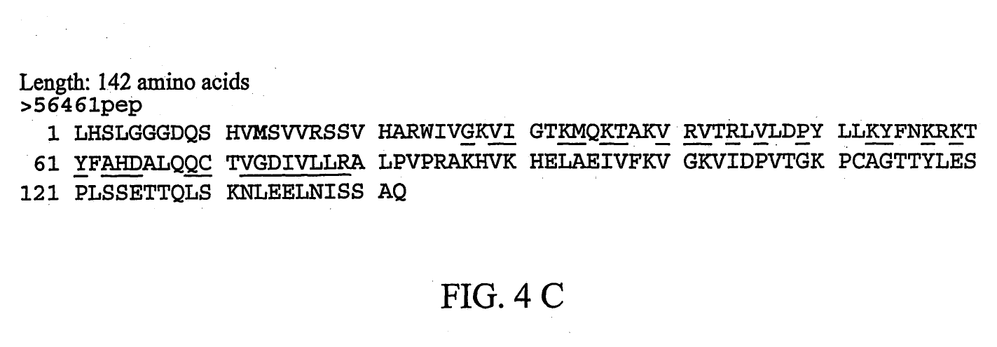

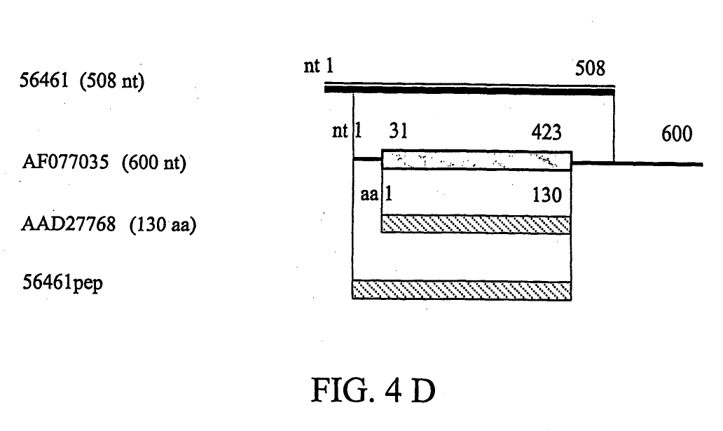

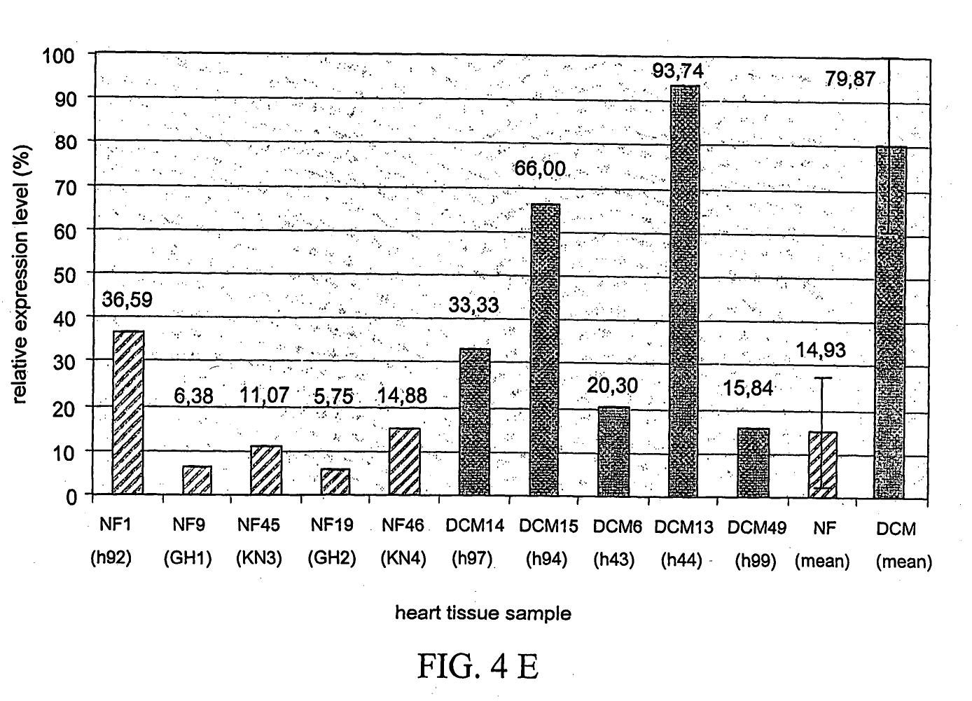

FIG. 4a shows the cDNA sequence of clone 56461 (corresponds to SEQ ID NO: 13).

FIG. 4b shows the sequence of the EST clone AF077035 (corresponds to SEQ ID NO: 22). Start and stop codons are marked in bold letters, the sequence of 56461 is marked in italic letters.

FIG. 4c shows the putative amino acid sequence AAD27768 (corresponds to SEQ ID NO: 3). The first methionine of the open reading frame is marked in bold letters. Amino acids 27-79 of 56461 are highly homologous to the rRNA binding motif of 30S ribosomal protein S 17 and 40S ribosomal protein S11 (PD001295). A cleavage site for mitochondrial presequences may be predicted for amino acids 57-61 KRK|TY (R2-motif).

FIG. 4d shows a schematic alignment of the cDNA fragment 56461 identified in SSH with its homologous Genbank entree and the open reading frame of 130 amino acids (aa). Not to scale. Homology scores were determined using blast2 algorithm of NCBI:

56461-AF077035: Expect=0.0, Identities=498/502 (99%), Positives=498/502 (99%), Gaps=2/502 (0%).

FIG. 4e: Two filters were hybridized sequentially with [α-33P]UTP labeled T3 transcripts from cDNA libraries prepared from mRNA of five control and five DCM heart tissues as indicated. Experiments were normalized by adjusting the overall signal intensity of each hybridization to 100%, relative expression levels are given. A mean value and standard deviation was calculated from all NF samples and DCM15 and DCM13, respectively.

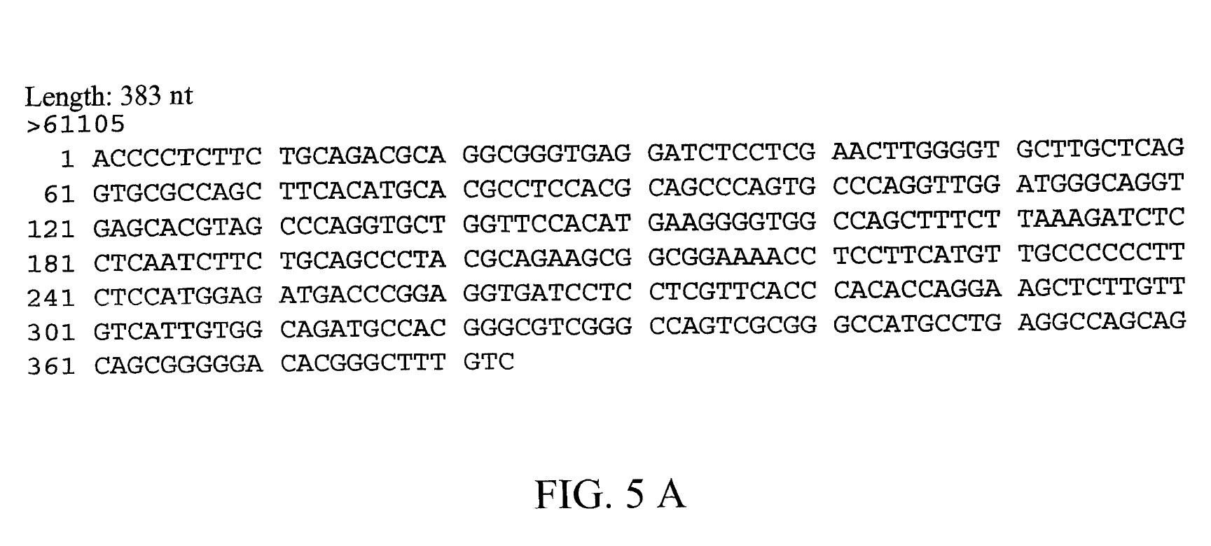

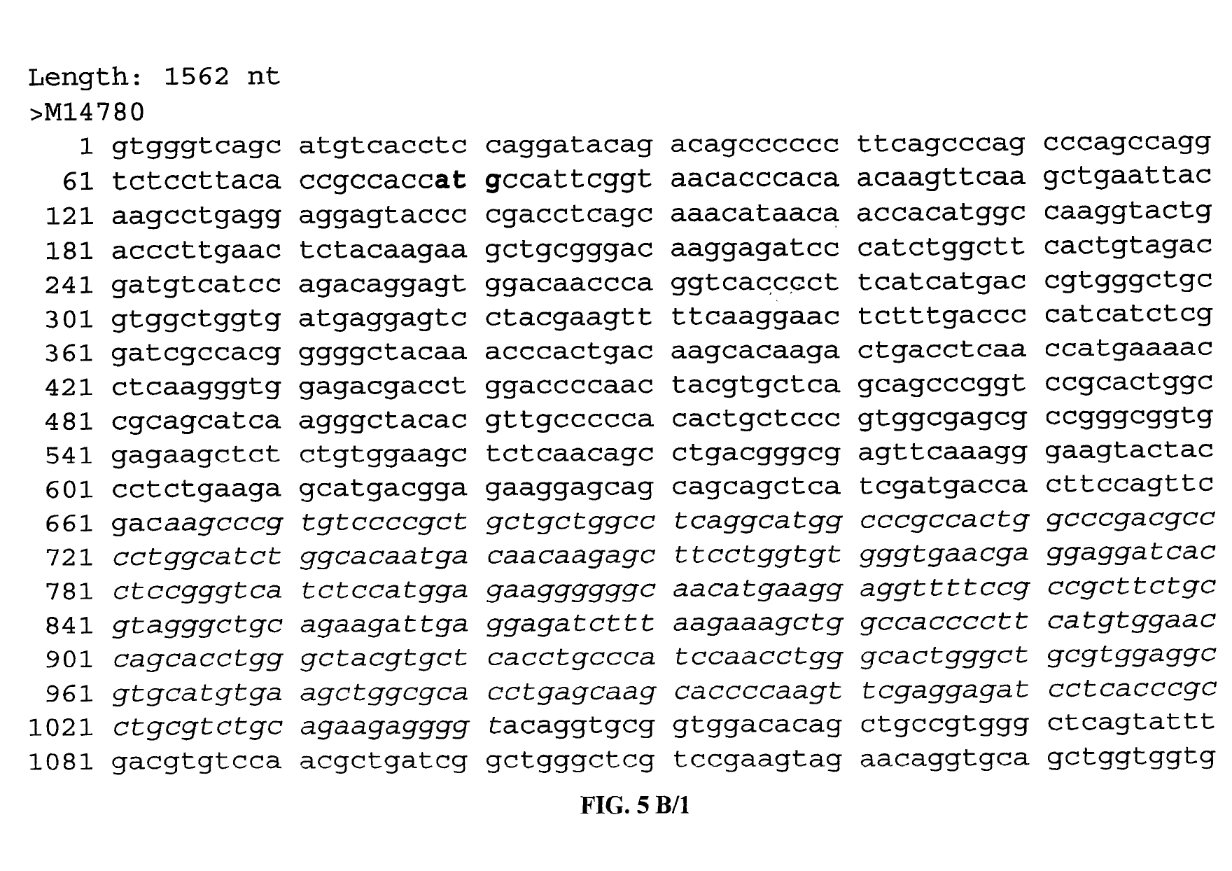

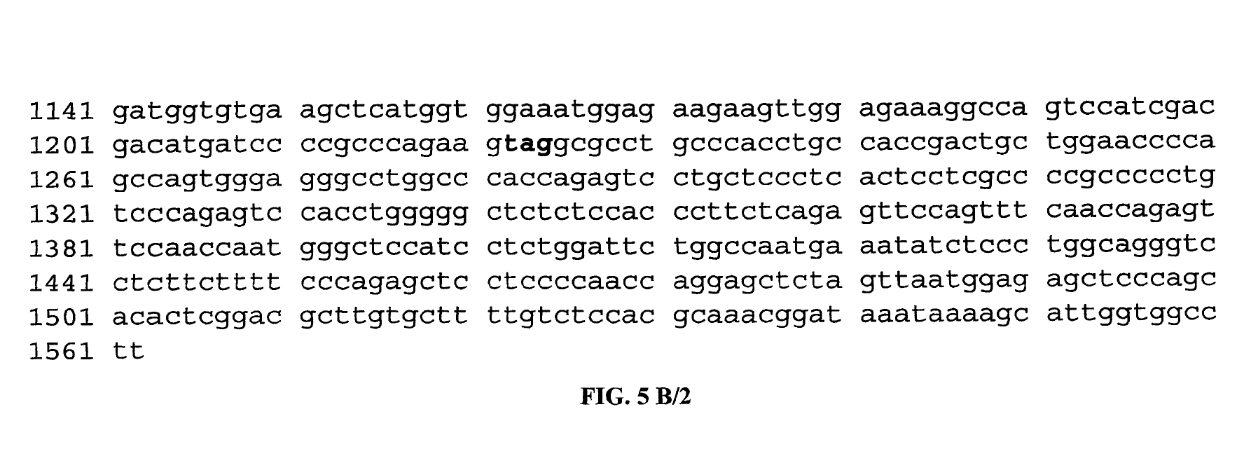

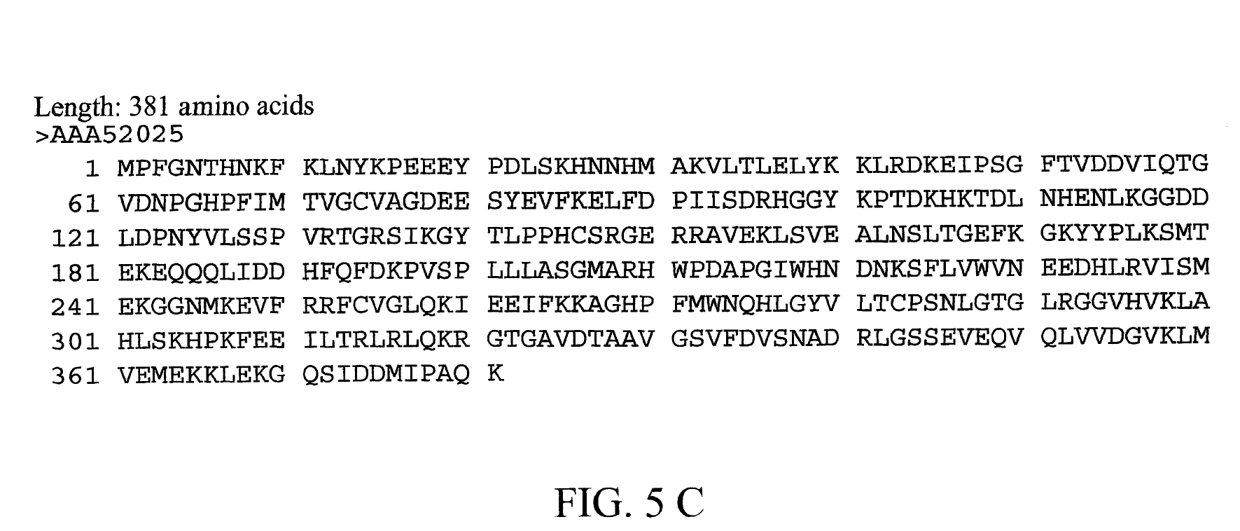

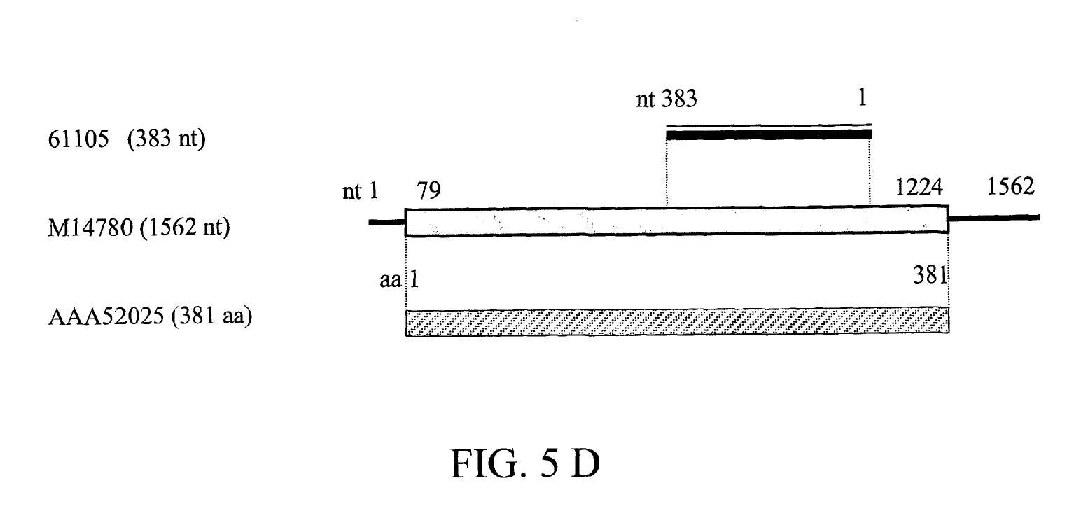

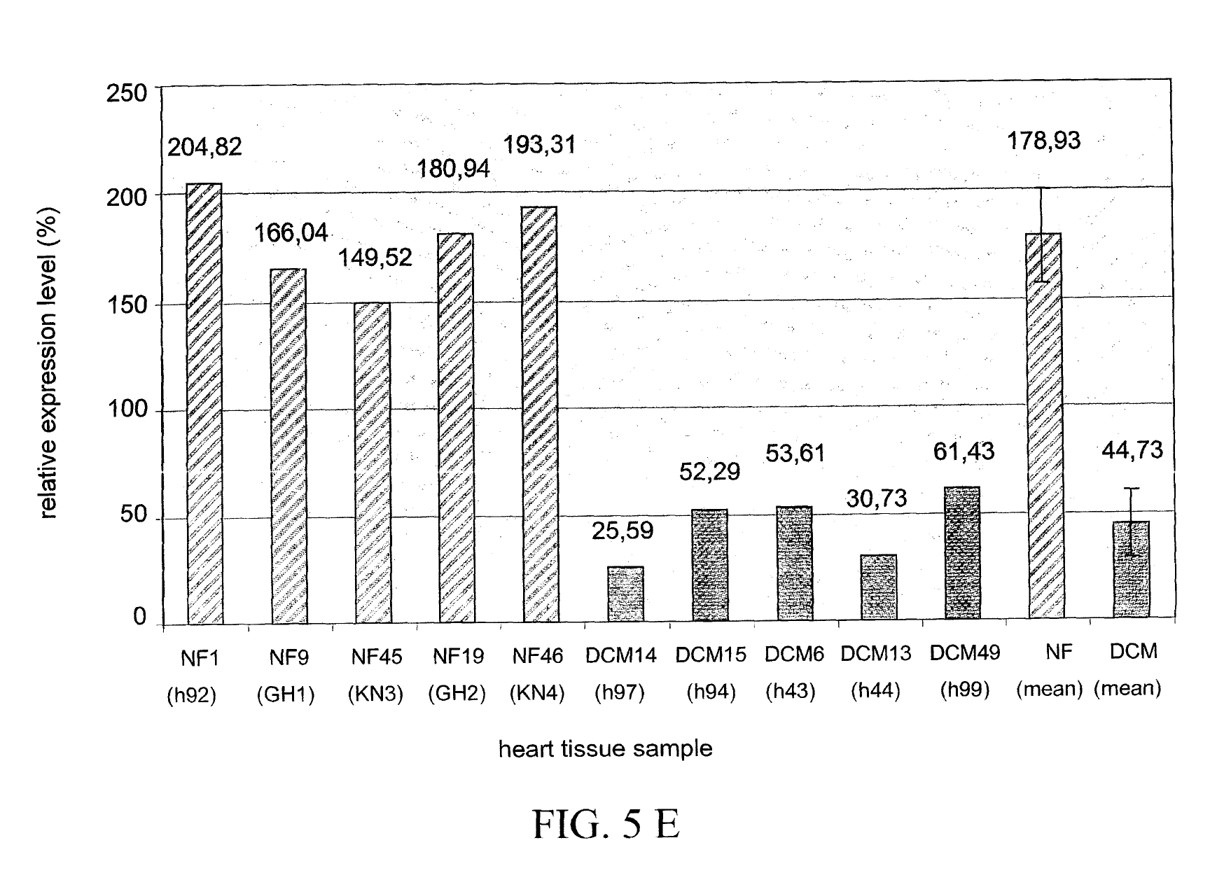

FIG. 5a shows the cDNA sequence of clone 61105 (corresponds to SEQ ID NO: 23).

FIG. 5b shows the sequence of the EST clone M14780 (corresponds to SEQ ID NO: 14). Start and stop codons are marked by bold letters, the sequence of 61105 is marked in italic letters.

FIG. 5c shows the putative amino acid sequence AAA52025 (corresponds to SEQ ID NO: 4).

FIG. 5d shows a schematic alignment of the cDNA fragment 61105 identified in SSH with its homologous Genbank entree and open reading frame of 381 amino acids (aa). Not to scale. Homology scores were determined using blast2 algorithm of NCBI:

61105-M14780: Expect=0.0, Identities=375/379 (98%), Positives=375/379 (98%), Gaps=1/379 (0%).

FIG. 5e: Two filters were hybridized sequentially with [α-33P]UTP labeled T3 transcripts from cDNA libraries prepared from mRNA of five control heart tissues and five DCM heart tissues as indicated. Experiments were normalized by adjusting the overall signal intensity of each hybridization to 100%, relative expression levels are given. Mean values and standard deviations were calculated form relative expression levels.

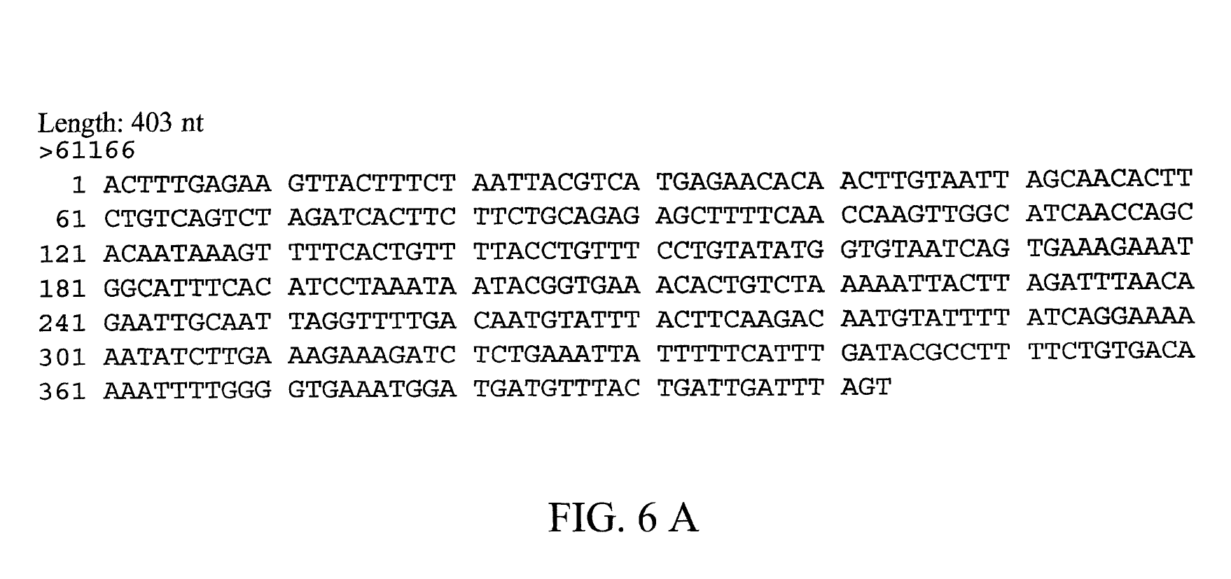

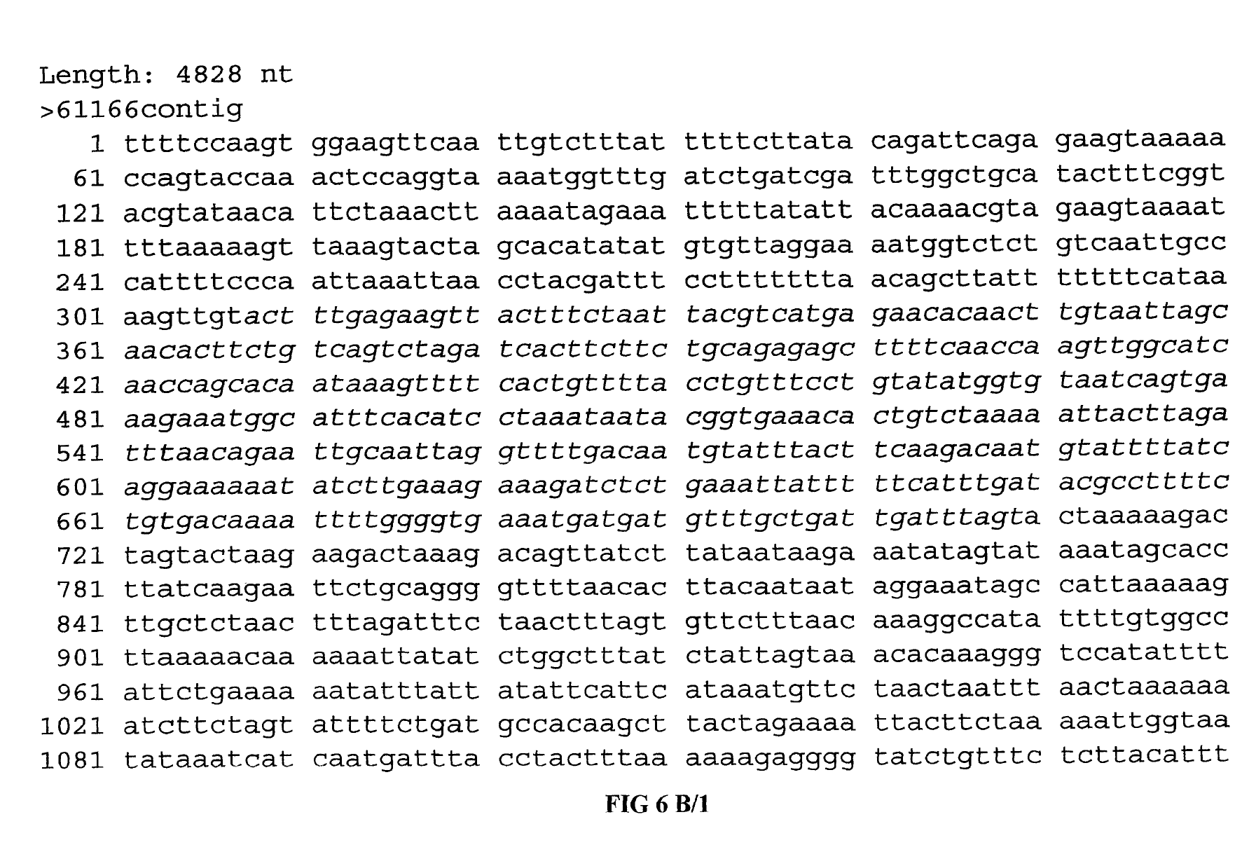

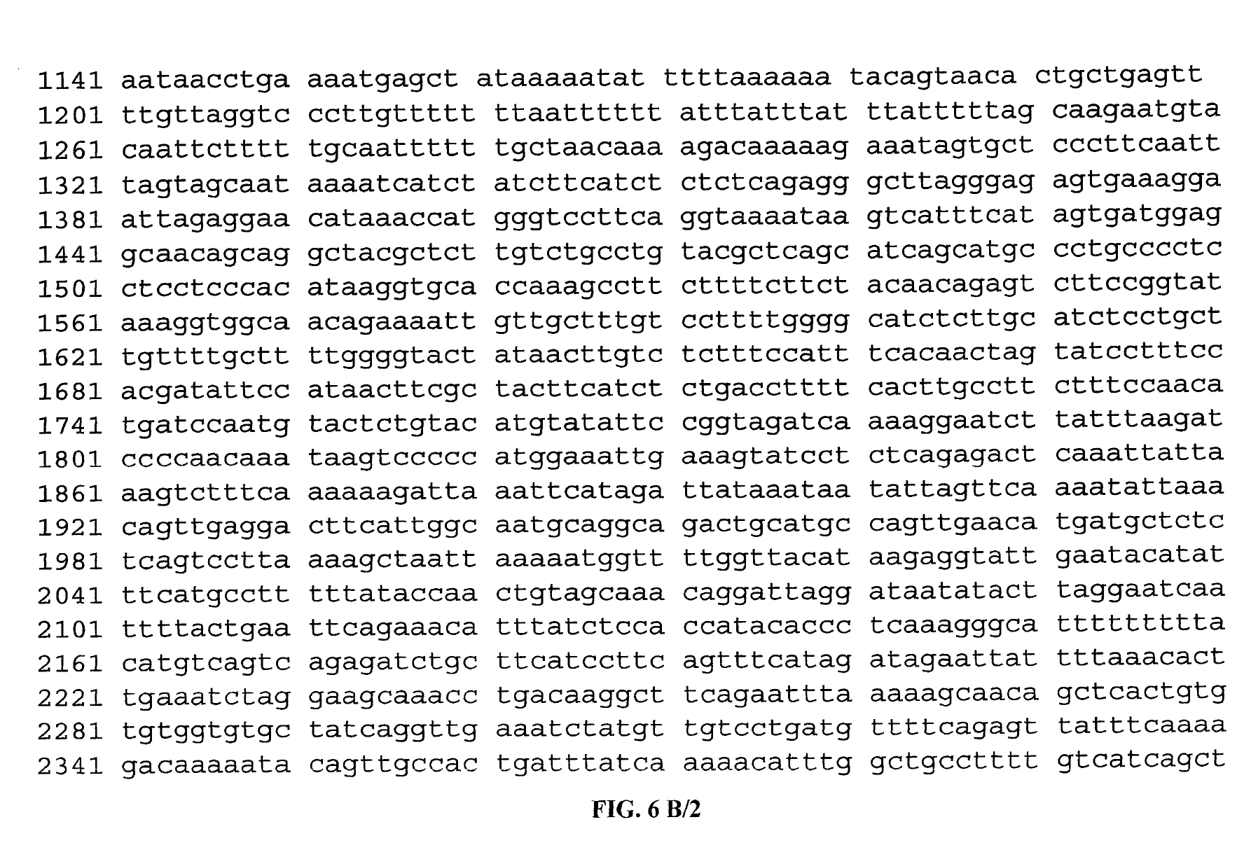

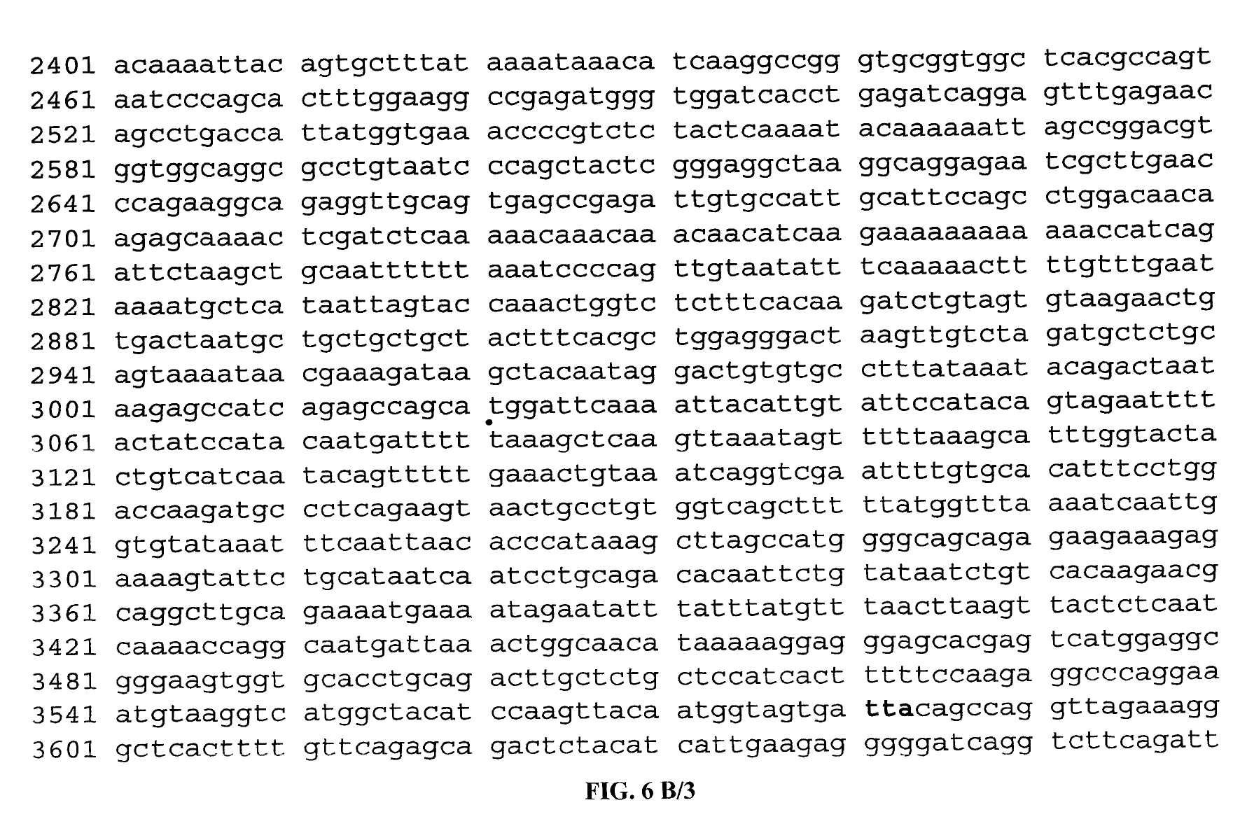

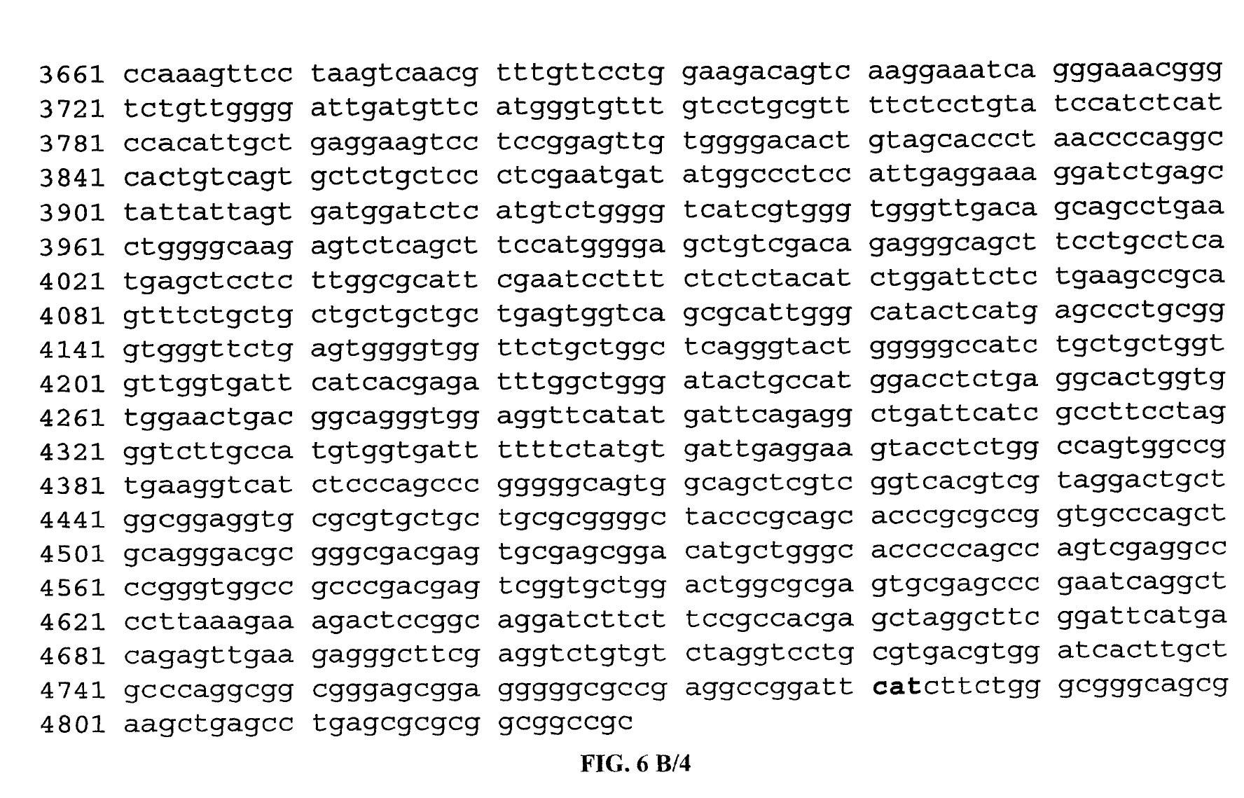

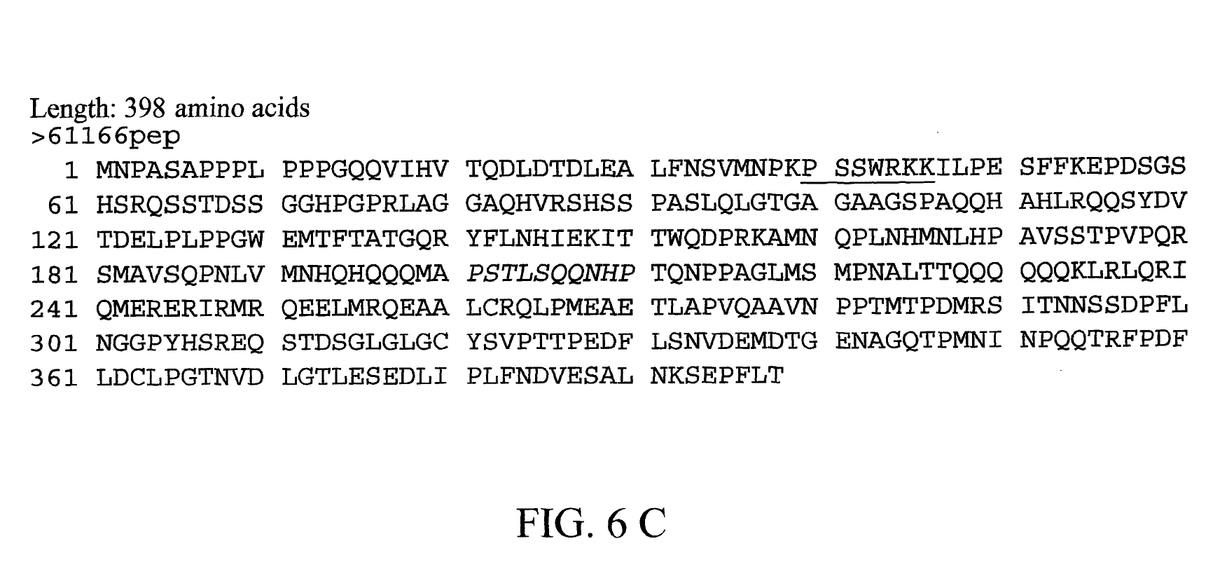

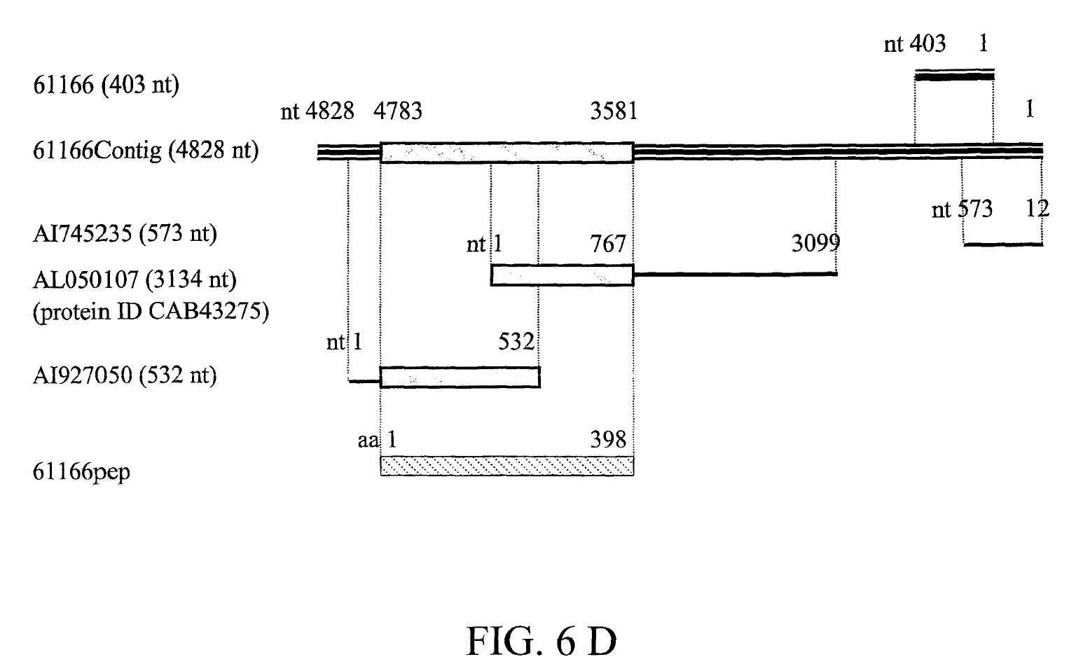

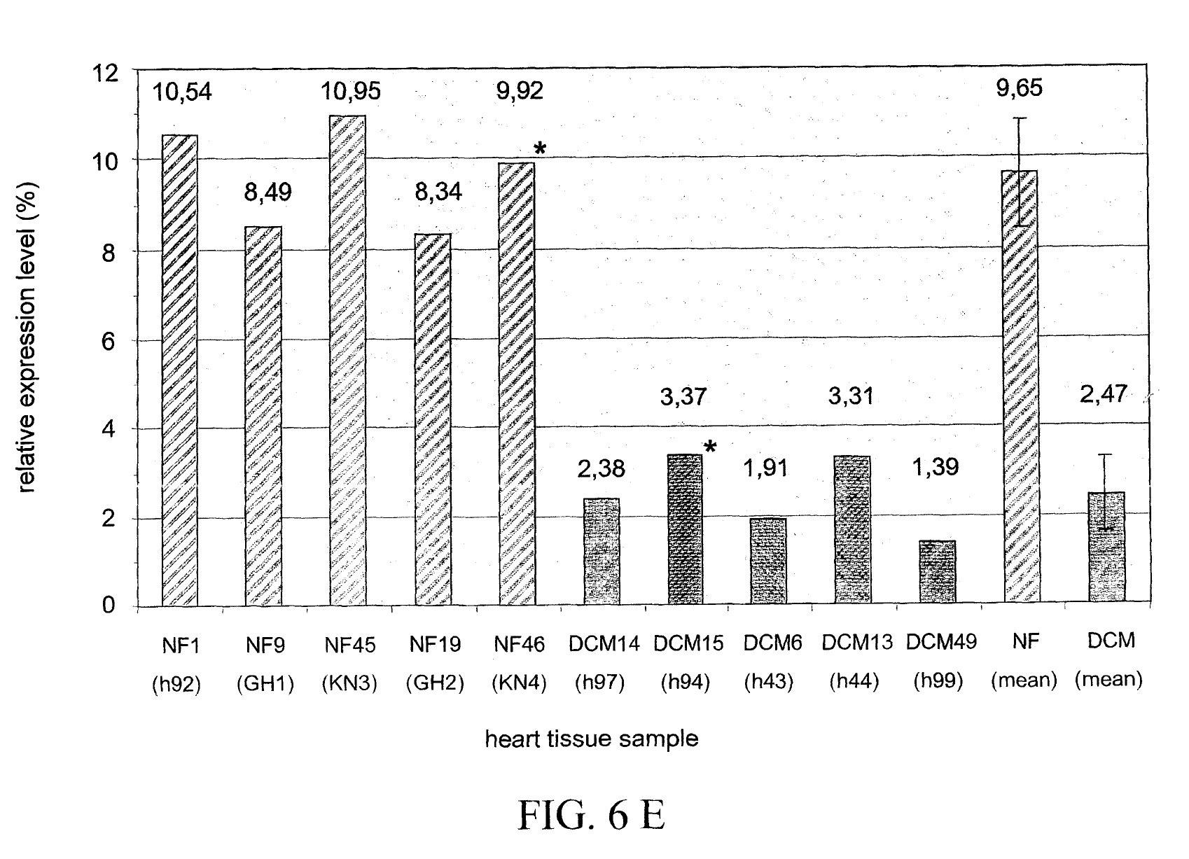

FIG. 6a shows the cDNA sequence of clone 61166 (corresponds to SEQ ID NO: 24).

FIG. 6b shows the sequence 611.66contig assembled from overlapping EST sequences, which are available from public databases (corresponds to SEQ ID NO: 15). Start and stop codons are marked by bold letters, the sequence of 61166 is marked in italic letters.

FIG. 6c shows the amino acid sequence of 61166pep (corresponds to SEQ ID NO: 5) Amino acids 40-46 of 61166pep encode a nuclear localization signal pattern 7 (PX1-3[KR][KR][KR], underlined) not present in human YAP65 (NP_{—}006097). Therefore this protein is expected to be located in the nucleus.

FIG. 6d shows a schematic alignment of the cDNA fragment 61166 identified in SSH with its overlapping contig of assembled EST sequences according to LabOnWeb (Compugen) analysis, accession numbers of homologous Genbank entrees and the longest open reading frame of 398 amino acids (aa). Not to scale. Homology scores were determined using blast2 algorithm of NCBI:

Contig-61166: Expect=0.0, Identities=401/403 (99%), Positives=401/403 (99%), Gaps=1/403 (0%)

Contig-AL050107: Expect=0.0, Identities=3058/3098 (98%), Positives=3058/3098 (98%)

Contig-A1927050: Expect=0.0, Identities=532/532 (100%), Positives=532/532 (100%)

Contig-A1745235: Expect=0.0, Identities=557/573 (97%), Positives=557/573 (97%), Gaps=1/573 (0%).

FIG. 6e: Two filters were hybridized sequentially with [α-33P]UTP labeled T3 transcripts from cDNA libraries prepared from mRNA of five control heart tissues and five DCM heart tissues as indicated. Experiments were normalized by adjusting the overall signal intensity of each hybridization to 100%, relative expression levels are given. Mean values and standard deviations are given on the right side. Asterisks mark samples used for SSH.

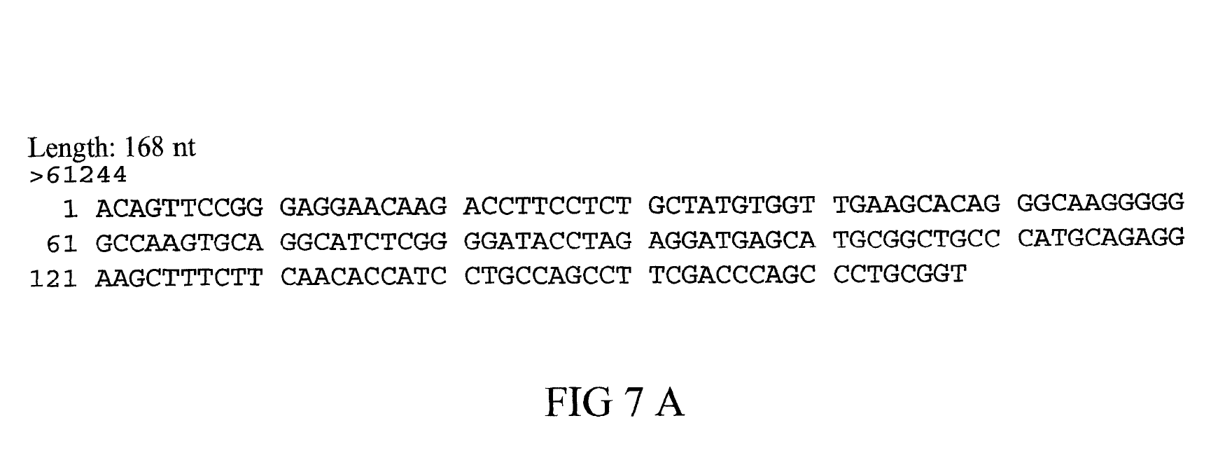

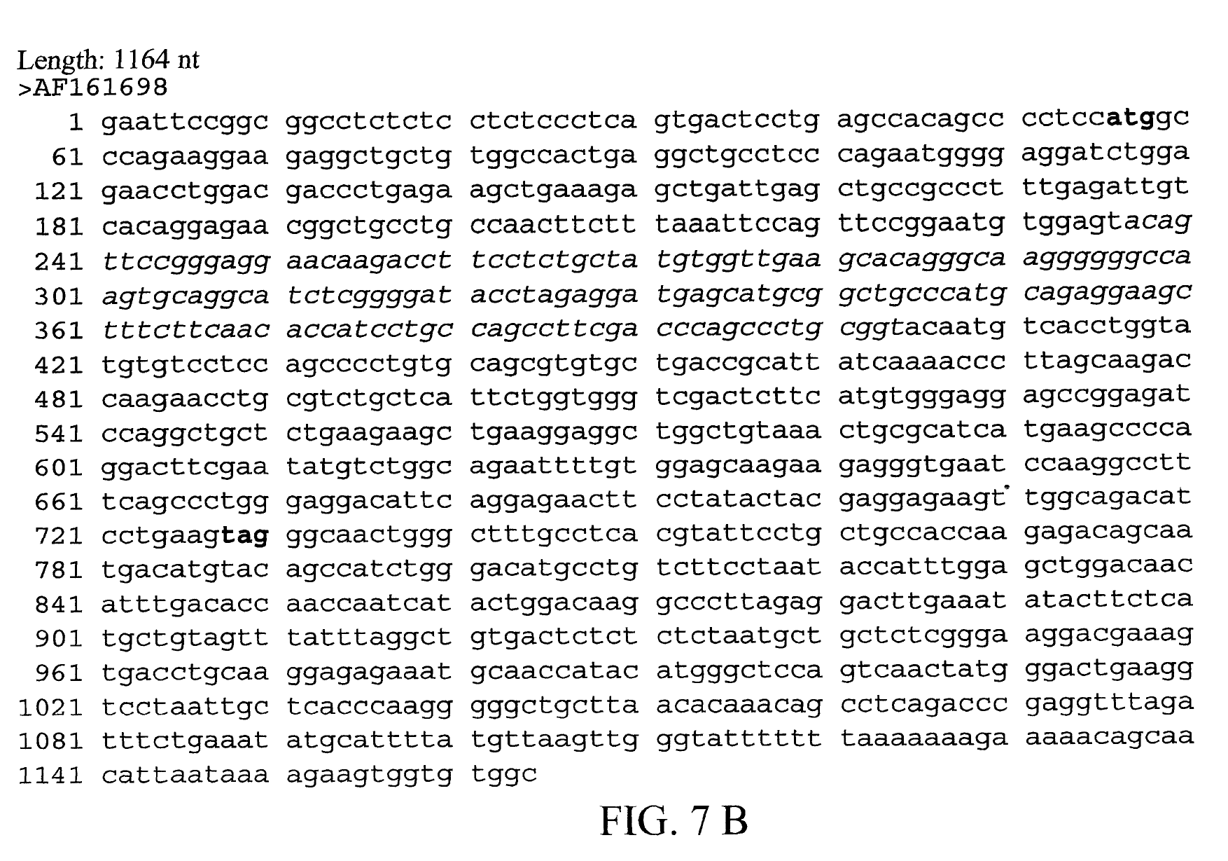

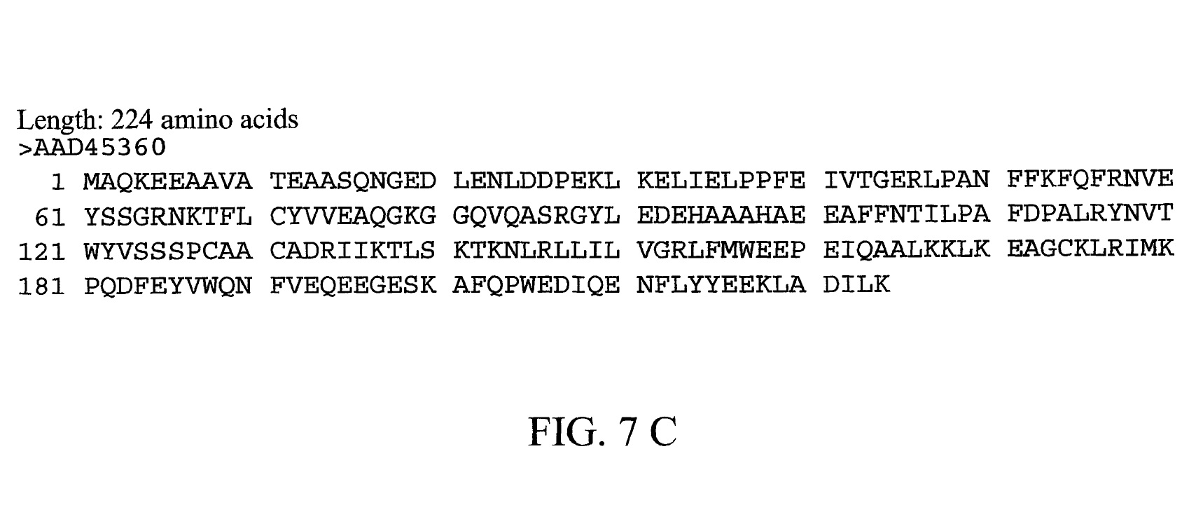

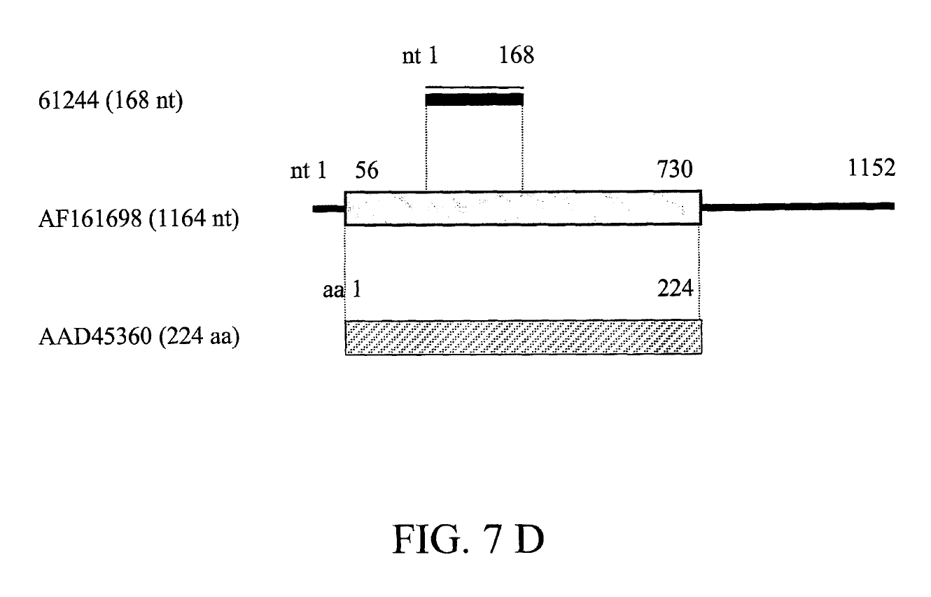

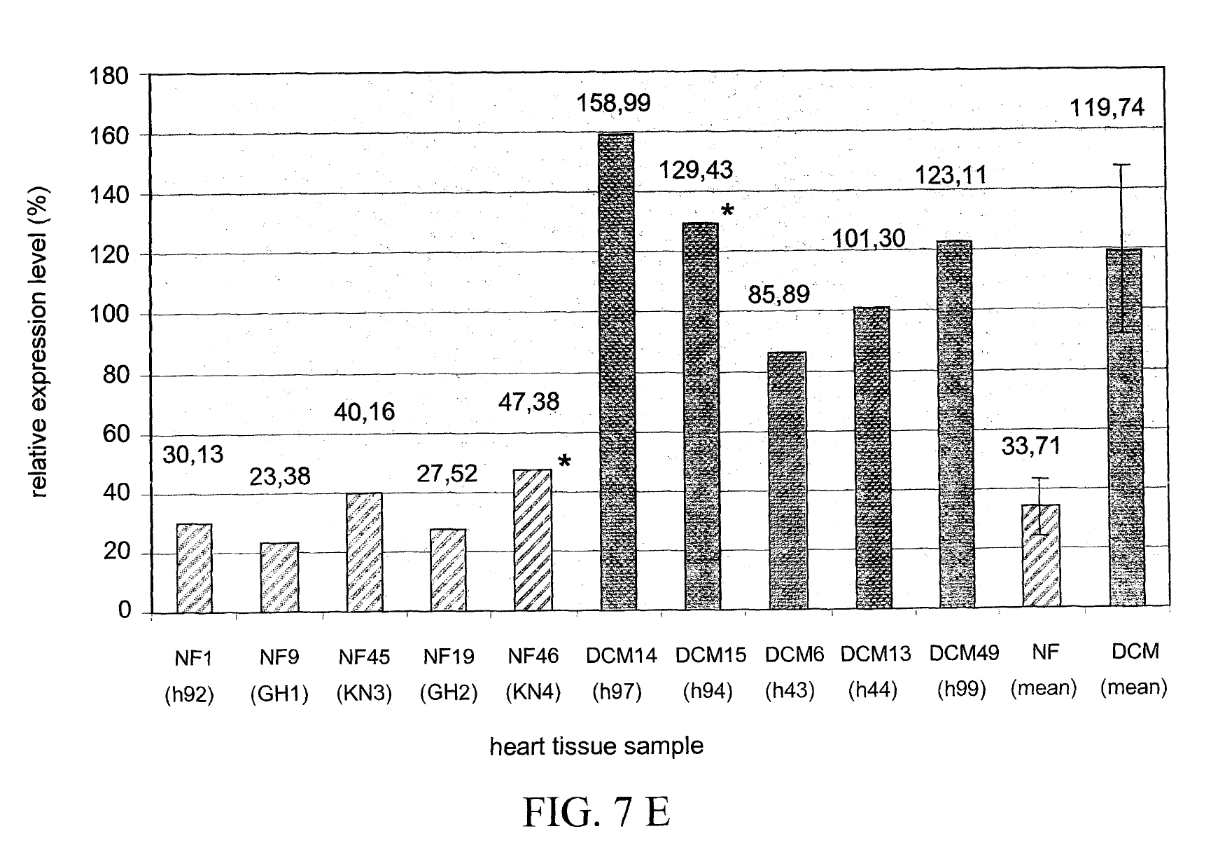

FIG. 7a shows the cDNA sequence of clone 61244 (corresponds to SEQ ID NO: 25).

FIG. 7b shows the sequence of the EST clone AF161698 (corresponds to SEQ ID NO: 16). Start and stop codons are marked by bold letters, the sequence of 61244 is marked in italic letters.

FIG. 7c shows the putative amino acid sequence AAD45360 (corresponds to SEQ ID NO: 6).

FIG. 7d shows a schematic alignment of the cDNA fragment 61244 identified in SSH with its homologous Genbank entree and open reading frame of 224 amino acids (aa). Not to scale. Homology scores were determined using blast2 algorithm of NCBI:

61244-AF161698: Expect=3e-86, Identities=168/168 (100%), Positives=168/168 (100%).

FIG. 7e: Two filters were hybridized sequentially with [α-33P]UTP labeled T3 transcripts from cDNA libraries prepared from mRNA of five control heart tissues and five DCM heart tissues as indicated. Experiments were normalized by adjusting the overall signal intensity of each hybridization to 100%, relative expression levels are given. Mean values and standard deviations were calculated form relative expression levels. Asterisks mark samples used for SSH.

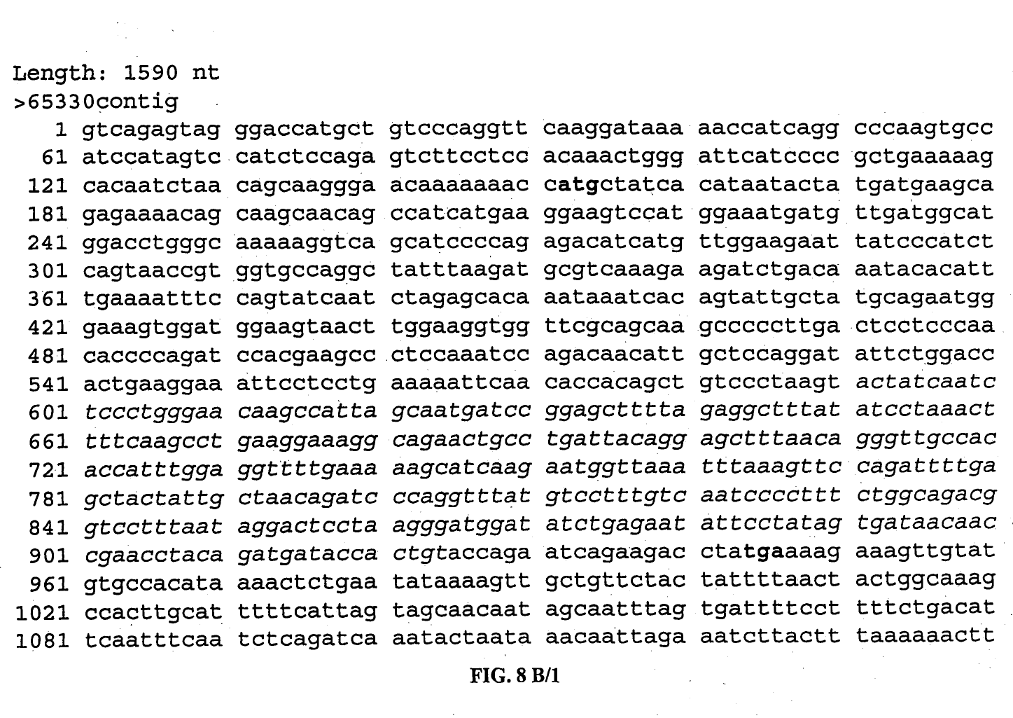

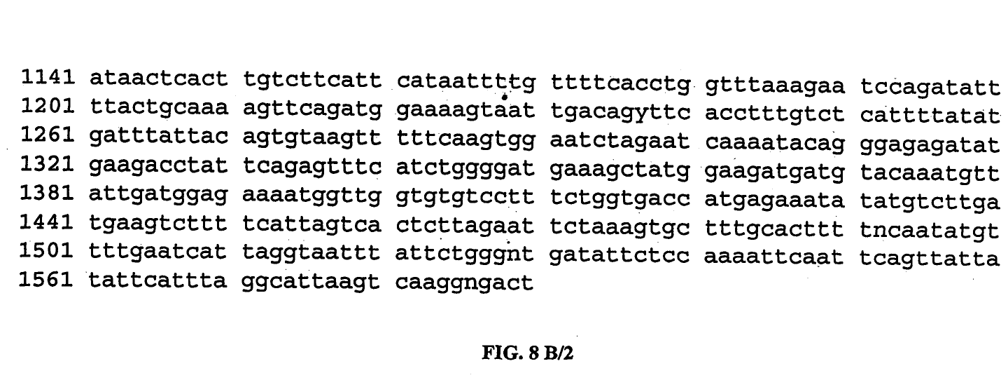

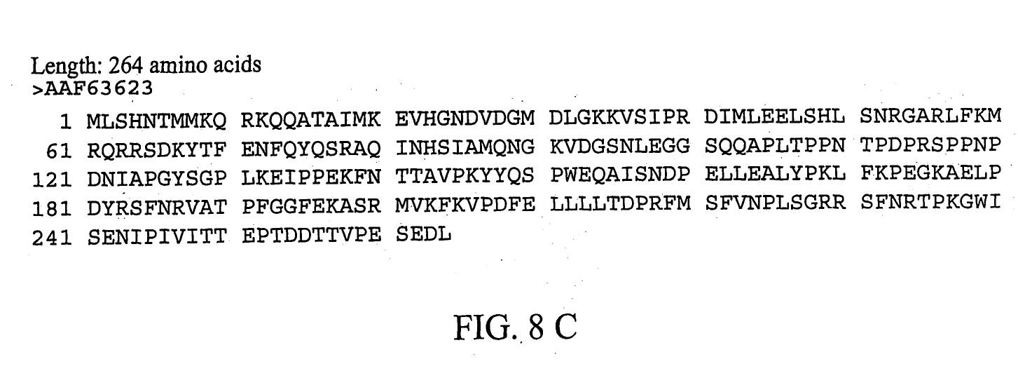

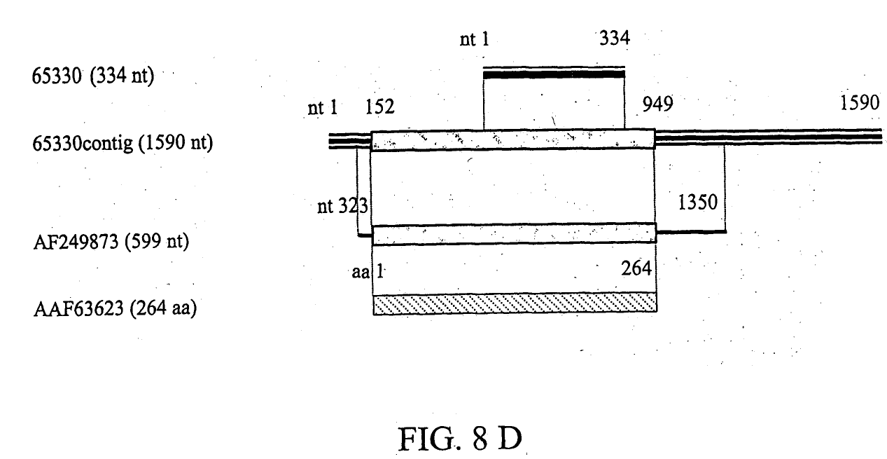

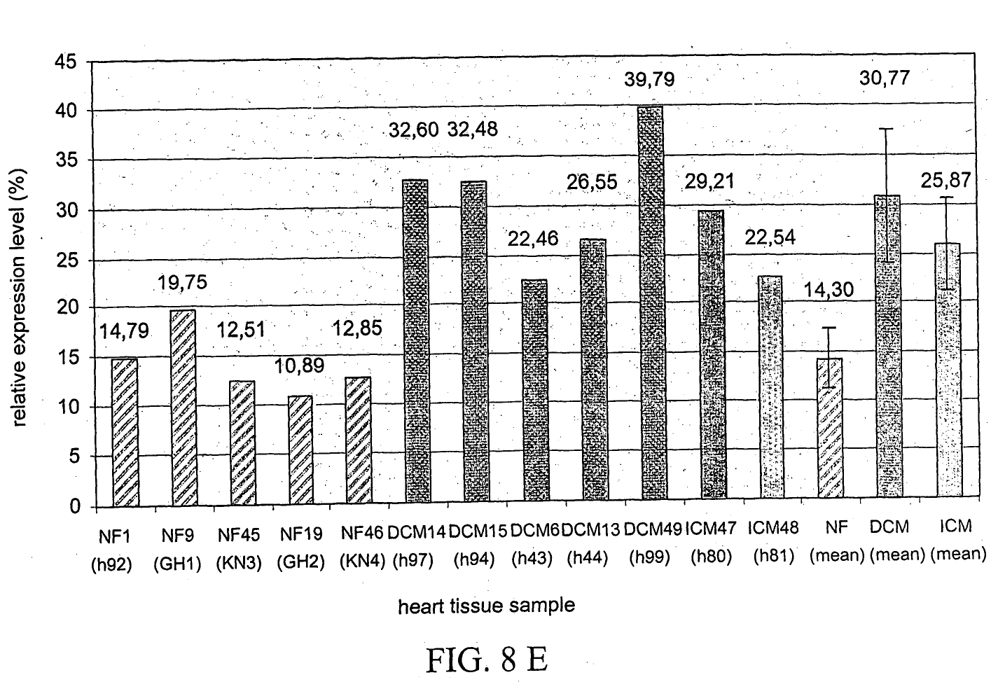

FIG. 8a shows the cDNA sequence of clone 65330 (corresponds to SEQ ID NO: 26).

FIG. 8b shows the contig of assembled EST sequences (corresponds to SEQ ID NO: 17). Start and stop codons are marked by bold letters, the sequence of 65330 is marked in italic letters.

FIG. 8c shows the putative amino acid sequence of clone 65330 (corresponds to SEQ ID NO: 7).

FIG. 8d shows a schematic alignment of the cDNA fragment 65330 identified in SSH with its overlapping contig of assembled EST sequences according to LabOnWeb (Compugen) analysis, accession numbers of homologous Genbank entree and the longest open reading frame of 264 amino acids (aa). Not to scale. Homology scores were determined using blast2 algorithm of NCBI:

Contig-65330:Expect=0.0, Identities=334/334 (100%), Positives=334/334 (100%)

Contig-AF249873: Expect=0.0, Identities=1020/1028 (99%), Positives=1020/1028 (99%).

FIG. 8e: Two filters were hybridized sequentially with [α-33P]UTP labeled T3 transcripts from cDNA libraries prepared from mRNA of five control, five DCM and two ICM heart tissues as indicated. Experiments were normalized by adjusting the overall signal intensity of each hybridization to 100%, relative expression levels are given.

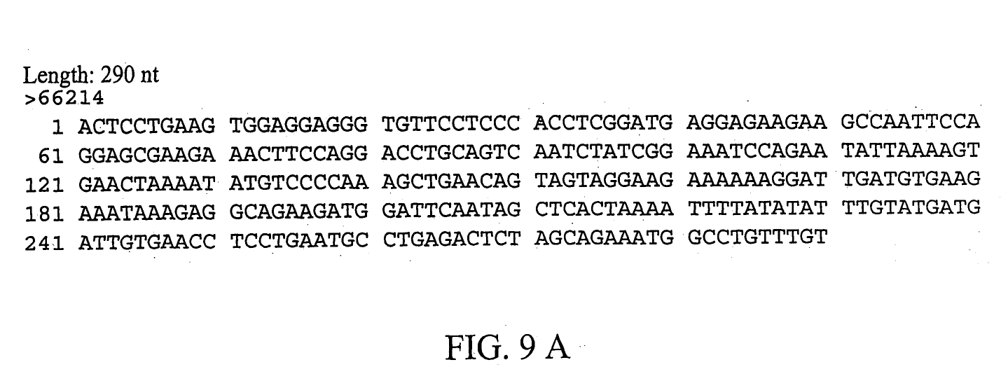

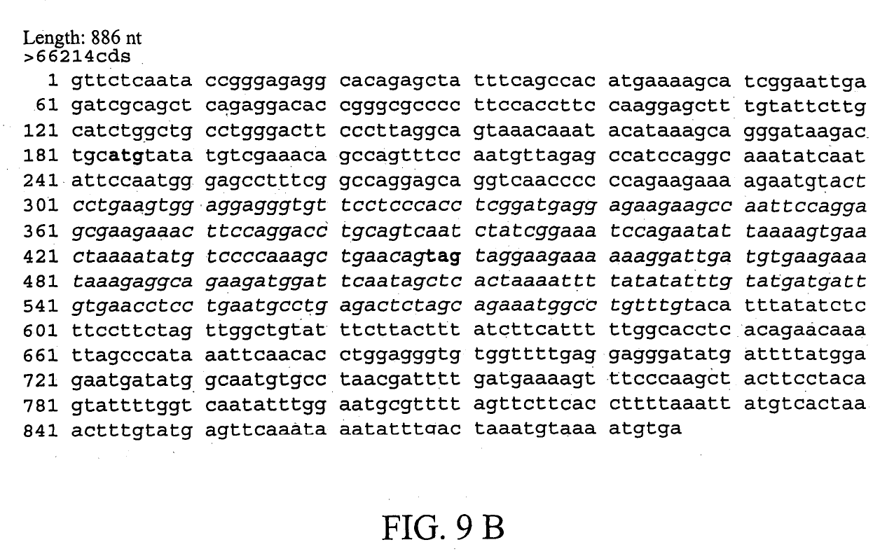

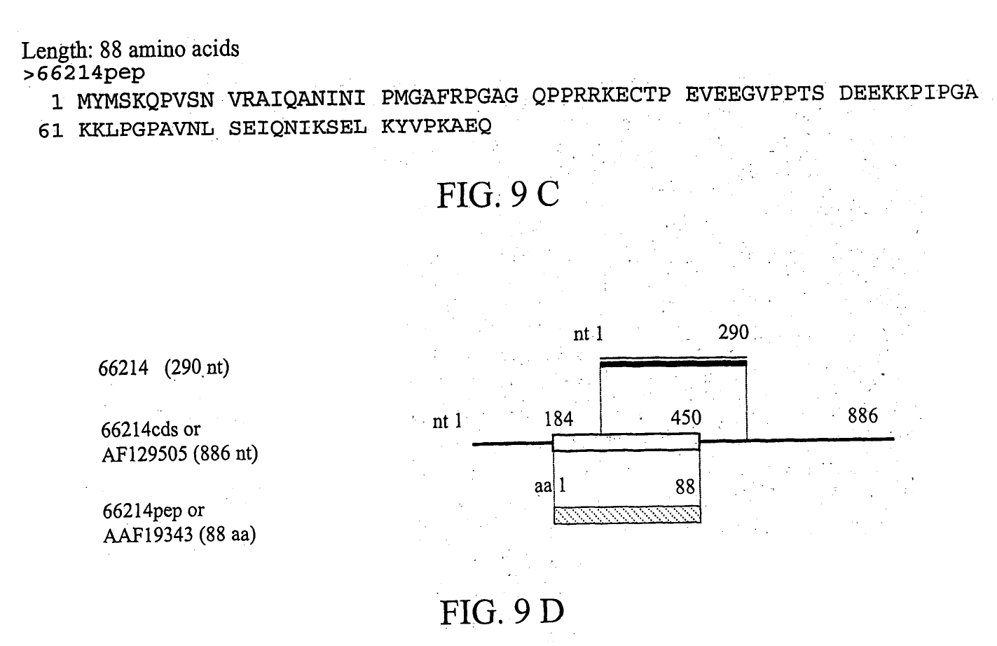

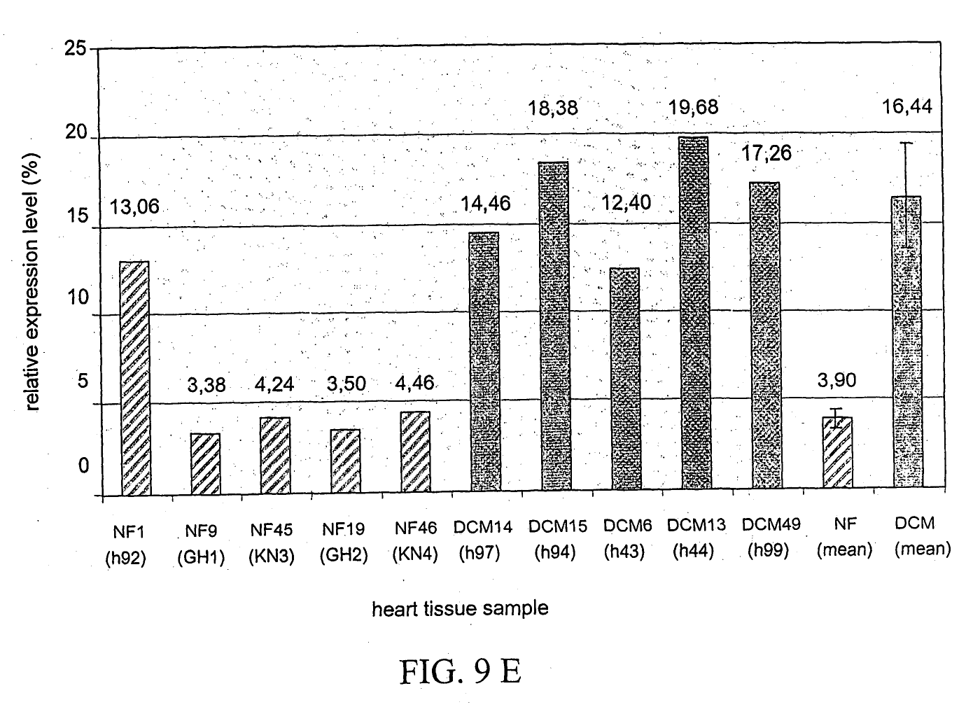

FIG. 9a shows the cDNA sequence of clone 66214 (corresponds to SEQ ID NO: 27).

FIG. 9b shows the sequence of the EST clone 66214cds (corresponds to SEQ ID NO: 18). The poly(A) signal is underlined, start and stop codons are marked by bold letters, the sequence of 66214 is marked in italic letters.

FIG. 9c shows the putative amino acid sequence 66214pep (corresponds to SEQ ID NO: 8).

FIG. 9d shows a schematic alignment of the cDNA fragment 66214 identified in SSH with the Genbank entree and open reading frame of 88 amino acids (aa). Not to scale. Homology scores were determined using blast2 algorithm of NCBI:

66214-AF129505: Expect=e-157, Identities=290/290 (100%), Positives=290/290 (100%).

FIG. 9e: Two filters were hybridized sequentially with [α-33P]UTP labeled T3 transcripts from cDNA libraries prepared from mRNA of five control and five DCM heart tissues as indicated. Experiments were normalized by adjusting the overall signal intensity of each hybridization to 100%, relative expression levels are given. NF1 was not taken into account for calculation of mean values and standard deviations.

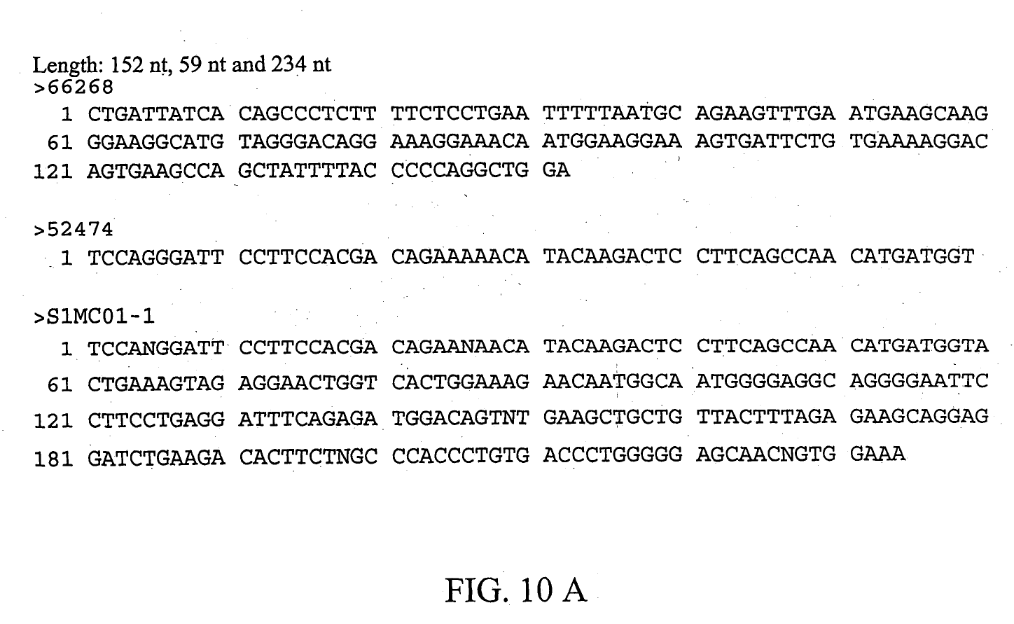

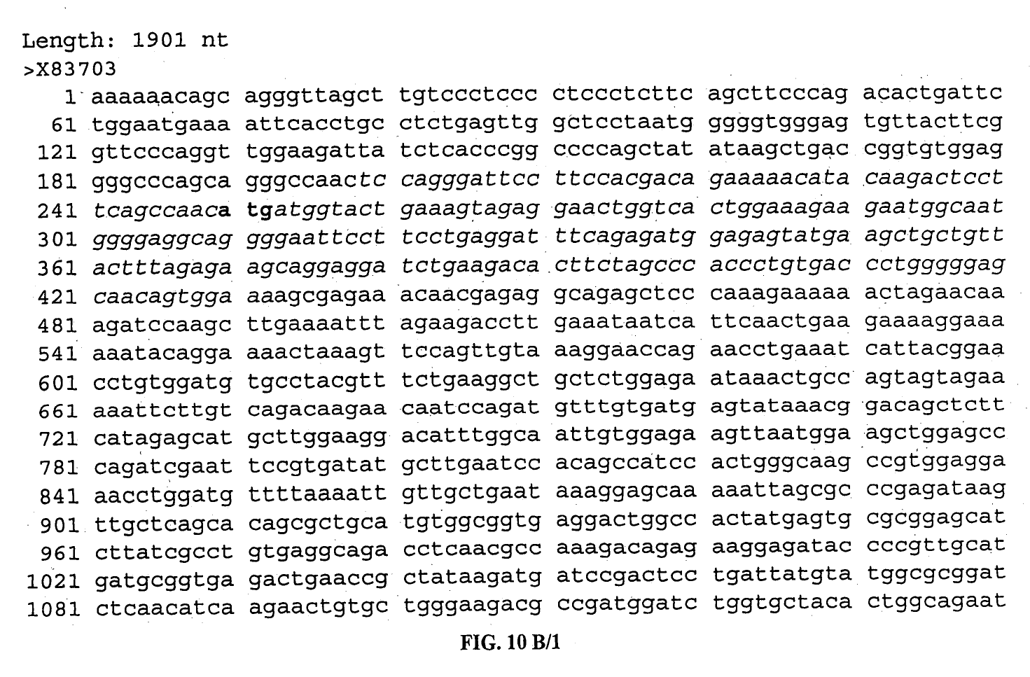

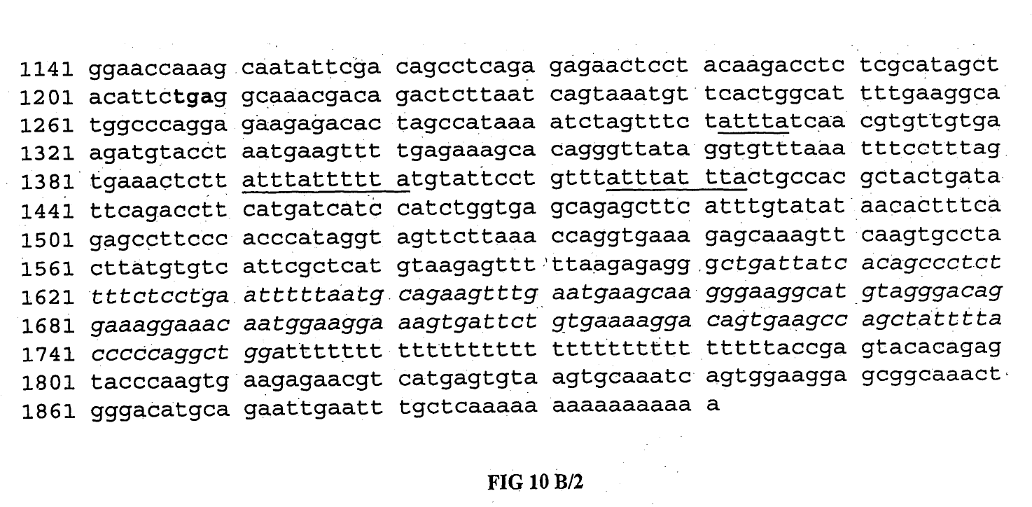

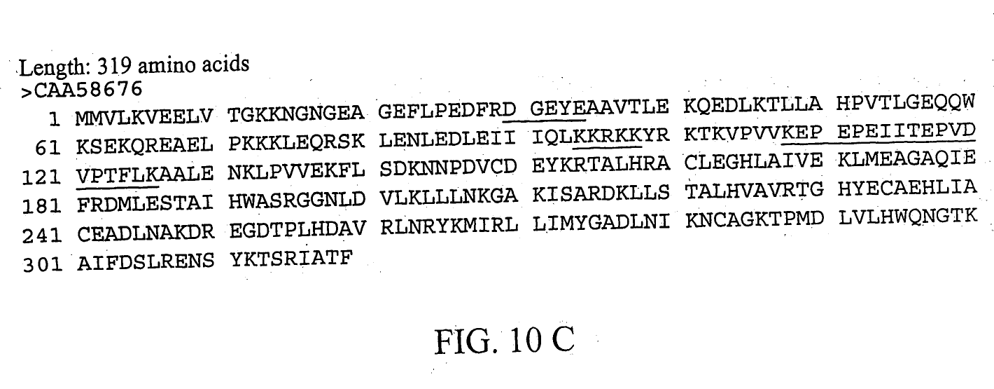

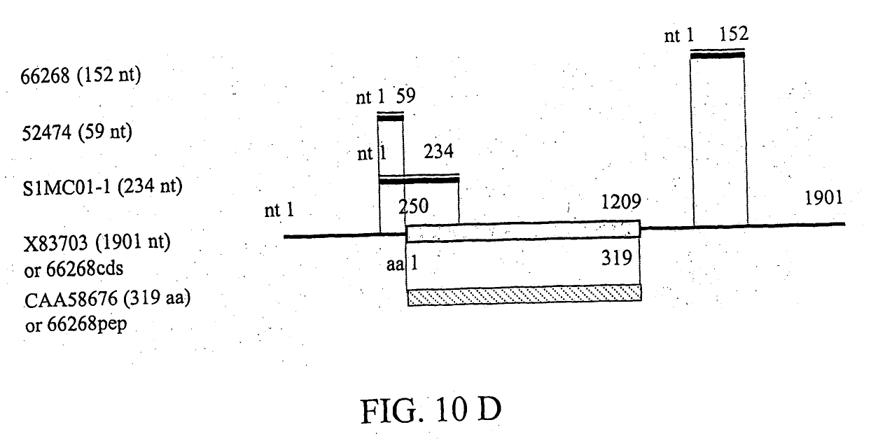

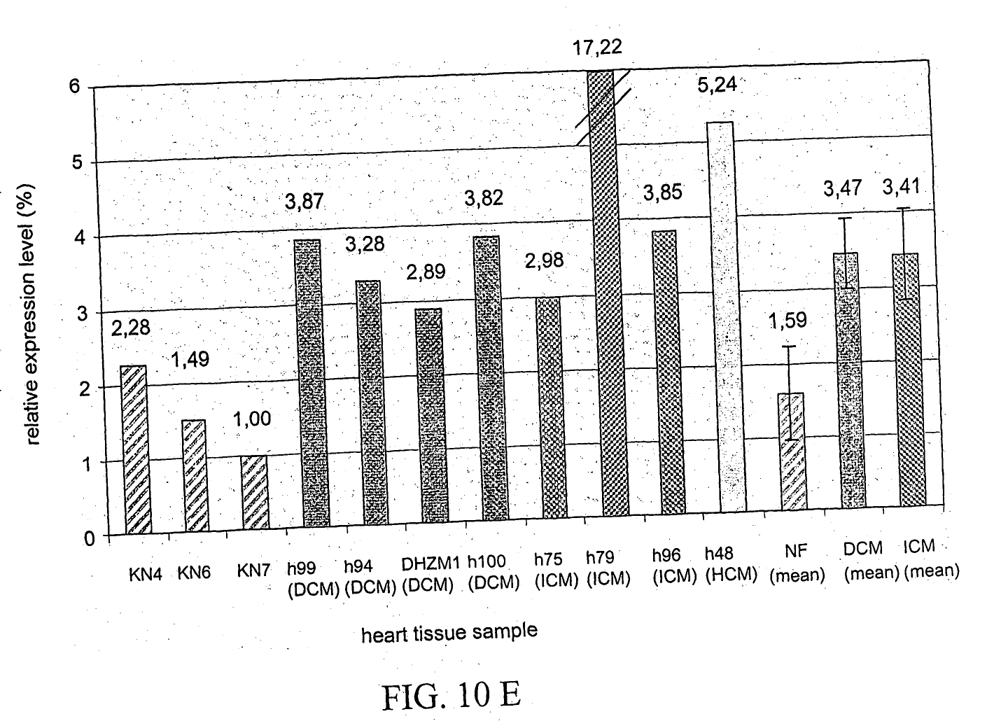

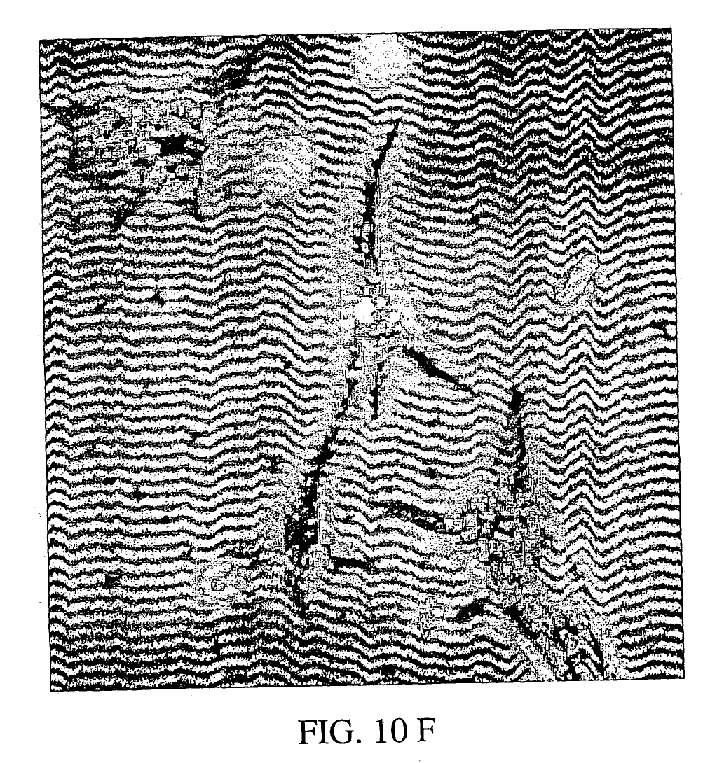

FIG. 10a shows the cDNA sequence of clone 66268 (corresponds to SEQ ID NO: 28), 52474 (corresponds to SEQ ID NO: 29) and S1MC01-1 (corresponds to SEQ ID NO: 30).

FIG. 10b shows the sequence of the EST clone X83703 (corresponds to SEQ ID NO: 19). Start and stop codons are marked by bold letters, the sequences of 66268 and S1MC01-1 are marked in italic letters. Multiple AU-rich mRNA decay elements are present in the 3′-noncoding region (underlined).

FIG. 10c shows the putative amino acid sequence CAA58676 (corresponds to SEQ ID NO: 9). Amino acids 94-97 of 66268 encode a nuclear localization signal pattern 4 ([KR][KR][KR][KR]). The protein is described to be located in the nucleus. Moreover, a PEST-rich region (aa 108-126), a tyrosine phosphorylation site (aa 33) and a domain containing four tandem ankyrin-like repeats (aa 152-183) have also been found.

FIG. 10d shows a schematic alignment of the cDNA fragments identified in SSH and FDD, respectively with their homologous Genbank entree and the open reading frame of 3.19 amino acids (aa). Not to scale. Homology scores were determined using blast2 algorithm of NCBI:

66268-X83703: Expect=9e-77, Identities=152/152 (100%), Positives 152/152 (100%)

52474-X83703: Expect=6e-23, Identities=59/59 (100%), Positives=59/59 (100%)

S1MC01-1-X83703: Expect=e-115, Identities=227/234 (97%), Positives=227/234 (97%).

FIG. 10e shows RNA samples prepared from three control, four DCM, three ICM and one HCM heart tissue have been compared by fluorescence differential display using the primer combination. [T7]T12MC and [M13r]ARP1 (with the arbitrary sequence CGACTCCAAG). The relative expression was calculated using ImageQuant Software and the lowest value set to 1 as reference for all values. Mean values and standard deviations were calculated from all NF and DCM samples, as well as from ICM75 and ICM96.

FIG. 10f depicts the recombinant over expression of a 66268-YFP fusions protein in pCMs. The pCMs were transfected with an expression plasmid for a 66268-YFP fusions protein and stimulated with Phenylephrine (100 μM). The YFP signal was detected with a fluorescence microscope (Axiovert 100S, Zeiss (Jena); YFP filter set, AF-Analysetechnik (Tübingen)) in combination with a digital camera (LAS-1000, Fuji; AIDA-software, Raytest).

### EXAMPLES

The following examples illustrate the invention. These examples should not be construed as limiting: the examples are included for purposes of illustration and the present invention is limited only by the claims.

### Example 1

1. Isolation of Total RNA from Heart Tissue

Total RNA was isolated from tissue of explanted hearts of left ventricle of human non-failing and DCM patients, which are listed in TABLE 1, respectively, according to the protocol of Chomczynski and Sacchi with some minor modifications. 0.5 g tissue were disrupted using a mortar and pestle and grinded under liquid nitrogen. The suspension of tissue powder and liquid nitrogen was decanted into a cooled 50 ml polypropylene tube and nitrogen allowed to evaporate completely without thawing the sample. After addition of 10 ml solution D (4 M guanidinium thiocyanate, 25 mM sodium citrate pH 7.0, 0.5% sodium-N-lauroyl-sarcosinat, 0.1 M 2-mercaptoethanol) the sample was homogenized immediately using a rotor-stator homogenizer (Ultra-Turrax T8, IKA Labortechnik) for 60 s at maximum speed. The sample was mixed with 1 ml 2 M NaOAc pH 4.0, 10 ml phenol (water saturated, pH 4.5-5) and 2 ml chloroform/isoamylalcohol (49/1). After incubation on ice for 15 min and centrifugation at 10000 g for 30 min at 4° C. the aqueous phase was transferred to a fresh 50 ml polypropylene tube. RNA was precipitated with 1 vol isopropanol at −20° C. for at least one hour. After centrifugation at 10000 g for 30 min at 4° C. the RNA pellet was redissolved in 5 ml Solution D and precipitated again with 1 vol isopropanol as described. The pellet was washed with cold 75% EtOH and dried at RT for 15 min. To completely dissolve RNA 500 μl DEPC-treated water were added and the sample was incubated at 60° C. for 10 min, final storage was at −80° C. An aliquot was used for quantification by A₂₆₀ measurement and separation on a formaldehyde agarose gel (Sambrook et al.) to check integrity and size distribution.

TABLE 1
Human heart samples

| | | | | | | explantation |
|---|---|---|---|---|---|---|
| ID heart | ID library | diagnosis | sex | age | medication | date |
| Normal | | | | | | |
| controls | | | | | | |
| GH1 | NF9 | cerebral hemorrhage | f | 53 | unknown | May 18, 1995 |
| GH2 | NF19 | unknown | m | | unknown | |
| h92 | NF1 | (suspicion on hepatitis B) | f | 50 | unknown | Jul. 20, 1994 |
| KN3 | NF45 | intracranial pressure at | f | 41 | Minirin, Dopamin, | Aug. 30, 1996 |
| | | astrocytoma IV | | | Rocephin, Dexamethason | |
| KN4 | NF46 | traumatic brain disease | m | 33 | Arterenol, KCl | Jun. 8, 1997 |
| KN6 | — | unknown | | | unknown | Jul. 6, 1997 |
| KN7 | — | unknown | | | unknown | Jan. 2, 1998 |
| DCM | | | | | | |
| samples | | | | | | |
| h43 | DCM6 | DCM | f | 54 | Digitalis, diuretics, | Apr. 24, 1990 |
| | | | | | nitrates, ACEI | |
| h44 | DCM13 | DCM, Z.n. myocarditis | m | 22 | unknown | May 8, 1990 |
| h94 | DCM15 | DCM | m | 16 | Digitalis, ACEI, nitrate, | Nov. 3, 1994 |
| | | | | | catecholamines, diuretics | |
| h97 | DCM14 | DCM | m | 62 | Digitalis, diuretics, ACEI, | Jan. 4, 1995 |
| | | | | | Amiodaron, Marcumar | |
| h99 | DCM49 | DCM | m | 64 | Digitalis, diuretics, ACEI, | May 17, 1995 |
| | | | | | Amiodaron, Marcumar, | |
| | | | | | nitrate | |
| h100 | — | DCM | | | unknown | Sep. 20, 1996 |
| DHZM1 | — | DCM | m | 53 | unknown | |
| ICM | | | | | | |
| samples | | | | | | |
| h75 | — | ICM | | | unknown | Oct. 5, 1992 |
| h79 | — | ICM | | | unknown | Apr. 20, 1993 |
| h80 | ICM47 | ICM | | | unknown | Jun. 10, 1993 |
| h81 | ICM48 | ICM | | | unknown | Jun. 17, 1993 |
| h96 | — | ICM | m | 39 | Digitalis, ACEI, | Dec. 13, 1994 |
| | | | | | Amiodaron, Marcumar | |
| HCM | | | | | | |
| samples | | | | | | |
| h48 | — | non-obstructive HCM | m | 37 | unknown | Jan. 8, 1991 |

2. Isolation of Poly(A) RNA from Total RNA

Poly(A) RNA was isolated from 300 μg total RNA (see 1.) using the PolyA Quick mRNA Isolation Kit (Stratagene) according to the manufacturers protocol. Purified mRNA was dissolved in 30 μl RNase-free water (Stratagene), quantified and analyzed on a formaldehyde agarose gel as described (see 1.).

3. Suppression Subtractive Hybridization (SSH)

3.1 Construction of a Subtracted Library

2 μg of tester mRNA and 2 μg of driver mRNA were used to construct a subtracted and normalized cDNA library using the PCR-Select cDNA Subtraction Kit and Advantage cDNA-Polymerase Mix (Clontech) according to the manufacturers protocol. In general, two libraries were constructed for each tester and driver combination, since only transcripts can be identified that are over-represented in the tester mRNA.

Both, the subtracted and non-subtracted cDNA population were analyzed on an agarose gel as described (Clontech) and transferred onto Zeta-Probe GT nylon membrane (BioRad) by capillary forces (Sambrook et al.). The membrane was UV crosslinked in a Stratalinker 2400 (Stratagene).

To analyze the subtraction efficiency the membrane was hybridized with a Digoxigenin-labeled probe synthesized from a housekeeping gene using the Dig-DNA Labeling and Detection Kit (Roche). For probe synthesis a 451 bp fragment of human GAPDH was amplified from 0.5-1 μg cDNA of a NF heart library (see 5.1.) in a 100 μl PCR reaction with the primer pair provided by the PCR-Select cDNA Subtraction Kit (Clontech). 100 ng of gel purified (QIAquick Gel Extraction Kit, Qiagen) GAPDH cDNA fragment then were used for Dig-labeling. The hybridized membrane was exposed to a X-ray film (X OMAT AR, Kodak) for 15 min. Only subtractions, where the GAPDH signal intensity of the subtracted cDNA population was at least four fold lowered compared to the corresponding non-subtracted cDNA-population, were selected for further analysis. 17 μl of the subtracted sample were purified using a PCR Purification Kit (Qiagen) and eluted in 20 μl ddH₂O (Gibco BRL).

For addition of 3′-A overhangs 15.7 μl of the purified subtracted cDNA sample was incubated in the presence of PCR buffer, 1.5 U Taq DNA polymerase (APB) and 0.2 mM dATP for 11 min at 72° C. 3 μl of the sample was ligated into the pGEM-T easy vector (Promega) and competent E. coli cells were transformed as described by the manufacturer.

3.2 Amplification of Subtracted cDNA Clones

Subtracted cDNA clones were grown over night at 37° C. in 96 well microplates filled with 100 μl LB medium (Sambrook et al.) and supplemented with 10 μg/ml Amp. 1 μl of the bacterial culture then was transferred into 99 μl PCR premix (1×PCR buffer, 2.5 U Taq DNA polymerase (APB), 0.2 mM dNTP) and directly amplified using the nested primer pair 1 and 2R provided by the PCR-Select cDNA Subtraction Kit (Clontech). Best results were obtained with 27 cycles and an annealing and polymerization temperature of 68° C. The size distribution of PCR-products was analyzed on an 1% agarose gel (Sambrook et al.). Bacterial cultures were mixed with glycerol to a final concentration of 20% and stored at −80° C.

4. Fluorescence Differential Display (FDD)

4.1 DNaseI Digestion

Total RNA (see 1.) was digested using the MessageClean-Kit (GeneHunter) according to the manufacturers protocol.

4.2 Reverse Transcription

Four degenerated primer pools [T7]-T₁₂MA, [T7]-T₁₂MC, [T7]-T₁₂MG and [T7]-T₁₂MT anchoring to poly(A) tails of mRNAs were used, where M is the degenerated position (a mixture of A, C, G). A 17 nt T7 RNA polymerase promoter-derived site (ACGACTCACTATAGGGC) is incorporated which allows the generation of an antisense transcript. For each RNA sample four separate reactions were performed. 200 ng of DNA-free RNA (see 4.1.) was denatured for 5 min at 70° C. in the presence of 0.2 μM anchor primer [T7]-T₁₂MX and 20 U rRNasin (Promega). After addition of RT buffer (Gibco), 10 mM DTT, 25 μM dNTP and 200 U SuperscriptII RTaseII (Gibco) on ice, the reaction with a final volume of 20 μl was performed for 5 min at 42° C. and 1 h at 50° C. The reaction was stopped by heating 15 min at 70° C.

4.3 PCR

Resulting cDNAs (see 4.2.) were reamplified in the presence of the same anchor primer labeled with Cy5 and a second primer with 10 nt of arbitrary chosen sequence. A 16 nt segment of the M13 universal reverse (−48) 24mer priming sequence (ACAATTTCACACAGCA) is incorporated in the arbitrary primer [M13r]-ARPX₁₀ for direct sequencing.

1 μl of reverse transcription sample (see 4.2.) was mixed on ice with 1×PCR buffer (Qiagen), 3.75 mM MgCl₂, 0.35 μM Cy5-[T7]-T₁₂MX, 0.35 μM [M13r]-ARPX₁₀, 50 μM dNTP and 0.5 U Taq polymerase (Qiagen) in a final volume of 20 μl. PCR was run in a Peltier Thermal Cycler PTC 200 (MJ Research) under the following conditions: 2 min 95° C., [15 s 92° C., 30 s 50° C., 2 min 72° C.]₄, [15 s 92° C., 30 s 60° C., 2 min 72° C.]₂₅, 7 min 72° C., 4° C.

4.4 Electrophoresis on a 6% Deanaturing Polyacrylamide Gel

The PCR sample (20 μl, see 4.3.) was mixed with 6 μl gel loading dye (95% formamide, 20 mM EDTA, 0.005% BPB), denatured for 2 min at 80° C. and separated on a standard sequencing gel (6% polyacrylamide/8.3 M urea) at 55 W for 3 h. The gel was dried on Whatman 3MM paper and fluorescence signals read at 635 nm on a Storm fluorimager (Molecular Dynamics). Data analysis was performed using ImageQuant Software (Molecular Dynamics) as described below (see 6.3.).

4.5 Recovery of PCR Fragments from the Sequencing Gel

Individual bands of interest (see 4.4.) were cut out of the gel with a scalpel. The gel slice attached to Whatman paper was soaked for 1 h at 37° C. (300 rpm) in 100 μl buffer EB (Qiagen) and incubated at 4° C. over night. Supernatant was purified using the QIAquick PCR purification Kit (Qiagen) as described by the manufacturer. DNA was eluted into 30 μl EB buffer (Qiagen).

4.6 Reamplification of Differential Display PCR Fragments

All PCR fragments recovered from the differential display gel could be reamplified with a set of universal primers, M13r(−48) primer [AGCGGATAACAATTTCACACAGGA] and T7 primer [GTAATACGACTCACTATAGGGC]. A 40 μl PCR was set up on ice with 3 μl template (see 4.5.), 1×PCR buffer, 1.5 mM MgCl₂, 20 μM dNTP, 0.2 μM T7 primer, 0.2 μM M13r(−48) primer and 2 U Taq polymerase (Qiagen) and run as described above (see 4.3.).

4.7 Electrophoresis on a Preparative 1.2% Agarose Gel

30 μl of reamplified PCR sample were mixed with 6 μl loading dye and separated on an 1.2% agarose/1×TBE gel together with a size standard and a PCR marker (Promega). Bands were cut out with a scalpel and DNA extracted from agarose gel slice using QIAquick gel extraction Kit as described (Qiagen). 1 μl of recovered DNA was used for sequencing.

5. Preparation of cDNA Libraries and Probe Synthesis

Since the availability of heart material is very limiting, labeled in vitro transcripts of a cDNA library prepared from heart mRNA were used for dot blot hybridization instead of reverse transcribed mRNA itself.

5.1 Preparation of a cDNA Library

5 μg of high quality mRNA (see 1., see 2.) were used to prepare a cDNA library using the cDNA Synthesis Kit and ZAP-cDNA Gigapack III Gold Cloning Kit (Stratagene) as described in the manual with the following modifications:

(a) Packaging and titering: 2.5 μl of the ligation reaction were packaged. If the library did not represent at least one million clones, the remaining 2.5 μl were also packaged. After centrifugation of XL1-Blue MRF′ culture (50 ml), the cells were gently resuspended in 10 mM MgSO₄ at 4° C. and immediately used for transduction or stored for max 40 h at 4° C.

(b) Determination of the insert size: 25 plaques were transferred from agar plates used for titering directly into 40 μl PCR premix (1×PCR-buffer, 0.25 μM T3 primer, 0.25 μM T7 primer, 200 μM dNTP, 0.085 U Taq DNA-polymerase) and inserts amplified using 35 cycles and an annealing temperature of 48° C. The insert size was checked on an agarose gel and was in the range of 1-2 kb.

(c) Storage of the library: Libraries were transferred into 50 ml-polypropylene tubes, supplemented with 150 μl 0.3% chloroform and stored at 4° C. A part of each library was stored in 7% DMSO at −80° C.

Mass in vivo-excision was done according to the protocol of the ZAP-cDNA Gigapack III Gold Cloning Kit with the following modifications:

Transfected XL1 Blue MRF′ were grown in 5 ml LB. 5 ml of the supernatant containing single stranded phages was used to infect 20 ml of SOLR cells. Remaining 20 ml of single stranded phages were stored at 4° C. for up to two months. To determine the titer of excised phagemids 10 μl, 1 μl and 0.1 μl of infected SOLR cells were plated on LB/Amp dishes. If the titer was lower than one million, 5 ml or more of the remaining supernatant was used again to infect fresh SOLR cells. Infected SOLR cells (25 ml) were grown in 200 ml LB/Amp over night for plasmid isolation (Plasmid Midi Kit, Qiagen).

5.2 Linearization of the template cDNA library for in vitro transcription

200 μg plasmid DNA were digested with Mol over night at 37° C. in a volume of 250 μl to linearize the plasmid at the 3′ end of the insert. The sample was controlled for complete digestion on an agarose gel, treated with 10 μg/μl Proteinase K (Roche) at 37° C. for 30 min, extracted once with TE saturated phenol (pH) 7.5-8) and once with chloroform/isoamytalcohol (24/1) and precipitated in the presence of 0.1 volume 3 M NaOAc (pH 5.2) and 3 volume EtOH. The pellet was washed with 500 μl 75% ethanol, dried at RT for 10 min, dissolved in 150 μl DEPC-treated water and quantified.

1 μg of linearized plasmid was used for an in vitro transcription as described (see 5.3.), omitting the radioactive labeled nucleotide and adding UTP to a final concentration of 10 mM. Following DNaseI digestion, the RNA was extracted with phenol/chloroform/isoamylalcohol (24/23/1), precipitated with EtOH and dissolved in 15 μl DEPC-treated water. The yield was in the range of 15-22 μg RNA. 1.5 μl RNA were separated on a formaldehde agarose gel. A smear of transcripts was visible between 0.5 kb and 10 kb with a peak at about 1 kb.

5.3 In Vitro Transcription

According to the RNA Transcription Kit (Stratagene) 1 μg of linearized template (see 5.2.) was incubated in the presence of 1× transcription buffer, 10 mM ATP, 10 mM CTP, 10 mM GTP, 1 mM UTP, 70 μCi [α-³³P]UTP (APB), 0.75 M DTT, 20 U rRNasin (Promega) and 25 U T3 RNA polymerase for 30 min at 37° C. After addition of 5 U RNase-free DNaseI (Roche) the sample was incubated for 15 min at 37° C. 25 μl STE-buffer (APB) was added to the probe and the reaction purified using G50 Micro Columns (APB) according to the manufacturers protocol.

5.4 Prehybridization of In Vitro Transcripts

To suppress probe hybridization to human repetitive DNA, labeled RNA was prehybridized to cot1-DNA. 213 μl DEPC-treated water, 100 μl 20×SSC, 2 μl 20% SDS and 40 μl cot1-DNA (1 μg/μl Gibco BRL) were added to 45 μl labeled RNA (see 5.3.), denatured at 95° C. for 2 min and incubated for 2 h at 65° C.

6 Quantitative Dot Blot Analysis

6.1 Transfer of PCR Fragments onto Nylon Membrane

For spotting, approximately 300 ng PCR product (see 3.2.) or gene-specific control cDNA fragments were mixed with 140 μl 0.4 M NaOH/10 mM EDTA pH 8.0 in 96 well microplates and denatured 10 min at 95° C. 50 μl of each PCR-fragment (at least 100 ng cDNA) were transferred on a nylon membrane (11.4×7.5 cm, BioRad) using a 384 hole vacuum apparatus (Keutz, custom-made). 50 μl 0.4 M NaOH were added to each position and transferred. The membrane was washed in 2×SSC, dried for at least 1 h at RT and fixed by UV crosslinking (Stratalinker 2400, Stratagene). For each experiment two identical membranes were prepared in parallel.

6.2 Dot Blot Hybridization and Washing

The cDNA filter was soaked in 2×SSC and transferred into a hybridization flask. The membrane was hybridized with 10 ml hybridization solution (6×SSC, 5× Denhardts, 0.2% SDS, 0.2% sodium pyrophosphate) supplemented with 50 μg/ml denatured salmon sperm DNA (Typ III, Sigma) at 65° C. for 2 h in an Unitherm 6/12 hybridization oven (UniEquip). The prehybridization mix was poured off. 200-400 μl of cot1-hybridized probe (see 5.4.) were added to 8 ml of hybridization solution (including salmon sperm DNA) preheated to 65° C. Dot blots were hybridized over night at 65° C. For washing of cDNA filters all solutions were heated to 65° C. The membrane was washed twice with 50 ml wash solution 1 (2×SSC, 0.1% SDS) for 30 min, then twice with 50 ml wash solution 2 (0.1×SSC, 0.1% SDS) for 30 min and wrapped in a keep-fresh foil. The filter was exposed to a phosphor screen for two days and scanned at 450 nm using the Storm Phosphoimager (Molecular Dynamics).

6.3 Data Analysis

Signal intensities were calculated using ImageQuant Software (Molecular Dynamics) by subtracting the local background. For comparison of different filters signal intensities were normalized by adjusting the overall intensity of each filter to 100%. In general, two cDNA filters were hybridized successively with 10 probes prepared from different human heart samples.

Dots which represented at least two fold changes in signal intensity comparing the group of DCM heart samples (y) with that of normal controls (x) were selected for further analysis. The probability of type 1 error was calculated to be less than 5% using the Wilcoxon test This non-parametric statistic algorithm does not assume any distribution of x and y values. If the sample size of one group was smaller than 4 the Wilcoxon test could not be applied. Instead significance of gene regulation was confirmed by a t-test. The t-test assumes that standard deviations of both groups x and y are similar and values distributed according to normal distribution.

Independent of the disease individual differences between human samples are expected. They are the result of the different genetic background of individuals, sex, age, environmental and life conditions (e.g. smoking, drinking, nourishment), the status of disease and medical treatment. Especially DCM patients were treated by a number of drugs prior to heart transplantation. We laid down that the regulation has to be consistent in at least two DCM patients and more or less homogenous in all but one non-failing patient. Selected clones were grown in 5 ml LB/Amp from glycerol stocks (see 3.2.). Plasmids were isolated using the Plasmid Mini Kit (Qiagen) and sequenced.

6.4 Stripping of Dot Blot Membranes

cDNA filters were transferred into boiling stripping solution (0.1×SSC, 0.5% SDS) and incubated for 1 h at RT. This procedure was repeated until no more radioactivity could be detected by a Geiger-Müller counter. The filter again was wrapped in keep-fresh foil and stored at RT.

7. Full-Length Cloning:

Full-length cloning was performed using RT-PCR with oligonucleotides priming to the 5′- and 3′-ends of the sequence encoding the open reading frame. PCR-fragments were then purified by agarose gel-electrophoresis followed by gel elution using the gel purification kit from Qiagen. PCR-fragments were finally cloned into p201-DONOR (Life Technologies) or pTOPO2.1 (Invitrogen).

The cloned cDNAs were verified by sequencing. In addition, in vitro translations were performed using the TNT Quick Coupled Transcription/Translation Systems (Promega) in order to verify the correct molecular weight of the proteins encoded by a given cDNA. The full-length clones were named according to their ID number provided with the suffix “-cds” (xxxxx-cds). The proteins were named according to their ID number provided with the suffix “-pep” (xxxxx-pep).

8. Yeast Two-Hybrid System

8.1 Two-Hybrid Screen Protocol (Golemis et al., 1994).

The yeast two-hybrid vectors are described in section below. Yeast strains used were EGY48LacZ-GFP (ura3::6*LexOp-lacZ, lys2::6*LexOpCYC1GFP, his3, trp1, 6*LexAOp-LEU2, matα) and EGY199UL (ura3::6*LexOp-lacZ, his3, trp1, 6*LexAOp-LEU2, mat a). Yeast was grown in YPD or selective minimal medium (Sherman 1986). Transformations were done using the high-efficiency method of Gietz et al., 1992. The bait plasmids were first introduced in the yeast strain EGY48LacZ-GFP resulting in the strain EGY48LacZ-GFP-bait. Self activation of the bait was checked by plating the yeast on minimal glucose medium with or without X-Gal (5-bromo-4-chloro-3-indolyl-β-D-galactopyranoside). In parallel protein expression was verified by western blot analysis using a polyclonal rabbit anti-LexA antiserum. A human heart cDNA library (pJG#19) cloned (EcoRI/XhoI) in the vector pJG4-5 was then introduced in the EGY48LacZ-GFP-bait strain. After transformation 4×10⁴ colonies per plate) yeast were plated on selective medium (−histidine, −tryptophane, +methionine, glucose). Colonies were harvested and an aliquot was plated on selective medium (−histidine, −tryptophane, −uracil, raffinose, galactose, X-gal). The interactions were assayed by colony growth on selective medium as well as by β-galactosidase activity on the plate. Positive clones were plated over night on medium (−histidine, −tryptophane, −uracil, glucose, X-gal) in order to deactivate the expression of the prey. The verification of the interaction was performed by plating the colonies on medium A:(−histidine, −tryptophane, −uracil, glucose, X-gal) and medium B: (−histidine, −tryptophane, −uracil, raffinose, galactose, X-gal). Only blue colonies growing on medium B but not on medium A were further analysed by yeast-colony-PCR. Plasmids were rescued and introduced in E. coli (Robzyk and Kassir, 1992). DNA was isolated from the bacteria and sequenced. Interactions were finally verified by reintroducing the plasmid (prey) in the yeast strain EGY199UL. Mating of the EGY199UL (prey) with the corresponding EGY48LacZ-GFP (bait) was performed in order to get a diploid strain carrying bait and prey (Guthrie and Fink, 1991; Pringle et al., 1997; Golemis and Khazak, 1997). Protein interaction resulted in growth and blue colour of the diploid colonies on medium B but not on medium A. Interactions were further analysed by quantifying the relative activity of the GFP reporter in a FACS assay.

8.2 Two Hybrid Vectors Description

8.2.1 Bait Vectors

1) pSH2-1 (Hanes S D. and Brent R. 1989)

2) pEG202(U8996)

3) 413MetLexN0

The vector 413MetLexN0 was constructed by cloning a PCR generated full length LexA repressor cDNA (with XbaI/BamHI overhangs) into the vector 413Met25 (Mumberg et al., 1994) cut XbaI/BamHI.

4) 413MetLexN0.att

The destination vector 413MetLexN0.att was constructed by introducing the rfC cassette of the Gateway™ system (Invitrogen) into the vector 413MetLexN0. For this purpose a linear PCR fragment comprising the rfC-cassette and flanking homologies of 40 bp to the LexA gene or 40 bp (5-prime) of the CYC1 terminator (3-prime) of the vector 413MetLexN0 was used for homologous recombination to the EcoRI linearized vector 413MetLexN0 in yeast. One correct recombinant vectors was re isolated from yeast and can be used for cloning of cDNAs by in vitro recombination performing a LR-reaction of the Gateway™ system.

5) 413MetLexC0

The vector 413MetLexC0 was constructed by cloning a PCR generated full length LexA repressor cDNA (with HindIII-ClaI-XhoI/SalI overhangs) into the vector 413Met25 (Mumberg D et al., 1994) cut HindIII/XhoI.

6) 413MetLexC0.att

The destination vector 413MetLexC0.att was constructed analogous to the procedure described for the vector 413MetLexCN.att.

8.2.2 Prey Vectors

1) pJG4-5(U89961)

2) 424 GBN0

The vector 424 GBN0 was constructed by cloning a PCR generated full length B42 transactivation domain cDNA (with XbaI/BamHI overhangs) derived from the vector pJG4-5 into the vector 424GAL1 (Mumberg D et al., 1994) cut SpeI/BamHI.

3) 424GBN0.att

The destination vector 424 GBN0.att was constructed by introducing the rfC cassette of the Gateway system (Invitrogen) into the vector 424 GBN0. For this purpose a linear PCR fragment comprising the rfC-cassette and flanking homologies of 40 bp to the LexA gene or 40 bp (5-prime) of the CYC1 terminator (3-prime) of the vector 424 GBN0 was used for homologous recombination to the EcoRI linearized vector 424GBN0 in yeast. One correct recombinant vector was re-isolated from yeast and can be used for cloning of cDNAs by in vitro recombination performing a LR-reaction of the Gateway™ system.

4) 424 GBC0

The vector 424 GBC0 was constructed by cloning a PCR generated full length B42 transactivation domain cDNA (with HindIII-ClaI-XhoI/SalI overhangs) into the vector 424GAL1 (Mumberg D et al., 1994) cut HindIII/XhoI.

5) 424 GBC0.att

The destination vector 424 GBC0.att was constructed analogous to the procedure described for the vector 424GBCN.att.

8.3 Two-Hybrid Interaction Matrix (40K Matrix)

A collection of yeast two-hybrid 200 plasmids (baits and preys) made at Medigene was introduced in EGY48LacZ-GFP and EGY199UL respectively. Each EGY48LacZ-GFP-bait were challenged against each EGY199UL-prey for interaction via mating (Golemis and Khazak, 1997). The resulting interactions tested were 40.10. This procedure correspond to the MediGene 40K matrix. Positive interaction were scored by growth on selective medium and β-galactosidase activity. Moreover, the strength of the interactions were quantified in a FACS assay. All interactions-were stored in the programme CACI (Computer analysis of Complex Interactions). Matrix interaction analysis was performed using the programme CACI.

9. Recombinant Gene Expression in Cardiomyocytes

9.1 Isolation of Primary Cardiomyocytes from Neonatal Rats

Neonatal rats (P2-P7) were sacrificed by cervical dislocation. The ventricles of the beating hearts were removed and cardiomyocytes were isolated with the “Neonatal Cardiomyocyte Isolation System” (Worthington Biochemicals Corporation, Lakewood, N.J.) according to the protocol. Briefly, the ventricles were washed twice with ice cold Hank's Balanced Salt Solution without Potassium and Magnesium (CMF-HBBS) and minced with a scalpel to an average volume of one cubic millimeter. The heart tissue was further digested over night with trypsin at 10° C. Next morning trypsin inhibitor and collagenase were added. After an incubation at 37° C. and mild agitation for 45 minutes the cells were dispersed by pipetting. The solution was further purified by 70 μm mesh (Cell Strainer) and centrifuged twice for 5 minutes at 60×g. The cell pellet was resuspended in plating medium and counted. Cells were seeded with a density of 2×10⁴/cm² on gelatine (Sigma, Deisenhofen) coated dishes. The next morning cells were washed twice with DMEM and maintenance medium was added.

| | |
|---|---|
| Plating medium: | DMEM/M-199 (4/1); 10% Horse serum, |
| | 5% Fetal calf serum; 1 mM sodiumpyruvate; |
| | antibiotics and antimycotics |
| Maintenance medium: | DMEM/M-199 (4/1); 1 mM sodiumpyruvate |

9.2 Construction of Expression Plasmids for Cardiomyocytes

The pCI-vector (Promega) was cut with BsrGI. The linearized vector was incubated with the Klenow-fragment and dNTPs to generate blunt ends. The resulting vector was cut with NheI and NotI after religation and gel purified. A PCR fragment comprising the entire open reading frame without the start codon of the yellow variant of the green fluorescent protein (YFP) was inserted into the NheI and NotI sites. The PCR was performed under standard conditions with the following primers to add several unique restriction site for further cloning:

5′-primer: SpeI-XbaI-EcoRI-XhoI-YFP

5′-GGA CTA GTT CTA GAG AAT TCC TCG AGG TGA GCA AGG
GCG AGG AG-3′

3′-primer: YFP-STOP-NotI (the NotI site was derived from the vector)

5′-AGT TGG TAA TGG TAG CGA CC-3′

template: pEYFP-vector (Clontech)

The PCR product was gel purified and digested with SpeI and NotI the generate compatible ends. The resulting vector was linearized with XbaI and EcoRI and gel purified in order to insert a consensus Kozak-sequence, which was derived from oligo annealing.

| | |
|---|---|
| 5′-Kozak: | 5′-CTA GAA CTA GTT CCA CCA TGG-3′ |
| 3′-Kozak | 5′-AAT TCC ATG GTG GAA CTA GTT-3′ |

In the final construction step the plasmid was linearized with EcoRI and XhoI and gel purified. A PCR fragment comprising the entire open reading frame of 66268 flanked by an EcoRI site at the 5′-end and a XhoI site at the 3′-end was inserted.

9.3 Stimulation of Isolated Cardiomyocytes from Neonatal Rats

Stimulation of primary cardiomyocytes from neonatal rats (pCMs) was started two to six hours after medium was changed to maintenance medium. Directly after stimulation pCMs were infected with recombinant adenoviruses at a MOI of five. Cells were incubated for 48 hours at humidified atmosphere at 37° C. and 5% CO₂ followed by an analysis of morphological alterations.

9.4 Transient Transfection of Isolated Cardiomyocytes from Neonatal Rats

For each well of a six well plate 1 μg of plasmid DNA was combined with 20 μl 2×BBS and 100 μl maintenance medium without antibiotics. Meanwhile 4 μl of LIPOFECTAMINE (Gibco/BRL) were mixed with 650 μl maintenance medium without antibiotics in a polystyrene tube. The DNA-sample was added after an incubation for 15′ at room-temperature. The suspension was mix by inverting the tube twice and incubated for 15′ at room-temperature. Meanwhile medium was changed to 1 ml maintenance medium without antibiotics. The transfection-mixture was added onto the cells and gene expression was analysed 48 hrs later.

| | | |
|---|---|---|
| 2 × BBS: | 50 mM | BES |
| | 280 mM | NaCl |
| | 1.5 mM | Na₂HPO₄ |

adjust to pH 6.95 by administration of NaOH

### Example 2

EST 40399 (FIG. 1A) was identified by suppression subtractive hybridization comparing transcript levels of heart tissue explanted from normal control h92 with one from DCM patient h97 (see TABLE 1). The fragment was found to be over-represented in the DCM tissue.

As of FIG. 1D the identified cDNA fragment is a part of the EST clone NM_{—}003970(FIG. 1B), which encodes the amino acid sequence NP_{—}003961 (identical to CAA48832; FIG. 1C). This amino acid sequence encodes the 165 kDa M-protein, also known as myomesin 2 or MYOM2.

Z and M bands of the sarcomere are interconnected by the long titin molecules. The 165 kDa M-protein is one of two known titin-associated proteins, which seem responsible for the formation of a head structure on one end of the 0.9 micron long titin string (Vinkemeier et al.). M-protein may function in strengthening the links between thick filaments necessary to withstand the stronger tension during contraction in the heart and in fast fibers (van der Ven et al.)

Upregulation upon DCM was confirmed for two additional DCM patients compared to five normal control hearts by quantitative dot blot analysis (FIG. 1E). The relative expression level of 40399 is induced by a factor of 3.1 upon disease. The probability of type 1 error is less than 5% as determined in a t-test.

Expression was not induced in two DCM patients, which may reflect individual differences throughout the population.

Significant upregulation of 40399 expression in heart tissue of two DCM patients compared to five normal controls indicates that an increased expression of 40399 is associated with dilated cardiomyopathy. Upregulation of titin-associated muscle M-protein by a factor of 3 may massively interfere with normal myofibril assembly and stabilization and decrease muscular activity. From our data we conclude that abnormalities in expression of this protein are associated with muscular abnormalities that result in cardiomyopathies. Therefore we expect the protein to play a causative role in heart diseases, especially congestive heart failure.

Mutations in other sarcomeric proteins have already been identified as causes of hypertrophic cardiomyopathy, suggesting that cytoskeletal proteins play a central role in cardiac function (Hein et al.). These findings support our general observation of a causative correlation between deregulation of sarcomeric proteins and reduced contractile function in end-stage heart failure. Therefore, 40399 can serve as a heart disease marker and a specific molecular target for drug development.

Downregulation of protein expression by specific inhibitors or antisense constructs seems to be a very promising therapeutic tool to treat heart diseases.

### Example 3

EST 41441 (FIG. 2A) was identified by suppression subtractive hybridization comparing transcript levels of heart tissue explanted from normal control h92 with one from DCM patient h97 (see TABLE 1). The fragment was found to be over-represented in the control tissue. The identified cDNA fragment is a part of the EST clone AW755252 (FIG. 2B), which predicts an amino acid sequence 41441pep given in FIG. 2C (schematic alignment FIG. 2D).

Downregulation upon DCM was confirmed for four DCM patients compared to five normal control hearts by quantitative dot blot analysis. The relative expression level of 41441 is reduced by a factor of 4.5 upon disease (FIG. 2E). The probability of type 1 error is less than 5% as determined in a Wilcoxon test.

The EST clone AW755252 (Walker et al.) was isolated from a human cardiac muscle expression library and found to be similar to cardiomyopathy associated gene 3 (CMYA3, unpublished).

The LIM sequence motif is a part of the cardiomyopathy associated gene 3.

The LIM sequence motif was first identified in homeodomain proteins Lin-11, Is1-1 and Mec-3. The LIM domain is a double zinc finger motif that mediates the protein-protein interactions of transcription factors, signaling- and cytoskeleton-associated proteins. There is no evidence, that LIM domains bind DNA directly. Instead, an increasing number of studies implicate LIM domains in protein-protein interactions that regulate development, cellular differentiation and the cytoskeleton (Bach).

Yeast Two-Hybrid Interactions

Interactors with the protein coded by 41441pep were screened using 41441pep as a bait. A large screen was performed using 4 large plates for the library transformation which led to the analysis of 2×10⁷ clones. The two hybrid procedure described (protocol 22) led to the identification of 4 different interacting partners. The corresponding cDNAs were identified by homology search using the first 500 nucleotides sequence of the pray clone. The partners are: Hepatitis B virus interacting protein (AF029890), U6 snRNA-associated Sm-like protein LSm8 (AF182294), unknown protein HSPC297 (AF161415) and supervillin (AF051851).

Hepatitis B Virus Interacting Protein or XIP

The identity with Hepatitis B virus interacting protein (AF029890) was found to be 100% over the first 400 amino acids. The homology starts at nucleotide 9 of the AF029890 sequence. The XIP cDNA recognizes a single 0.7 kb transcript in all tissues studied and was particularly abundant in skeletal and cardiac muscles tissues (Melegari et al., 1998). The XIP protein was also found to interact with the hepatitis B virus protein HBx (Melegari et al., 1998). Interestingly, over-expression of the XIP protein prevented wild-type HBx activity on such promoters as well as reduced HBV replication to levels comparable to those observed with an HBx-minus variant strain (Klein et al., 1999.)

U6 snRNA-Associated Sm-Like Protein LSm8

The sequence revealed 100% homology to Homo sapiens U6 snRNA-associated Sm-like protein LSm8 over 400 nucleotides. The homology starts at nucleotide 31 of the AF 182294 sequence. The yeast homologue of Lsm8 seems to be play a role, together with Lhp1, as a molecular chaperone of polymerase III. Lsm8 might be implicated in the very early steps of the U6 snRNP assembly (Panome et al., 1998).

Supervillin

Homology search using the interactor of clone 41441 led to the identification of supervillin (SVIL) (XM_{—}011894, AF109135) with 99% identity. Supervillin RNA are expressed ubiquitiniously. The human supervillin gene is localized to a single chromosomal locus at 10p11.2 a region that is deleted in some prostate tumours as well as in so tumour cell lines (Pope et al., 1998). The cDNA sequence of this interactor showed identity to supervillin isoform 2, a membrane associated F-actin binding protein. This protein is also known as archvillin or p205. The identity starts at amino acid 1872 and stops at 1997. Alignment with clones of the database showed that the bait encodes the C-terminal part of the protein supervillin. In this sequence the motif GEL (Gelsolin homology domain) could be identified from amino acid 39 to 138. This domain was also found in Gelsolin/severin/villin. It is thought to exist both as a intra- and extracellular domain and and may be responsible for Calcium-binding as well as actin-binding. This protein is tightly associated with both actin filaments and plasma membrane specifically in focal adhesion plaques. Over-expression of full-length supervillin in these cells disrupts the integrity of focal adhesion plaques and results in increased levels of F-actin and vinculin. Moreover, supervillin contains nuclear targeting signals in the centre of the protein which seem to be functional. Therefore supervillin may contribute to cytoarchitecture in the nucleus as well as he plasma membrane (Wulfkuhle et al., 1999).

Significant downregulation of 41441 expression in heart tissue of four DCM patients compared to five normal controls indicates that a lowered expression of 41441 is associated with dilated cardiomyopathy. Lowered expression of 41441 by a factor of 4-5 seems to induce a cardiomyopathic phenotype. Therefore we expect the protein to play a causative role in heart diseases, especially congestive heart failure.

The predicted functional domain LIM_{—}1 also indicates a major role of 41441 in regulation of development, cellular differentiation or the cytoskeleton. From our data together with those from Genbank entree AW755252 we conclude that 41441 is predominantly expressed in cardiac muscle, which supports our idea that 41441 can serve as a marker for heart diseases and a specific molecular target for drug development.

Upregulation of protein expression by gene therapeutic intervention, compensatory molecules or specific activators seems to be a very promising therapeutic tool to treat heart diseases.

### Example 4

EST 52706 (FIG. 3A) was identified by suppression subtractive hybridization comparing transcript levels of heart tissue explanted from normal control KN2 with one from DCM patient DHZM3 (see TABLE 1). The fragment was found to be over-represented in the diseased tissue.

EST 52706 (FIG. 3A) was found to be repressed upon disease in screens for expression profiles using suppression subtractive hybridization (?). Transcript levels are significantly downregulated by a factor 27.3 in five DCM patients compared to five normal controls (FIG. 3B). The probability of type 1 error is less than 5% as determined in a Wilcoxon test. Significant homologies to known sequences from Genbank were not found.

Significant downregulation of 52706 expression in heart tissue of six DCM patients compared to the same number of normal controls indicates that a lowered expression of 52706 is associated with dilated cardiomyopathy. The extreme decrease in expression of 52706 by a factor of 27 seems to induce a cardiomyopathic phenotype. Therefore we expect the protein to play a causative role in heart diseases, especially congestive heart failure. As a conclusion 52706 can serve as a marker for heart diseases and a specific molecular target for drug development.

Upregulation of protein expression by gene therapeutic intervention, compensatory molecules or specific activators may be a therapeutic tool to treat heart diseases.

### Example 5

EST 56461 (FIG. 4A) was identified by suppression subtractive hybridization comparing transcript levels of heart tissue explanted from normal control KN5 with one from DCM patient h52 (see TABLE. 1). The fragment was found to be over-represented in the DCM tissue.

The identified cDNA fragment was found to be overlapping with the EST clone AF077035 (FIG. 4B), which encodes the amino acid sequence AAD27768 (FIG. 4D). The predicted amino acid sequence for 56461 is shown in sequence 56461pep (FIG. 4C).

AF077035 was isolated from CD34(+) hematopoietic stem and progenitor cells (HSPC, Zhou et al.). The amino acid sequence of AAD27768 is to 91% identical to one translated from EST AW785791, which was identified to be specifically expressed in pooled tissues from Sus scrofa embryos (Fahrenkrug et al.).

Upregulation upon DCM was confirmed for two additional DCM patients compared to five normal control hearts by quantitative dot blot analysis (FIG. 4E). For these samples, DCM15 and DCM13, the relative expression level of 56461 is induced by a factor of 5.4. The probability of type 1 error is less than 1% as determined in a t-test.

The remaining three DCM patients did not show a significant change in 56461 expression, which may be the result of individual differences throughout the population.

Significant upregulation of 56461 expression in heart tissue of three DCM patients compared to six normal controls indicates that an increased expression of 56461 is associated with dilated cardiomyopathy. Increased expression of 56461 by a factor of 5-6 seems to induce a cardiomyopathic phenotype. Therefore we expect the protein to play a causative role in heart diseases, especially congestive heart failure.

Moreover, the homology to RNA binding domains may indicate a regulatory function for 56461. This finding supports our idea that 56461 can serve as a marker for heart diseases, especially congestive heart failure and a specific molecular target for drug development. Downregulation of protein expression by specific inhibitors or antisense constructs seems to be a very promising therapeutic tool to treat heart diseases.

### Example 6

EST 61105 (FIG. 5A) was identified by suppression subtractive hybridization comparing transcript levels of heart tissue explanted from normal control KN4 with one from DCM patient h94 (see TABLE 1). The fragment was over-represented in the control tissue. The identified cDNA fragment was found to be a part of the EST clone M14780 (FIG. 5B), which encodes the amino acid sequence AAA52025 (FIG. 5C; schematic alignment FIG. 5 D). This amino acid sequence encodes the muscle isoform of creatine kinase (creatine kinase M, Perryman et al.), which is one of the important structural and energy metabolism components in skeletal muscle. It catalyzes the reversible transfer of phosphoryl group from creatine phosphate to ADP to form ATP to sustain contractile activity.

Downregulation upon DCM was confirmed for five DCM patients compared to the same number of normal control hearts by quantitative dot blot analysis (FIG. 5E). The relative expression level of 61105 is significantly reduced by a factor of 4 upon disease. The probabilty of type 1 error is less than 5% as determined in a Wilcoxon test

Yeast Two-Hybrid Interaction

The interactors were identified using the 40K matrix of MediGene and analysed by MediGene CACI programme. The following three proteins interact with AAA52025: CapZa (P52907), c-Raf (P04049), FBP (AF049528).

CapZa

CapZ alpha has been localized on Chromosome 1 at position 1p36.13-q23.3. CapZa is an Actin capping protein which bind as heterodimer F-actin at the fast growing end in a Ca2+ independent manner.

FBP11 (Formin Binding Protein):

Synonyms for FBP are: HYPA, huntingtin-interacting protein (AF049528, AF049524, AF049523) and Fas-ligand associated factor (U70667). FBP11-contains WW motifs that recognize PPXY or PPLP motifs to mediate the interaction (Bedford et al., 1997). Creatine-kinase-M contains a PPXY motif at position 143.

c-Raf (Isoforme of Raf-1)

c-Raf was localised on chromosome 3 a locus 3p25. This protein belongs to the Ser/Thr family of protein kinase, it contains a zinc-dependent phorpbol-ester and DAG binding domain. Moreover, a relationship between c-Raf and Creatine kinase has been shown by other groups in myoblasts (Coolican et al., 1997; Samuel, 1999) and in rhabdomyosarcoma (Ramp et al., 1992).

Significant downregulation of 61105 expression in heart tissue of five DCM patients compared to the same number of normal controls indicates that a lowered expression of 61105 is associated with dilated cardiomyopathy. Downregulation of creatine kinase M by a factor of 4 massively decreases the energy reservoir which is necessary to sustain muscle contractility. Therefore we expect the protein to play a causative role in heart diseases, especially congestive heart failure.

The protein expression was also observed to be deregulated upon canine rapid ventricular pacing, which produces a low output cardiomyopathic state similar to DCM (Heinke et al.). Taken together, these results strongly support the notion that energy production is impaired and mitochondrial dysfunction is involved in the development of heart failure. These findings support our general observation of a causative correlation between energy depletion and end-stage heart failure. Therefore, 61105 is a marker and in our opinion also a specific molecular target for drug development.

Upregulation of protein expression by gene therapeutic intervention, compensatory molecules or specific activators seems to be a very promising therapeutic tool to treat heart diseases. In general, increasing the level of available energy sources for muscle contraction by increasing the concentration of free ATP or creatine phosphate would be of great benefit in treating heart failure.

### Example 7

EST 61166 (FIG. 6A) was identified by suppression subtractive hybridization comparing transcript levels of heart tissue explanted from normal control KN4 with one from DCM patient h94 (see TABLE 1). The fragment was over-represented in the control tissue.

Using LabOnWeb (Compugen) it was possible to assemble 61166contig (FIG. 6B) that codes for a predicted protein with the amino acid sequence of 61166pep (FIG. 6C). The assembly of EST is shown in FIG. 6D with examples of known ESTs (AI 745235,AL 050107, AI 927050)

61166 displays a significant homology to human 65 kDa yes-associated protein YAP65 (NM_{—}006106, Expect=2e-84, Identity 57%, Wambutt et al.). YAP65 associates in vitro with the Src homology domain 3 (SH3) of the Yes proto-oncogene product (yes kinase) and other signaling molecules (Sudol et al.). The motif PVKQPPPLAP of human YAP65, which binds to SH3 domains is not conserved in 61166 (amino acids 201-210 marked in italic letters above).

Downregulation upon DCM was confirmed for five DCM patients compared to the same number of normal control hearts by quantitative dot blot analysis (FIG. 6E). The relative expression level of 61166 is significantly reduced by a factor of 3.9 upon disease. The probability of type 1 error is less than 5% as determined in a Wilcoxon test.

Significant downregulation of 61166 expression in heart tissue of five DCM patients compared to five normal controls indicates that a lowered expression of 61166 is associated with dilated cardiomyopathy. Lowered expression of 61166 by a factor of 4 seems to induce a cardiomyopathic phenotype. Therefore we expect the protein to play a causative role in heart diseases, especially congestive heart failure.

The high homology to a yes kinase associating protein suggests a central role for 61166 in signal transduction or development. This finding supports our idea that 61166 can be used as a specific molecular target for drug development and/or diagnostics.

Upregulation of protein expression by gene therapeutic intervention, compensatory molecules or specific activators may be a therapeutic tool to treat heart diseases.

### Example 8

Screen for expression profiles using a dot blot ybridization in a higher number of patients clearly showed that 61244 is induced upon disease (FIG. 7E). Transcript levels are significantly upregulated by a factor 3.6 in five DCM patients compared to five normal controls. The probability of type 1 error is less than 5% as determined in a Wilcoxon test.

EST 61244 (FIG. 7A) was identified by suppression subtractive hybridization comparing transcript levels of heart tissue explanted from normal control KN4 with one from DCM patient h94 (see TABLE 1). The fragment was found to be over-represented in the control tissue. The identified cDNA fragment was found to be a part of the EST clone AF161698 (FIG. 7B), which encodes the amino acid sequence AAD45360 (FIG. 7C). This amino acid sequence encodes the Apolipoprotein B mRNA editing protein 2 (APOBEC-2). An overview of the mentioned sequences is depicted in FIG. 7D.

(APOBEC-2) is highly similar and evolutionarily related to APOBEC-1, which mediates the editing of apolipoprotein (apo) B mRNA (Liao et al.). Both proteins are members of C (cytidine)-->U (uridine) editing enzyme subfamily of the cytidine deaminase supergene family.

APOBEC-2 does not display detectable apoB mRNA editing activity. Like other editing enzymes of the cytidine deaminase superfamily, APOBEC-2 has low, but definite, intrinsic cytidine deaminase activity. APOBEC-2 mRNA and protein are expressed exclusively in heart and skeletal muscle.

Yeast Two-Hybrid Interaction

The interaction of AAD45360 (APOBEC-2) was analysed by challenging this bait (against 4×10⁴ clones). The two-hybrid analysis procedure led to the identification of one interacting partner. This partner was identified by homology search using the first 500 nucleotides sequence of the pray clone. This partner is beta myosin heavy chain (M21665).

The prey cDNA showed 99% homology with beta myosin heavy chain (M21665). Kurabayashi et al., (1988) showed that the beta myosin heavy chain expression is predominantly expressed in the ventricle. Furthermore, the authors show that beta-form MHC mRNA is expressed in adult atrium at a low level but scarcely expressed in fetal atrium. Moreover, mutation of the beta myosin heavy chain have been reported to play a role in heart hypertrophy (Enjuto et al., 2000; Greber-Platzer et al., 2001).

Significant upregulation of 61244 expression in heart tissue of five DCM patients compared to five normal controls indicates that an increased expression of 61244 is associated with dilated cardiomyopathy. Increased expression of 61244 by a factor of 3-4 seems to induce a cardiomyopathic phenotype. Therefore we expect the protein to play a causative role in heart diseases, especially congestive heart failure.

Moreover, the protein is described to be specifically expressed in heart and skeletal muscle. Thus, 61244 may be a novel RNA editing enzyme with natural substrates in these tissues, that plays an important role in RNA modification. This finding supports our idea that 61244 is a specific molecular target for drug development and/or diagnostics.

Downregulation of protein expression by specific inhibitors or antisense constructs seems to be a very promising therapeutic tool to treat heart diseases.

### Example 9

Screen for expression profiles in a higher number of patients clearly showed that 65330 is induced upon disease (FIG. 8E). Transcript levels are significantly upregulated by a factor 2.2 in five DCM patients and 1.8 in two ICM patients compared to five normal controls. The probability of type 1 error is less than 5% as determined in a Wilcoxon test and t-test.

EST 65330 (FIG. 8A) was identified by suppression subtractive hybridization comparing transcript levels of heart tissue explanted from normal control KN6 with one from DCM patient h100 (see TABLE 1).

The identified EST was found to be a part of the EST clone AF249873 (FIG. 8D), which is itself a part of a 65330contig of assembled EST sequences (FIG. 8B). The EST clone AF249873 encodes the amino acid sequence AAF63623 (FIG. 8C). AF249873 encodes a novel gene located on human chromosome 4q with specific expression in cardiac and skeletal muscle (Ahmad et al.).

Yeast Two-Hybrid Interaction

4×10⁴ clones were challenged against the bait AAF 63623 (SMP). The all two-hybrid analysis, procedure led to the identification of one interacting partner: α-actinin 2 (M86406). This interactor was identified by homology search using the first 500 nucleotides sequence of the prey clone.

α-actinin 2

Homology search with sequences in the database showed 100% identity with α-actinin 2 (ACTN2) (NM_{—}001103). The homology starts at nucleotide 1469 of α-actinin 2. α-actinin 2 was mapped on chromosome 1q42-q43 and was found to be expressed in skeletal muscle as well as in heart muscle (Beggs et al., 1992).

Significant upregulation of 65330 expression in heart tissue of five DCM patients and two ICM patients compared to five normal controls indicates that an increased expression of 65330 is associated with dilated cardiomyopathy. According to its interaction with α-actinin, this protein might play a role in the cytoskeleton of a muscle cell. Therefore we expect the protein to play a causative role in heart diseases, especially in congestive heart failure.

Moreover, the protein is described to be specifically expressed in heart and skeletal muscle. This finding supports our idea that 65330 is a specific molecular target for drug development or diagnostics. Downregulation of protein expression by specific inhibitors or antisense constructs seems to be a very promising therapeutic tool to treat heart diseases.

### Example 10

EST 66214 (FIG. 9A) was identified by suppression subtractive hybridization comparing transcript levels of heart tissue explanted from a normal control (KN6) with one from a DCM patient (h100, see TABLE 1). The fragment was found to be over-represented in the DCM tissue.

The identified cDNA fragment is a part of the EST clone AF129505; the sequence of the 66214cds is shown in FIG. 9B.

AF129505 was described to be a novel X-chromosomal human gene (SMPX) encoding the amino acid sequence AAF19343 (9 D) which is a small muscular protein (Patzak et al.). The gene consists of five exons and four introns comprising together 52.1 kb and is preferentially and abundantly expressed in heart and skeletal muscle. The gene maps close to DXS7101 31.9 cM from the short arm telomere of the X-chromosome at Xp22.1. FIG. 9 C shows the amino acid sequence of 66214pep.

Upregulation upon DCM was confirmed for five DCM patients compared to four normal control hearts by quantitative dot blot analysis (FIG. 9E). The relative expression level of 66214 is significantly induced by a factor of 4.2 upon disease. The probability of type 1 error is less than 5% as determined in a Wilcoxon test.

The elevated expression observed for healthy patient h92 may represent individual differences throughout the population.

Yeast Two-Hybrid Interaction

The 4×10⁴ clones were analysed for the screen with 66214pep. The two-hybrid analysis procedure led to the identification of 3 different interactors: Daxx (AB015051), Rad6 (U38785), Ubc9 (P50550). These partners were identified by homology search using the first 500 nucleotides sequence of the pray clone.

Daxx

Search in the data base showed 99% identity with Daxx (AB015051) over the 400 nucleotides. The homology started at nucleotide 1936 of the Daxx sequence. Daxx was mapped on chromosome 6p21.3 (Kiriakidou et al., 1997). The identity found at nucleotide level was confirmed at amino acid level. Daxx was initially found as an interactor of Fas. (Yang et al. 1997). Like Fas, it is believed to activate the INK signal transduction cascade. Therefore, Daxx might play a role in apoptosis regulation.

Ubc9

The prey showed 100% identity with the human Ubc9 sequence the clone covered the all Ubc9 sequence. Ubc9 is thought to be involved in the ubiquitin-dependent protein degradation system (Wang et al. 1996). A single copy of the hUBC9 gene was found and localised to human chromosome 16p13.3. Interestingly the interaction of Daxx (see above) was already found with the Ubc9 protein (Ryu et al., 2000).

Rad6

Homology search led to the identification of RAD6 (U38785). This result was confirmed by the amino acid analysis. The involvement of RAD6 in the degradation of endogenous inducible cAMP early repressor (ICER) protein in primary cardiomyocytes and myogenic cell lines has been reported (Folco and Koren, 1997). Moreover, recent data showed that Ubiquitin-Conjugating Enzymes (rad6) Target Repressors of Cyclic AMP-induced Transcription for Proteolysis (Pati et al., 1999)

Significant upregulation of 66214 expression in heart tissue of six DCM patients compared to five normal controls indicates that an increased expression of 66214 is associated with dilated cardiomyopathy. Therefore we expect the protein to play a causative role in heart diseases, especially congestive heart failure.

Moreover, the protein is described to be preferentially and abundantly expressed in heart and skeletal muscle. This finding supports our idea that 66214 is a specific molecular target for drug development and/or diagnostics. Downregulation of protein expression by specific inhibitors or antisense constructs seems to be a very promising therapeutic tool to treat heart diseases.

### Example 11

66268 and 52474 (FIG. 10A) were identified by suppression subtractive hybridization comparing transcript levels of heart tissue explanted from normal control KN6 with DCM patient h100, and KN2 with DHZM3 (see TABLE 1), respectively. Both fragments were found to be over-represented in the DCM tissue. Both identified fragments are parts of the EST clone X83703 (FIG. 10B), which encodes the amino acid sequence CAA58676 (FIG. 10 C).

CAA58676 has been identified as a novel cytokine-inducible nuclear protein from human endothelial cells (C-193 or CARP, Chu et al.). C-193 represents a new member of the primary response gene family, since its mRNA expression is induced by IL1α, TNFα, LPS and CHX.

Dot blot hybridizations showed a slight increase in mean expression intensities of DCM patients versus normal controls for both fragments, but the variability from patient to patient was high and the dot blot result statistically was not significant applying a Wilcoxon or t-test. FIG. 10E depicts the example of the hybridization with clone 66268.

An overlapping fragment S1MC01-1 was identified to be induced upon DCM by means of differential display (FDD, see 4.). The differential display expression profile independently confirms upregulation of this gene by a factor of 2.2 upon DCM and [CM and 3.3 upon HCM. The probability of type 1 error for upregulation upon DCM is less than 5% as determined in a t-test.

Recombinant Over Expression in Primary Cardiomyocytes from Neonatal Rats:

A CAA58676-YFP fusion protein was over expressed in primary cardiomyocytes from neonatal rats (pCMs). The pCMs were stimulated with Phenylephrine (PE) which leads to flat cells with an extensive parallel sarcomer organization as could be detected in the upper left and lower right corner of FIG. 3. The cell over-expressing CAA58676 was detected by the fluorescence signal of the CAA58676-YFP fusion protein. The protein accumulated in litte aggregates in the nucleus. In addition, a thin, elongated shape of the cell was detectable, which pointed to the induction of a serial sarcomere organization after over expression of CAA58676. This observation augmented our opinion, that the over-expression of CAA58676 in the human failing heart has a causative role in disease establishment and progression, because the elongated shape of cardiomyocytes in combination with the serial sarcomere organization is a well known characteristic of diseased cells in the insufficient human heart.

Upregulation of 66268 and 52474 expression in heart tissue of DCM, ICM and HCM patients compared to normal controls indicates that an increased expression of 66268 and 52474 is associated with dilated, ischemic and hypertrophic cardiomyopathy. Increased expression of 66268 and 52474 by a factor of 2-3 seems to induce a cardiomyopathic phenotype. This was strongly supported by our functional analysis in pCMs. A recombinant over expression of a CAA58676-YFP fusion protein led to a serial sarcomere organization which is the main morphological characteristic of diseased cells in the failing human heart. Therefore we expect the protein to play a causative role in cardiomyopathies.

Moreover, the induction by cytokines as well as its mRNA and protein instability elements indicate an important regulatory function for 66268 and 52474 in signal transduction and control of secondary gene expression. Its ankyrin-like repeats may be involved in protein-protein interactions. These findings support our idea to use 66268 and 52474 as a specific molecular target for drug development and/or diagnostics.

Downregulation of protein expression by specific inhibitors or antisense constructs seems to be a very promising therapeutic tool to treat heart diseases.

1. A method for identifying a subject at risk for a disease of the heart, comprising the step of quantitating the amount of at least one RNA encoding an amino acid sequence selected from the group consisting of:
(a) the amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) an amino acid sequence that is at least 60%, preferably at least 80%, especially at least 90%, advantageously at least 99% identical to the amino acid sequence of (a);
(c) the amino acid sequence of (a) with at least one conservative amino acid substitution;
(d) an amino acid sequence that is an isoform of the amino acid sequence of any of (a) to (c);
(e) the RNA transcribed from the DNA sequence of SEQ ID NO: 10 [NM_{—}003970], the DNA sequence of SEQ ID NO: 11 [AW755252], the DNA sequence of SEQ ID NO: 12 [EST clone 52706], the DNA sequence of SEQ ID NO: 13 [EST clone 56461], the DNA sequence of SEQ ID NO: 14 [M14780], the DNA sequence of SEQ ID NO: 15 [61166contig], the DNA sequence of SEQ ID NO: 16 [AF161698], the DNA sequence of SEQ ID NO: 17 [65330contig], the DNA sequence of SEQ ID NO: 18 [66214cds] or the DNA sequence AF129505, or the DNA sequence of SEQ ID NO: 19 [X83703] or a degenerate variant thereof; and
(f) an amino acid that is encoded by a DNA molecule the complementary strand of which hybridizes in 4×SSC, 0.1% SDS at 65° C. to the DNA molecule encoding the amino acid sequence of (a), (c) or (d), in the heart tissue of the subject.

2. The method according to claim 1, wherein the amount of the said RNA is quantitated using a nucleic acid probe which is a nucleic acid comprising a sequence selected from the group consisting of:
(a) the DNA sequence of the RNA transcribed from the DNA sequence of SEQ ID NO: 10 [NM_{—}003970], the DNA sequence of SEQ ID NO: 11 [AW755252], the DNA sequence of SEQ ID NO: 12 [EST clone 52706], the DNA sequence of SEQ ID NO: 13 [EST clone 56461), the DNA sequence of SEQ ID NO: 14 [M14780], the DNA sequence of SEQ ID NO: 15 [61166contig], the DNA sequence of SEQ ID NO: 16 [AF161698], the DNA sequence of SEQ. ID NO: 17 [65330contig], the DNA sequence of SEQ ID NO: 18 [66214cds] or the DNA sequence AF129505, or the DNA sequence of SEQ ID NO: 19 [X83703] or a degenerate variant thereof;
(b) a DNA sequence at least 60%, preferably at least 80%, especially at least 90%, advantageously at least 99% identical to the DNA sequence of (a);
(c) a nucleic acid sequence that encodes the amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; each of said amino acid sequences having at least one conservative amino acid substitution;
(d) a nucleic acid sequence that encodes an amino acid sequence that is at least 60%, preferably at least 80%, especially at least 90%, advantageously at least 99% identical to the amino acid sequence of (c);
(e) a nucleic acid sequence that encodes the amino acid sequence of (c) or (d) with at least one conservative amino acid substitution;
(f) a nucleic acid sequence that hybridizes in 4×SSC, 0.1% SDS at 65° C. to the complementary strand of the DNA molecule encoding the amino acid sequence of (c), (d) or (e); and
(g) a fragment of at least 15 nucleotides in length of (a) to (f), wherein the nucleic acid is detectably labeled; or
(h) a nucleic acid probe comprising a sequence that specifically hybridizes under physiological conditions to the nucleotide sequence selected from the group consisting of:
(i) the DNA sequence of the RNA transcribed from the DNA sequence of SEQ ID NO: 10 [NM_{—}003970], the DNA sequence of SEQ ID NO: 11 [AW755252], the DNA sequence of SEQ ID NO: 12 [EST clone 52706], the DNA sequence of SEQ ID NO: 13 [EST clone 56461], the DNA sequence of SEQ ID NO: 14 [M14780], the DNA sequence of SEQ ID NO: 15 [61166contig], the DNA sequence of SEQ ID NO: 16 [AF161698], the DNA sequence of SEQ ID NO: 17 [65330contig], the DNA sequence of SEQ ID NO: 18 [66214cds] or the DNA sequence AF129505, or the DNA sequence of SEQ ID NO: 19 [X83703]
(ii) a DNA sequence at least 60%, preferably at least 80%, especially at least 90%, advantageously at least 99% identical to the DNA sequence of (i);
(iii) a nucleic acid sequence that encodes the amino acid sequence SEQ ID NO: 1 [NP_{—}00396], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino sequence of SEQ ID NO: 9 [CAA58676] with at least one conservative amino acid substituion;
(iv) a nucleic acid sequence that encodes an amino acid sequence that is at least 60%, preferably at least 80%, especially at least 90%, advantageously at least 99% identical to the amino acid sequence of (iii);
(v) a nucleic acid sequence that encodes the amino acid sequence of (iii) with at least one conservative amino acid substitution; and
(vi) a nucleic acid sequence that hybridizes in 2×SSC, 0.1% SDS at 65° C. to the DNA molecule encoding the amino acid sequence of (iii), (iv) or (v),
(vii) a fragment of at least 15 nucleotides in length of (i) to (vi).

3. A method for identifying a subject at risk for a disease of the heart, comprising the step of quantitating the amount of a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
in the heart tissue of the subject.

4. The method according to claim 3, wherein the amount of the said polypeptide is quantitated using an antibody or an antigen-binding portion of said antibody that specifically binds a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution.

5. The method according to claim 4, wherein said antibody or antibody binding portion is or is derived from a human antibody or a humanized antibody.

6. The method according to claim 4 or claim 5, wherein the antibody, the binding portion or derivative thereof is detectably labeled.

7. The method of claim 6, wherein said derivative of said antibody is an scFv fragment.

8. The method of claim 1 or 2, wherein said RNA is obtained from heart tissue.

9. The method of any one of claims 3 to 7 wherein said polypeptide is quantitated in heart tissue.

10. The method of any one of claims 1, 2 and 8 further comprising the step of normalizing the amount of RNA against a corresponding RNA from a healthy subject or cells derived from a healthy subject.

11. The method of any one of claims 3 to 7 and 9 further comprising the step of normalizing the amount of polypeptide against a corresponding polypeptide from a healthy subject or cells derived from a healthy subject.

12. A method for identifying a compound that increases or decreases the level in heart tissue of a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
said method comprising the steps of:
(1) contacting a DNA encoding said polypeptide under conditions that would permit the translation of said polypeptide with a test compound; and
(2) detecting an increased or decreased level of the polypeptide relative to the level of translation obtained in the absence of the test compound.

13. A method for identifying a compound that specifically binds to a polypeptide having an amino acid sequence selected from the group consisting of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA529025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA586761; said method comprising the steps of
(1) providing said polypeptide; and
(2) identifying a compound that is capable of binding said polypeptide.

14. A monoclonal antibody or derivative thereof that specifically binds to a polypeptide having an amino acid sequence selected from the group consisting of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676].

15. A method for identifying a compound that increases or decreases the level in heart tissue of an mRNA encoding a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 (AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
said method comprising the steps of
(1) contacting a DNA giving rise to said mRNA under conditions that would permit transcription of said mRNA with a test compound; and
(2) detecting an increased/decreased level of the mRNA relative to the level of transcription obtained in the absence of the test compound.

16. A transgenic non-human mammal whose somatic and germ cells comprise at least one gene encoding a functional or disrupted polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
that said functional or disrupted polypeptide has been modified, said modification being sufficient to decrease or increase the amount of said functional polypeptide expressed in the heart tissue of said transgenic non-human mammal, wherein said transgenic non-human mammal exhibits a disease of the heart.

17. The transgenic non-human mammal according to claim 16, wherein said disrupted or functional gene was introduced into the non-human mammal or an ancestor thereof, at an embryonic stage.

18. A transgenic non-human mammal according to claim 16 or 17, wherein the modification is inactivation, suppression or activation of said gene(s) or leads to the reduction or enhancement of the synthesis of the corresponding protein(s).

19. A method for identifying a compound that increases or decreases the expression of a polypeptide in heart tissue, the polypeptide being selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
said method comprising the steps of:
(1) contacting a transgenic non-human mammal according to any one of claims 14 to 16 with a test compound, and
(2) detecting an increased or decreased level of expression of said polypeptide relative to the expression in the absence of said test compound.

20. The method according to claim 19, wherein the test compound prevents or ameliorates a disease of the heart in said transgenic non-human mammal.

21. A method for identifying one or a pluratiy of isogenes of a gene coding for a polypeptide selected from the group consisting of: the polypeptide having the amino acid sequence of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676]; said method comprising the steps of
(1) providing nucleic acid coding for said polypeptide or a part thereof; and
(2) identifying a second nucleic acid that (i) has a homology of 60%, preferably at least 80%, especially at least 90%, advantageously at least 99% or (ii) hybridizes in 4×SSC, 0.1 SDS at 45° C. to the nucleic acid molecule encoding said amino acid sequences.

22. A method for identifying one or a plurality of genes whose expression in heart tissue is modulated by inhibiting, decreasing or increasing the expression of a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
or of an mRNA encoding said polypeptide,
said modulation being indicative of a disease of the heart, said method comprising the steps of:
(1) contacting a plurality of heart tissue cells with a compound that inhibits, decreases or increases the expression of said polypeptide under conditions that permit the expression of said polypeptide in the absence of a test compound, and
(2) comparing a gene expression profile of said heart cell in the presence and in the absence of said compound.

23. A method for identifying one or a plurality of genes whose expression in heart tissue is modulated by the inhibition, decrease or increase of the expression of a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution,
or of an mRNA encoding said polypeptide, said modulation being indicative of a disease of the heart, said method comprising the steps of:
(1) providing expression profiles of
(i) a plurality of heart tissue cells from or derived from a heart of a subject suffering from a disease of the heart; and
(ii) a plurality of heart tissue cells from or derived from a subject not suffering from a disease of the heart; and
(2) comparing the expression profiles (i) and (ii).

24. The method of claim 22 further comprising the steps of
(3) determining at least one gene that is expressed at a lower or higher level in the presence of said compound; and
(4) identifying a further compound that is capable of raising or lowering the expression level of said at least one gene.

25. The method of claim 23 further comprising the steps of
(3) determining at least one gene that is expressed at a lower or higher level in said heart tissue cells from or derived from a heart of a subject suffering from a disease of the heart; and
(4) identifying a further compound that is capable of raising or lowering the expression level of said at least one gene.

26. The method of claim 23 further comprising the steps of
(3) determining at least one gene that is expressed at a higher or lower level in said heart tissue cells from or derived from a heart of a subject suffering from a disease of the heart; and
(4) identifying a further compound that is capable of reducing or raising the expression level of said at least one gene.

27. A method for identifying a protein or a plurality of proteins in heart tissue whose activity is modulated by a polypeptide having the amino acid sequence selected from the group consisting of SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
said method comprising the steps of
(1) providing said polypeptide; and
(2) identifying a further protein that is capable of interacting with said polypeptide.

28. The method of any one of claims 12, 13, 15, 19, 20, 22 or 24 to 26, wherein said compound is a small molecule or a peptide derived from an at least partially randomized peptide library.

29. A method of refining a compound identified by the method of any one of claims 12, 13, 15, 19, 20, 22, 24 to 26 or 28;
said method comprising the steps of
(1) identification of the binding sites of the compound and the DNA or mRNA molecule by site-directed mutagenesis or chimeric protein studies;
(2) molecular modeling of both the binding site of the compound and the binding site of the DNA or mRNA molecule; and
(3) modification of the compound to improve its binding specificity for the DNA or mRNA.

30. The method of any one of claims 12, 13, 15, 19, 20, 22, 24 to 26, 28 or 29, wherein said compound is further refined by peptidomimetics.

31. A method of modifying a compound identified or refined by any one of claims 12, 13, 15, 19, 20, 22, 24 to 26, 28 to 30 as a lead compound to achieve
(i) modified site of action, spectrum of activity, organ specificity, and/or
(ii) improved potency, and/or
(iii) decreased toxicity (improved therapeutic index), and/or
(iv) decreased side effects, and/or
(v) modified onset of therapeutic action, duration of effect, and/or
(vi) modified pharmakinetic parameters (resorption, distribution, metabolism and excretion), and/or
(vii) modified physico-chemical parameters (solubility, hygroscopicity, color, taste, odor, stability, state), and/or
(viii) improved general specificity, organ/tissue specificity, and/or
(ix) optimized application form and route
by
(i) esterification of carboxyl groups, or
(ii) esterification of hydroxyl groups with carbon acids, or
(iii) esterification of hydroxyl groups to, e.g. phosphates, pyrophosphates or sulfates or hemi succinates, or
(iv) formation of pharmaceutically acceptable salts, or
(v) formation of pharmaceutically acceptable complexes, or
(vi) synthesis of pharmacologically active polymers, or
(vii) introduction of hydrophylic moieties, or
(viii) introduction/exchange of substituents on aromates or side chains, change of substituent pattern, or
(ix) modification by introduction of isosteric or bioisosteric moieties, or
(x) synthesis of homologous compounds, or
(xi) introduction of branched side chains, or
(xii) conversion of alkyl substituents to cyclic analogues, or
(xiii) derivatisation of hydroxyl group to ketales, acetates, or
(xiv) N-acetylation to amides, phenylcarbamates, or
(xv) synthesis of Mannich bases, imines, or
(xvi) transformation of ketones or aldehydes to Schiff's bases, oximes, acetates, ketales, enolesters, oxazolidines, thiozolidines
or combinations thereof.

32. A method for inducing a disease of the heart in a non-human mammal, comprising the step of contacting the heart tissue of said mammal with a compound that inhibits, decreases or increases the expression of a polypeptide selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution.

33. The method according to claim 32, wherein said compound that inhibits, decreases or increases is a small molecule, an antibody or an aptamer that specifically binds said polypeptide.

34. A method of producing a pharmaceutical composition comprising formulating the compound identified, refined or modified by the method of any of the preceding claims with a pharmaceutically active carrier or diluent.

35. A method for preventing or treating a disease of the heart in a subject in need of such treatment, comprising the step of increasing or decreasing the level of a polypeptide in the heart tissue of a subject, said polypeptide being selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution.

36. A method of preventing or treating a disease of the heart in a subject in need of such treatment comprising the step of increasing or decreasing the level of mRNA encoding a polypeptide in the heart tissue of a subject, said polypeptide being selected from the group consisting of:
(a) the polypeptide having amino acid sequence SEQ ID NO: 1 [NP_{—}003961], the amino acid sequence of SEQ ID NO: 2 [41441pep], the amino acid sequence of SEQ ID NO: 3 [56461pep], the amino acid sequence of SEQ ID NO: 4 [AAA52025], the amino acid sequence of SEQ ID NO: 5 [61166pep], the amino acid sequence of SEQ ID NO: 6 [AAD45360], the amino acid sequence of SEQ ID NO: 7 [AAF63623], the amino acid sequence of SEQ ID NO: 8 [66214pep] or the amino acid sequence AAF19343, or the amino acid sequence of SEQ ID NO: 9 [CAA58676];
(b) a polypeptide having an amino acid sequence that is at least 60%, preferably at least 80%, especially at least 90%, advantageously at least 99% identical to the amino acid sequence of (a); and
(c) a polypeptide having the amino acid sequence of (a) with at least one conservative amino acid substitution.

37. The method of claims 35 or 36, wherein such increase or decrease is effected by administering the pharmaceutical composition obtained by the method of claim 30.

38. The method of claim 35 or 36, wherein such an increase or decrease is effected by introducing the nucleic acid sequence recited in claim 2 into the germ line or into somatic cells of a subject in need thereof.

39. The method of any of the preceding claims, wherein said disease of the heart is congestive heart failure, dilative cardiomyopathy, hypertrophic cardiomyopathy, ischemic cardiomyopathy, specific heart muscle disease, rhythm and conduction disorders, syncope and sudden death, coronary heart disease, systemic arterial hypertension, pulmonary hypertension and pulmonary heart disease, valvular heart disease, congenital heart disease, pericardial disease or endocarditis.

40. A method for identifying subjects at risk for heart diseases, especially congestive heart failure, comprising the step of detecting an increased or decreased level of MYOM2, the LIM domain, the muscle isoform of creatine kinase, YAP65, APOBEC-2, SMPX or C-193 (CARP) in the heart tissue of a subject.

41. A method for preventing or treating heart diseases, especially congestive heart failure in a subject, said method comprising the step of contacting the heart tissue of said subject with a compound that decreases or increases the expression of MYOM2, the LIM domain, the muscle isoform of creatine kinase, YAP65, APOBEC-2, SMPX or C-193 (CARP).

42. A method for identifying subjects at risk for heart diseases, especially congestive heart failure, comprising the step of detecting decreased creatine kinase activity in the tissue of a subject especially in a muscle tissue or from blood or serum.

43. A method for identifying a subject at risk for heart diseases, especially congestive heart failure, said method comprising detecting increased levels of creatine phosphate in a subject, especially in the blood or serum of a subject.

44. A method for preventing or treating heart diseases, especially congestive heart failure in a subject, said method comprising the step of increasing the transfer of phosphoryl groups from creatine phosphate to ADP in the heart tissue of a subject.

45. The method according to claim 44, wherein the activity of creatine kinase is increased in said heart tissue.

46. A method for identifying a compound for preventing or treating heart diseases, especially congestive heart failure, said method comprising the steps of:
(a) contacting creatine kinase with a substrate for creatine kinase and a test compound, and
(b) determining whether the transfer of phosphoryl groups from the substrate is increased in the presence of the test compound.

47. Use of a compound of one of the claims 12, 13, 15, 19, 20, 24 to 26, 28, 41, 46, a refined or modified compound of one of the claims 29, 30 or 31, or a monoclonal antibody of the claim 14 for the manufacture of a pharmaceutical composition for the prophylaxis or treatment of heart diseases, especially congestive heart failure.


SEQUENCE LISTING (38 sequences)

SEQ ID NO: 1 - protein, 1465 aa, Homo sapiens
MSLVTVPFYQKRHRHFDQSYRNIQTRYLLDEYASKKRASTQASSQKSLSQRSSSQRASSQTSLGGTICRVCAKRVSTQEDEEQENRSRYQSLVAAYGEAKRHGFLSELAHLEEDVHLARSQARDKLDKYAIQQMMEDKLAWERHTFEERISRAPEILVRLRSHTVWERMSVKLCFTVQGFPTPVVQWYKDGSLICQAAEPGKYRIESNYGVHTLEINRADFDDTATYSAVATNAHGQVSTNAAVVVRRFRGDEEPFRSVGLPIGLPLSSMIPYTHFDVQFLEKFGVTFRREGETVTLKCTMLVTPDLKRVQPRAEWYRDDLLLKESKWTKMFFGEGQASLSFSHLHKDDEGLYTLRIVSRGGVTDHSAFLFVRDADPLVTGAPGAPMDLQCHDANRDYVIVTWKPPNTTTESPVMGYFVDRCEVGTNNWVQCNDAPVKICKYPVTGLFEGRSYIFRVRAVNSAGISRPSRVSDAVAALDPLDLRRLQAVHLEGEKEIAIYQDDLEGDAQVPGPPTGVHASEISRNYVVLSWEPPTPRGKDPLMYFIEKSVVGSGTWQRVNAQTAVRSPRYAVFDLMEGKSYVFRVLSANRHGLSEPSEITSPIQAQDVTVVPSAPGRVLASRNTKTSVVVQWDRPKHEEDLLGYYVDCCVAGTNLWEPCNHKPIGYNRFVVHGLTTGEQYIFRVKAVNAVGMSENSQESDVIKVQAALTVPSHPYGITLLNCDGHSMTLGWKVPKFSGGSPILGYYLDKREVHHKNWHEVNSSPSKPTILTVDGLTEGSLYEFKIAAVNLAGIGEPSDPSEHFKCEAWTMPEPGPAYDLTFCEVRDTSLVMLWKAPVYSGSSPVSGYFVDFREEDAGEWITVDQTTTASRYLKVSDLQQGKTYVFRVRAVNANGVGKPSDTSEPVLVEARPGTKEISAGVDEQGNIYLGFDCQEMTDASQFTWCKSYEEISDDERFKIETVGDHSKLYLKNPDKEDLGTYSVSVSDTDGVSSSFVLDPEELERLMALSNEIKNPTIPLKSELAYEIFDKGRVRFWLQAEHLSPDASYRFIINDREVSDSEIHRIKCDKATGIIEMVMDRFSIENEGTYTVQIHDGKAKSQSSLVLIGDAFKTVLEEAEFQRKEFLRKQGPHFAEYLHWDVTEECEVRLVCKVANTKKETVFKWLKDDALYETETLPNLERGICELLIPKLSKKDHGEYKATLKDDRGQDVSILEIAGKVYDDMILAMSRVCGKSASPLKVLCTPEGIRLQCFMKYFTDEMKVNWCHKDAKISSSEHMRIGGSEEMAWLQICEPTEKDKGKYTFEIFDGKDNHQRSLDLSGQAFDEAFAEFQQFKAAAFAEKNRGRLIGGLPDVVTIMEGKTLNLTCTVFGNPDPEVIWFKNDQDIQLSEHFSVKVEQAKYVSMTIKGVTSEDSGKYSINIKNKYGGEKIDVTVSVYKHGEKIPDMAPPQQAKPKLIPASASAAGQ

SEQ ID NO: 2 - DNA, 403 bp, Homo sapiens
aagaagaagagatgtgcaaggataggccgagtgaagctgaagacacaaagagtacaggaaaagtgctatggatcttaatgacaacaataatgtgattgtgcagagtgctgaaaaggagaaaaatgaaaaaactaaccaaactaatggtgcagaagttttacaggttactaacactgatgatgagatgtgccagaaaatcataaagaaaatttgaataagaataataataacaattatgtagcagtctcatatctgaataattgcaggcagaagacatctattttagaatttcttgatctattacccttgtcgagtgaagcaaatgacactgcaaatgaatatgaaattgagaagttagaaaatacatctagaatctcagagttacttggtagatttgaatctg

SEQ ID NO: 3 - protein, 142 aa, Homo sapiens
LHSLGGGDQSHVMSVVRSSVHARWIVGKVIGTKMQKTAKVRVTRLVLDPYLLKYFNKRKTYFAHDALQQCTVGDIVLLRALPVPRAKHVKHELAEIVFKVGKVIDPVTGKPCAGTTYLESPLSSETTQLSKNLEELNISSAQ

SEQ ID NO: 4 - protein, 381 aa, Homo sapiens
MPFGNTHNKFKLNYKPEEEYPDLSKHNNHMAKVLTLELYKKLRDKEIPSGFTVDDVIQTGVDNPGHPFIMTVGCVAGDEESYEVFKELFDPIISDRHGGYKPTDKHKTDLNHENLKGGDDLDPNYVLSSPVRTGRSIKGYTLPPHCSRGERRAVEKLSVEALNSLTGEFKGKYYPLKSMTEKEQQQLIDDHFQFDKPVSPLLLASGMARHWPDAPGIWHNDNKSFLVWVNEEDHLRVISMEKGGNMKEVFRRFCVGLQKIEEIFKKAGHPFMWNQHLGYVLTCPSNLGTGLRGGVHVKLAHLSKHPKFEEILTRLRLQKRGTGAVDTAAVGSVFDVSNADRLGSSEVEQVQLVVDGVKLMVEMEKKLEKGQSIDDMIPAQK

SEQ ID NO: 5 - protein, 398 aa, Homo sapiens
MNPASAPPPLPPPGQQVIHVTQDLDTDLEALFNSVMNPKPSSWRKKILPESFFKEPDSGSHSRQSSTDSSGGHPGPRLAGGAQHVRSHSSPASLQLGTGAGAAGSPAQQHAHLRQQSYDVTDELPLPPGWEMTFTATGQRYFLNHIEKITTWQDPRKAMNQPLNHMNLHPAVSSTPVPQRSMAVSQPNLVMNHQHQQQMAPSTLSQQNHPTQNPPAGLMSMPNALTTQQQQQQKLRLQRIQMERERIRMRQEELMRQEAALCRQLPMEAETLAPVQAAVNPPTMTPDMRSITNNSSDPFLNGGPYHSREQSTDSGLGLGCYSVPTTPEDFLSNVDEMDTGENAGQTPMNINPQQTRFPDFLDCLPGTNVDLGTLESEDLIPLFNDVESALNKSEPFLT

SEQ ID NO: 6 - protein, 224 aa, Homo sapiens
MAQKEEAAVATEAASQNGEDLENLDDPEKLKELIELPPFEIVTGERLPANFFKFQFRNVEYSSGRNKTFLCYVVEAQGKGGQVQASRGYLEDEHAAAHAEEAFFNTILPAFDPALRYNVTWYVSSSPCAACADRIIKTLSKTKNLRLLILVGRLFMWEEPEIQAALKKLKEAGCKLRIMKPQDFEYVWQNFVEQEEGESKAFQPWEDIQENFLYYEEKLADILK

SEQ ID NO: 7 - protein, 264 aa, Homo sapiens
MLSHNTMMKQRKQQATAIMKEVHGNDVDGMDLGKKVSIPRDIMLEELSHLSNRGARLFKMRQRRSDKYTFENFQYQSRAQINHSIAMQNGKVDGSNLEGGSQQAPLTPPNTPDPRSPPNPDNIAPGYSGPLKEIPPEKFNTTAVPKYYQSPWEQAISNDPELLEALYPKLFKPEGKAELPDYRSFNRVATPFGGFEKASRMVKFKVPDFELLLLTDPRFMSFVNPLSGRRSFNRTPKGWISENIPIVITTEPTDDTTVPESEDL

SEQ ID NO: 8 - protein, 88 aa, Homo sapiens
MYMSKQPVSNVRAIQANINIPMGAFRPGAGQPPRRKECTPEVEEGVPPTSDEEKKPIPGAKKLPGPAVNLSEIQNIKSELKYVPKAEQ

SEQ ID NO: 9 - protein, 319 aa, Homo sapiens
MMVLKVEELVTGKKNGNGEAGEFLPEDFRDGEYEAAVTLEKQEDLKTLLAHPVTLGEQQWKSEKQREAELPKKKLEQRSKLENLEDLEIIIQLKKRKKYRKTKVPVVKEPEPEIITEPVDVPTFLKAALENKLPVVEKFLSDKNNPDVCDEYKRTALHRACLEGHLAIVEKLMEAGAQIEFRDMLESTAIHWASRGGNLDVLKLLLNKGAKISARDKLLSTALHVAVRTGHYECAEHLIACEADLNAKDREGDTPLHDAVRLNRYKMIRLLIMYGADLNIKNCAGKTPMDLVLHWQNGTKAIFDSLRENSYKTSRIATF

SEQ ID NO: 10 - DNA, 4939 bp, Homo sapiens
ttctctctcctccttgcaattttcctttctgtctgggagcacgccaagatgtcccttgtgactgtccccttctaccagaagagacataggcacttcgaccagtcctaccgtaatattcaaacacggtacctgctggacgaatatgcgtcaaaaaagcgagcttccacccaggcatcttcccagaagtccttgagtcagcggtcgtcttcacagagagcctccagccagacgtccctgggaggaaccatctgcagggtctgtgcgaagcgagtgagcacgcaggaagatgaggagcaggagaacagaagcaggtaccagtccctggtggccgcctatggtgaggccaagcgacacggcttcctcagcgagctggcccacttggaggaggatgtccacctggcacgctcccaggcccgcgacaagctggacaaatacgccattcagcagatgatggaggacaagctggcctgggagagacacacatttgaagagcggataagcagggctcctgagatcctggtgcggctgcgatcccacaccgtctgggagaggatgtctgtgaaactctgcttcaccgtgcaaggatttcccacgcccgtggtgcagtggtacaaagatggcagtctgatttgccaggcggctgaaccgggaaagtacaggattgagagcaactatggcgtacacacactggagatcaacagggcagactttgacgacactgcgacatactcagcagtggccaccaatgcccacggacaagtgtccaccaacgcggcggtggtggtgagaaggttccggggagacgaggaaccattccgttcggtgggactcccgattggattgcccctgtcatcgatgattccgtacacgcacttcgacgtccagtttttggagaagtttggggtcaccttcaggagggaaggcgagacggtcactctcaagtgcaccatgctggtgacgccggacctgaagcgggtgcagccgcgcgccgagtggtaccgcgatgacttgctgttgaaagagtccaagtggacgaagatgttctttggagaaggccaggcctccctgtccttcagccacctgcacaaggacgacgagggcctgtacaccctgcgcatcgtgtctcggggcggcgtcacggaccacagcgccttcctgtttgtcagagatgctgacccgctggtcacaggggcccccggtgcacccatggacttgcagtgccacgacgccaaccgggactacgtcatcgtgacctggaagccgcccaacaccaccactgagagccccgtcatgggctattttgtggaccgatgtgaagtaggaacgaataattgggtgcagtgcaatgatgcaccggtgaaaatctgcaaatacccggtcacagggctttttgaaggaaggtcttacatattccgagtgagggcagtgaacagtgcgggcatcagccgaccctccagggtctctgatgcggtggctgcacttgaccccttggacctcagaaggttacaagccgttcatttggagggagagaaggagattgccatttatcaggatgaccttgaaggtgacgcccaggttccagggcctcccaccggtgtgcacgcttccgagatcagcagaaactatgtcgtcctcagctgggagccacccactccccgtggcaaggacccgctcatgtacttcattgagaagtcggtggtggggagcggcacgtggcagagagtcaacgcccagacggctgtgagatccccgagatatgccgtgtttgacctcatggaagggaagtcttatgtgttccgagtgctgtcagcaaaccggcatggcctgagcgaaccttcggagataacgtcccccattcaggcccaggatgtgaccgttgtcccttctgctccgggtcgggttcttgcttcccgaaacaccaagacgtcggtggtggtgcagtgggaccgacctaagcatgaggaggacctgctgggctactacgtggactgctgtgtggccggaaccaacctctgggagccctgcaaccacaagcccatcggatacaacaggttcgtggtgcacggcttaaccacgggagagcagtacatcttccgagtcaaggcggtcaatgctgtggggatgagtgaaaattcccaggaatcagacgtcataaaagtgcaggccgcactcaccgtcccgtcccatccttatgggattacgctcctcaactgtgacggccactccatgaccctcggctggaaggtcccgaaattcagtggtggctcgcccatcctgggctactacctggacaagcgtgaagttcaccataaaaactggcacgaggtcaattcctcacccagcaaaccgacaatcctaacggtggacggcttgacggaaggctcactctacgagttcaaaatcgccgccgtcaacctggccggcatcggggagccctcagatcccagtgagcacttcaagtgtgaggcctggaccatgccggagcccggtcctgcctacgacttgacgttctgtgaggtcagggacacgtccttggtcatgctgtggaaggcccctgtgtactccggcagcagccctgtttctggatatttcgtggacttcagggaggaggatgctggagagtggatcactgtcgatcagacgacaacagccagccgttatttaaaggtctctgacctgcagcaaggtaagacctatgtcttcagggtccgggcagtcaatgcaaatggcgtggggaagccctcagacacgtcggagcctgtgctggtagaggcgagaccaggcaccaaggaaatcagtgctggtgtcgatgaacagggcaacatctatctgggcttcgactgccaggaaatgacagacgcgtctcagttcacctggtgtaaatcctacgaggagatttcagatgatgagaggtttaaaatcgaaaccgtgggggatcactccaagctgtacttaaagaatccggataaggaggatttagggacttactccgtgtctgtaagtgatacagacggagtgtcctccagttttgttctggacccagaagagctcgagcgtttgatggcattgagcaatgaaataaagaaccccacaattcctctgaaatcggaattagcttatgagatttttgataaggggcgggttcgcttctggctccaggctgagcacttatcaccagatgccagctaccgatttattattaatgacagagaagtctctgacagcgagatacacagaattaaatgtgacaaagctactggcattattgagatggtgatggatcgatttagtattgaaaatgaggggacctacactgtgcagattcatgatgggaaagccaaaagtcagtcttctctagttcttattggagatgcattcaagactgtgctggaagaggctgagtttcaaaggaaagaatttctcaggaaacaaggccctcattttgctgagtacttgcactgggatgtcacggaagaatgtgaagttcgacttgtttgcaaggttgcaaacaccaagaaagaaaccgttttcaaatggctcaaggatgatgctctgtatgaaacggagacactgcctaacctggagaggggaatctgtgagctcctcatcccaaagttgtcaaagaaggaccacggtgaatacaaggcaaccttgaaagatgacagaggccaagatgtgtccatccttgaaatagctggcaaagtgtatgatgatatgattttggcaatgagtagagtctgtgggaaatctgcttcgccactgaaggtactctgcaccccagaaggaatacgacttcagtgtttcatgaagtattttacagacgaaatgaaagtgaactggtgtcacaaagatgctaagatctcatccagtgagcatatgagaatcggggggagtgaagagatggcttggctgcagatatgtgagccgactgagaaggataaaggaaaatacacttttgagattttcgatggcaaagacaaccatcaacgctcccttgacctgtccggacaagcttttgatgaagcatttgcagaattccagcaattcaaagctgctgcttttgcagagaagaatcgtggcaggttgatcggcggcttgcctgacgtggtgaccatcatggaagggaagaccttgaatctgacctgcacggtgtttggaaaccctgaccccgaagtgatttggttcaagaacgaccaggacatccagctcagcgagcacttctcggtgaaggtggagcaggccaagtacgtcagcatgaccatcaaaggcgtgacctccgaggactcgggcaagtacagcatcaacatcaagaataagtatggcggggagaagatcgacgtgacggtgagcgtgtacaaacacggggagaagatcccggacatggccccgccccagcaagccaagcccaagctcatccccgcgtctgcctcagcggcaggccagtgaaggcgttttcctagcctggagatgggaaaatatgcttggcagagacaggaatgctgtgtgcttgttccaaatgagcagctggcatccgagtggtgtcctgtgtgggctgatagttgatcacacattgtgcttttgatttttgcatttggtgatgaatattttatacccgtctaagggagaaagctaatgttttccacaagactgaacaacgtgtatttacacgagggtagacggcagatgcctgacagagagtgggttggcagacaacacactagcattttcacgggtgtgggcacatgggtgtggcacctggacgtgtgcagcatgtggcggtctctgtgtgaagccaccgtgcttctctttggggggccgcgagatctagcatctctgaaatcctggctgtcgaggctttgaagcatgtgttacctggttaagcttgttttctcttgctttaggcaaataaaagtttaaaaatcaaaaaaaaaaaaaaaaa

SEQ ID NO: 11 - DNA, 2379 bp, Homo sapiens
cccaggatctgctctgaaaccaggtctctaagtgaacatttctcaggcatggatgcatttgagagtcaaattgttgagtcgaagatgaaaacctcttcatcacatagctcagaagctggcaaatctggctgtgacttcaagcatgccccaccaacctatgaggatgtcattgctggacatattttagatatctctgattcacctaaagaagtaagaaaaaattttcaaaagacgtggcaagagagtggaagagtttttaaaggcctgggatatgcaaccgcagatgcttctgcaacatgagatgagaaccaccttccaagaggaatctgcatttataagtgaagctgctgctccaagacaaggaaatatgtatactttgtcaaaagacagtttatccaatggagtgcctagtggcagacaagcagaattttcataagtcctgcttccgatgccaccattgcaacagtaaactaagtttgggaaattatgcatcacttcatggacaaatatactgtaaacctcactttaaacaacttttcaaatccaaaggaaattatgatgaaggttttggacataagcagcataaagatagatggaactgcaaaaaccaaagcagatcagtggactttattcctaatgaagaaccaaatatgtgtaaaaatattgcagaaaacacccttgtacctggagatcgtaatgaacatttagatgctggtaacagtgaagggcaaaggaatgatttgagaaaattaggggaaaggggaaaattaaaagtcatttggcctccttccaaggagatccctaagaaaaccttaccctttgaggaagagctcaaaatgagtaaacctaagtggccacctgaaatgacaaccctgctatcccctgaatttaaaagtgaatctctgctagaagatgttagaactccagaaaataaaggacaaagacaagatcactttccatttttgcagccttatctacagtccacccatgtttgtcagaaagaggatgttataggaatcaaagaaatgaaaatgcctgaaggaagaaaagatgaaaagaaggaaggaaggaagaatgtgcaagataggccgagtgaagctgaagacacaaagagtaacaggaaaagtgctatggatcttaatgacaacaataatgtgattgtgcagagtgctgaaaaggagaaaaatgaaaaaactaaccaaactaatggtgcagaagttttacaggttactaacactgatgatgagatgatgccagaaaatcataaagaaaatttgaataagaataataataacaattatgtagcagtctcatatctgaataattgcaggcagaagacatctattttagaatttcttgatctattacccttgtcgagtgaagcaaatgacactgcaaatgaatatgaaattgagaagttagaaaatacatctagaatctcagagttacttggtatatttgaatctgaaaagacttattcgaggaatgtactagcaatggctctgaagaaacagactgacagagcagctgctggcagtcctgtgcagcctgctccaaaaccaagcctcagcagaggccttatggtaaaggggggaagttcaatcatctctcctgatacaaatctcttaaacattaaaggaagccattcaaagagcaaaaatttacactttttcttttctaacaccgtgaaaatcactgcattttccaagaaaaatgagaacattttcaattgtgatttaatagattctgtagatcaaattaaaaatatgccatgcttggatttaagggaatttggaaaggatgttaaaccttggcatgttgaaacaacagaagctgcccgcaataatgaaaacacaggttttgatgctctgagccatgaatgtacagctaagcctttgtttcccagagtggaggtgcagtcagaacaactcacggtggaagagcagattaaaagaaacaggtgctacagtgacactgagtaaaatatctatggccactgacagtccacacttaggcactgagagatattgatgttctgaaataagattttatgaatttggatacccttttgaggaacttgatgtaaacatggtgttcagaaatctcgtgtctatctcaatgggatatttcttgtattacaccttgtcatttttttcacaatttatttacatctacttttgtttgaactggaatgaagagatgaaacactatggatatgttttccattcaaatggcactttagcatattgttctgttttcctgtaaaacatcatgggtgtgatttttatactgctgctgcttgtcacaattattataacttctctgtaatttcctctgaaataaaattgaatcacctgaggtgcaaaccaaaaaaaaaaa

SEQ ID NO: 12 - DNA, 125 bp, Homo sapiens
acagcttacagaactgtgggccaaataaacctcttttctttataaattacccagcttcagatattcctttatagcgacacaaatggactaaggtgtcaagatcatttgatagagaaaggcattgt

SEQ ID NO: 13 - DNA, 508 bp, Homo sapiens
ggctgcatagtcttggcggaggtgaccaaagccgcgtaatgtccgtagttcgctcatccgtccatgccagatggattgtggggaaggtgattgggacaaaaatgcaaaagactgctaaagtgagagtgaccaggcttgttctggatccctatttattaaagtattttaataagcggaaaacctactttgctcacgatgcccttcagcagtgcacagttggggatattgtgcttctcagagctttacctgttccacgagcaaagcatgtgaaacatgaactggctgagatcgttttcaaagttggaaaagtcatagatccagtgacaggaaagccctgtgctggaactacctacctggagagtccgttgagtcggaaaccacccagctaagcaaaaatctggaagaactcaatatctcttcagcacagtgaagcgggagtgaagaaggatctaaagggaaaaactgacatgtttatgttatggaaaaagaaatttttctaagtttcatc

SEQ ID NO: 14 - DNA, 1562 bp, Homo sapiens
gtgggtcagcatgtcacctccaggatacagacagccccccttcagcccagcccagccaggtctccttacaccgccaccatgccattcggtaacacccacaacaagttcaagctgaattacaagcctgaggaggagtaccccgacctcagcaaacataacaaccacatggccaaggtactgacccttgaactctacaagaagctgcgggacaaggagatcccatctggcttcactgtagacgatgtcatccagacaggagtggacaacccaggtcaccccttcatcatgaccgtgggctgcgtggctggtgatgaggagtcctacgaagttttcaaggaactctttgaccccatcatctcggatcgccacgggggctacaaacccactgacaagcacaagactgacctcaaccatgaaaacctcaagggtggagacgacctggaccccaactacgtgctcagcagcccggtccgcactggccgcagcatcaagggctacacgttgcccccacactgctcccgtggcgagcgccgggcggtggagaagctctctgtggaagctctcaacagcctgacgggcgagttcaaagggaagtactaccctctgaagagcatgacggagaaggagcagcagcagctcatcgatgaccacttccagttcgacaagcccgtgtccccgctgctgctggcctcaggcatggcccgccactggcccgacgcccctggcatctggcacaatgacaacaagagcttcctggtgtgggtgaacgaggaggatcacctccgggtcatctccatggagaaggggggcaacatgaaggaggttttccgccgcttctgcgtagggctgcagaagattgaggagatctttaagaaagctggccaccccttcatgtggaaccagcacctgggctacgtgctcacctgcccatccaacctgggcactgggctgcgtggaggcgtgcatgtgaagctggcgcacctgagcaagcaccccaagttcgaggagatcctcacccgcctgcgtctgcagaagaggggtacaggtgcggtggacacagctgccgtgggctcagtatttgacgtgtccaacgctgatcggctgggctcgtccgaagtagaacaggtgcagctggtggtggatggtgtgaagctcatggtggaaatggagaagaagttggagaaaggccagtccatcgacgacatgatccccgcccagaagtaggcgcctgcccacctgccaccgactgctggaaccccagccagtgggagggcctggcccaccagagtcctgctccctcactcctcgccccgccccctgtcccagagtccacctgggggctctctccacccttctcagagttccagtttcaaccagagttccaaccaatgggctccatcctctggattctggccaatgaaatatctccctggcagggtcctcttcttttcccagagctcctccccaaccaggagctctagttaatggagagctcccagcacactcggacgcttgtgcttttgtctccacgcaaacggataaataaaagcattggtggcctt

SEQ ID NO: 15 - DNA, 4828 bp, Homo sapiens
ttttccaagtggaagttcaattgtctttatttttcttatacagattcagagaagtaaaaaccagtaccaaactccaggtaaaatggtttgatctgatcgatttggctgcatactttcggtacgtataacattctaaacttaaaatagaaatttttatattacaaaacgtagaagtaaaattttaaaaagttaaagtactagcacatatatgtgttaggaaaatggtctctgtcaattgcccattttcccaattaaattaacctacgatttcctttttttaacagcttatttttttcataaaagttgtactttgagaagttactttctaattacgtcatgagaacacaacttgtaattagcaacacttctgtcagtctagatcacttcttctgcagagagcttttcaaccaagttggcatcaaccagcacaataaagttttcactgttttacctgtttcctgtatatggtgtaatcagtgaaagaaatggcatttcacatcctaaataatacggtgaaacactgtctaaaaattacttagatttaacagaattgcaattaggttttgacaatgtatttacttcaagacaatgtattttatcaggaaaaaatatcttgaaagaaagatctctgaaattatttttcatttgatacgccttttctgtgacaaaattttggggtgaaatgatgatgtttgctgattgatttagtactaaaaagactagtactaagaagactaaagacagttatcttataataagaaatatagtataaatagcaccttatcaagaattctgcaggggttttaacacttacaataataggaaatagccattaaaaagttgctctaactttagatttctaactttagtgttctttaacaaaggccatattttgtggccttaaaaacaaaaaattatatctggctttatctattagtaaacacaaagggtccatattttattctgaaaaaatatttattatattcattcataaatgttctaactaatttaactaaaaaaatcttctagtattttctgatgccacaagcttactagaaaattacttctaaaaattggtaatataaatcatcaatgatttacctactttaaaaaagaggggtatctgtttctcttacatttaataacctgaaaatgagtctataaaaatattttaaaaaaatacagtaacactgctgagttttgttaggtcccttgtttttttaattttttatttatttatttatttttagcaagaatgtacaattctttttgcaattttttgctaacaaaagacaaaaagaaatagtgctcccttcaatttagtagcaataaaatcatctatcttcatctctctcagagggcttagggagagtgaaaggaattagaggaacataaaccatgggtccttcaggtaaaataagtcatttcatagtgatggaggcaacagcaggctacgctcttgtctgcctgtacgctcagcatcagcatgccctgcccctcctcctcccacataaggtgcaccaaagccttcttttcttctacaacagagtcttccggtataaaggtggcaacagaaaattgttgctttgtccttttggggcatctcttgcatctcctgcttgttttgcttttggggtactataacttgtctctttccatttcacaactagtatcctttccacgatattccataacttcgctacttcatctctgaccttttcacttgccttctttccaacatgatccaatgtactctgtacatgtatattccggtagatcaaaaggaatcttatttaagatccccaacaaataagtcccccatggaaattgaaagtatcctctcagagactcaaattattaaagtctttcaaaaaagattaaattcatagattataaataatattagttcaaaatattaaacagttgaggacttcattggcaatgcaggcagactgcatgccagttgaacatgatgctctctcagtccttaaaagctaattaaaaatggttttggttacataagaggtattgaatacatatttcatgcctttttataccaactgtagcaaacaggattaggataatatacttaggaatcaattttactgaattcagaaacatttatctccaccatacaccctcaaagggcatttttttttacatgtcagtcagagatctgcttcatccttcagtttcatagatagaattattttaaacacttgaaatctaggaagcaaacctgacaaggcttcagaatttaaaaagcaacagctcactgtgtgtggtgtgctatcaggttgaaatctatgttgtcctgatgttttcagagttatttcaaaagacaaaaatacagttgccactgatttatcaaaaacatttggctgccttttgtcatcagctacaaaattacagtgctttataaaataaacatcaaggccgggtgcggtggctcacgccagtaatcccagcactttggaaggccgagatgggtggatcacctgagatcaggagtttgagaacagcctgaccattatggtgaaaccccgtctctactcaaaatacaaaaaattagccggacgtggtggcaggcgcctgtaatcccagctactcgggaggctaaggcaggagaatcgcttgaacccagaaggcagaggttgcagtgagccgagattgtgccattgcattccagcctggacaacaagagcaaaactcgatctcaaaaacaaacaaacaacatcaagaaaaaaaaaaaaccatcagattctaagctgcaattttttaaatccccagttgtaatatttcaaaaacttttgtttgaataaaatgctcataattagtaccaaactggtctctttcacaagatctgtagtgtaagaactgtgactaatgctgctgctgctactttcacgctggagggactaagttgtctagatgctctgcagtaaaataacgaaagataagctacaataggactgtgtgcctttataaatacagactaataagagccatcagagccagcatggattcaaaattacattgtattccatacagtagaattttactatccatacaatgatttttaaagctcaagttaaatagtttttaaagcatttggtactactgtcatcaatacagtttttgaaactgtaaatcaggtcgaattttgtgcacatttcctggaccaagatgccctcagaagtaactgcctgtggtcagctttttatggtttaaaatcaattggtgtataaatttcaattaacacccataaagcttagccatggggcagcagagaagaaagagaaaagtattctgcataatcaatcctgcagacacaattctgtataatctgtcacaagaacgcaggcttgcagaaaatgaaaatagaatatttatttatgtttaacttaagttactctcaatcaaaaccaggcaatgattaaactggcaacataaaaaggagggagcacgagtcatggaggcgggaagtggtgcacctgcagacttgctctgctccatcactttttccaagaggcccaggaaatgtaaggtcatggctacatccaagttacaatggtagtgattacagccaggttagaaagggctcacttttgttcagagcagactctacatcattgaagagggggatcaggtcttcagattccaaagttcctaagtcaacgtttgttcctggaagacagtcaaggaaatcagggaaacgggtctgttggggattgatgttcatgggtgtttgtcctgcgttttctcctgtatccatctcatccacattgctgaggaagtcctccggagttgtggggacactgtagcaccctaaccccaggccactgtcagtgctctgctccctcgaatgatatggccctccattgaggaaaggatctgagctattattagtgatggatctcatgtctggggtcatcgtgggtgggttgacagcagcctgaactggggcaagagtctcagcttccatggggagctgtcgacagagggcagcttcctgcctcatgagctcctcttggcgcattcgaatcctttctctctacatctggattctctgaagccgcagtttctgctgctgctgctgctgagtggtcagcgcattgggcatactcatgagccctgcgggtgggttctgagtggggtggttctgctggctcagggtactgggggccatctgctgctggtgttggtgattcatcacgagatttggctgggatactgccatggacctctgaggcactggtgtggaactgacggcagggtggaggttcatatgattcagaggctgattcatcgccttcctagggtcttgccatgtggtgattttttctatgtgattgaggaagtacctctggccagtggccgtgaaggtcatctcccagcccgggggcagtggcagctcgtcggtcacgtcgtaggactgctggcggaggtgcgcgtgctgctgcgcggggctacccgcagcacccgcgccggtgcccagctgcagggacgcgggcgacgagtgcgagcggacatgctgggcacccccagccagtcgaggccccgggtggccgcccgacgagtcggtgctggactggcgcgagtgcgagcccgaatcaggctccttaaagaaagactccggcaggatcttcttccgccacgagctaggcttcggattcatgacagagttgaagagggcttcgaggtctgtgtctaggtcctgcgtgacgtggatcacttgctgcccaggcggcgggagcggagggggcgccgaggccggattcatcttctgggcgggcagcgaagctgagcctgagcgcgcggcggccgc

SEQ ID NO: 16 - DNA, 1164 bp, Homo sapiens
gaattccggcggcctctctcctctccctcagtgactcctgagccacagcccctccatggcccagaaggaagaggctgctgtggccactgaggctgcctcccagaatggggaggatctggagaacctggacgaccctgagaagctgaaagagctgattgagctgccgccctttgagattgtcacaggagaacggctgcctgccaacttctttaaattccagttccggaatgtggagtacagttccgggaggaacaagaccttcctctgctatgtggttgaagcacagggcaaggggggccaagtgcaggcatctcggggatacctagaggatgagcatgcggctgcccatgcagaggaagctttcttcaacaccatcctgccagccttcgacccagccctgcggtacaatgtcacctggtatgtgtcctccagcccctgtgcagcgtgtgctgaccgcattatcaaaacccttagcaagaccaagaacctgcgtctgctcattctggtgggtcgactcttcatgtgggaggagccggagatccaggctgctctgaagaagctgaaggaggctggctgtaaactgcgcatcatgaagccccaggacttcgaatatgtctggcagaattttgtggagcaagaagagggtgaatccaaggcctttcagccctgggaggacattcaggagaacttcctatactacgaggagaagttggcagacatcctgaagtagggcaactgggctttgcctcacgtattcctgctgccaccaagagacagcaatgacatgtacagccatctgggacatgcctgtcttcctaataccatttggagctggacaacatttgacaccaaccaatcatactggacaaggcccttagaggacttgaaatatacttctcatgctgtagtttatttaggctgtgactctctctctaatgctgctctcgggaaggacgaaagtgacctgcaaggagagaaatgcaaccatacatgggctccagtcaactatgggactgaaggtcctaattgctcacccaagggggctgcttaacacaaacagcctcagacccgaggtttagatttctgaaatatgcattttatgttaagttgggtatttttttaaaaaaagaaaaacagcaacattaataaaagaagtggtgtggc

SEQ ID NO: 17 - DNA, 1590 bp, Homo sapiens
  misc_feature 1492, 1529, 1586: n = A,T,C or G
gtcagagtagggaccatgctgtcccaggttcaaggataaaaaccatcaggcccaagtgccatccatagtccatctccagagtcttcctccacaaactgggattcatccccgctgaaaaagcacaatctaacagcaagggaacaaaaaaaccatgctatcacataatactatgatgaagcagagaaaacagcaagcaacagccatcatgaaggaagtccatggaaatgatgttgatggcatggacctgggcaaaaaggtcagcatccccagagacatcatgttggaagaattatcccatctcagtaaccgtggtgccaggctatttaagatgcgtcaaagaagatctgacaaatacacatttgaaaatttccagtatcaatctagagcacaaataaatcacagtattgctatgcagaatgggaaagtggatggaagtaacttggaaggtggttcgcagcaagcccccttgactcctcccaacaccccagatccacgaagccctccaaatccagacaacattgctccaggatattctggaccactgaaggaaattcctcctgaaaaattcaacaccacagctgtccctaagtactatcaatctccctgggaacaagccattagcaatgatccggagcttttagaggctttatatcctaaacttttcaagcctgaaggaaaggcagaactgcctgattacaggagctttaacagggttgccacaccatttggaggttttgaaaaagcatcaagaatggttaaatttaaagttccagattttgagctactattgctaacagatcccaggtttatgtcctttgtcaatcccctttctggcagacggtcctttaataggactcctaagggatggatatctgagaatattcctatagtgataacaaccgaacctacagatgataccactgtaccagaatcagaagacctatgaaaagaaagttgtatgtgccacataaaactctgaatataaaagttgctgttctactattttaactactggcaaagccacttgcatttttcattagtagcaacaatagcaatttagtgattttccttttctgacattcaatttcaatctcagatcaaatactaataaacaattagaaatcttactttaaaaaacttataactcacttgtcttcattcataattttgttttcacctggtttaaagaatccagatattttactgcaaaagttcagatggaaaagtaattgacagyttcacctttgtctcattttatatgatttattacagtgtaagtttttcaagtggaatctagaatcaaaatacagggagagatatgaagacctattcagagtttcatctggggatgaaagctatggaagatgatgtacaaatgttattgatggagaaaatggttggtgtgtcctttctggtgaccatgagaaatatatgtcttgatgaagtcttttcattagtcactcttagaattctaaagtgctttgcacttttncaatatgttttgaatcattaggtaatttattctgggntgatattctccaaaattcaattcagttattatattcatttaggcattaagtcaaggngact

SEQ ID NO: 18 - DNA, 886 bp, Homo sapiens
gttctcaataccgggagaggcacagagctatttcagccacatgaaaagcatcggaattgagatcgcagctcagaggacaccgggcgccccttccaccttccaaggagctttgtattcttgcatctggctgcctgggacttcccttaggcagtaaacaaatacataaagcagggataagactgcatgtatatgtcgaaacagccagtttccaatgttagagccatccaggcaaatatcaatattccaatgggagcctttcggccaggagcaggtcaaccccccagaagaaaagaatgtactcctgaagtggaggagggtgttcctcccacctcggatgaggagaagaagccaattccaggagcgaagaaacttccaggacctgcagtcaatctatcggaaatccagaatattaaaagtgaactaaaatatgtccccaaagctgaacagtagtaggaagaaaaaaggattgatgtgaagaaataaagaggcagaagatggattcaatagctcactaaaattttatatatttgtatgatgattgtgaacctcctgaatgcctgagactctagcagaaatggcctgtttgtacatttatatctcttccttctagttggctgtatttcttactttatcttcatttttggcacctcacagaacaaattagcccataaattcaacacctggagggtgtggttttgaggagggatatgattttatggagaatgatatggcaatgtgcctaacgattttgatgaaaagtttcccaagctacttcctacagtattttggtcaatatttggaatgcgttttagttcttcaccttttaaattatgtcactaaactttgtatgagttcaaataaatatttgactaaatgtaaaatgtga

SEQ ID NO: 19 - DNA, 1901 bp, Homo sapiens
aaaaaacagcagggttagcttgtccctcccctccctcttcagcttcccagacactgattctggaatgaaaattcacctgcctctgagttggctcctaatgggggtgggagtgttacttcggttcccaggttggaagattatctcacccggccccagctatataagctgaccggtgtggaggggcccagcagggccaactccagggattccttccacgacagaaaaacatacaagactccttcagccaacatgatggtactgaaagtagaggaactggtcactggaaagaagaatggcaatggggaggcaggggaattccttcctgaggatttcagagatggagagtatgaagctgctgttactttagagaagcaggaggatctgaagacacttctagcccaccctgtgaccctgggggagcaacagtggaaaagcgagaaacaacgagaggcagagctcccaaagaaaaaactagaacaaagatccaagcttgaaaatttagaagaccttgaaataatcattcaactgaagaaaaggaaaaaatacaggaaaactaaagttccagttgtaaaggaaccagaacctgaaatcattacggaacctgtggatgtgcctacgtttctgaaggctgctctggagaataaactgccagtagtagaaaaattcttgtcagacaagaacaatccagatgtttgtgatgagtataaacggacagctcttcatagagcatgcttggaaggacatttggcaattgtggagaagttaatggaagctggagcccagatcgaattccgtgatatgcttgaatccacagccatccactgggcaagccgtggaggaaacctggatgttttaaaattgttgctgaataaaggagcaaaaattagcgcccgagataagttgctcagcacagcgctgcatgtggcggtgaggactggccactatgagtgcgcggagcatcttatcgcctgtgaggcagacctcaacgccaaagacagagaaggagataccccgttgcatgatgcggtgagactgaaccgctataagatgatccgactcctgattatgtatggcgcggatctcaacatcaagaactgtgctgggaagacgccgatggatctggtgctacactggcagaatggaaccaaagcaatattcgacagcctcagagagaactcctacaagacctctcgcatagctacattctgaggcaaacgacagactcttaatcagtaaatgttcactggcattttgaaggcatggcccaggagaagagacactagccataaaatctagtttctatttatcaacgtgttgtgaagatgtacctaatgaagttttgagaaagcacagggttataggtgtttaaatttcctttagtgaaactcttatttatttttatgtattcctgtttatttatttactgccacgctactgatattcagaccttcatgatcatccatctggtgagcagagcttcatttgtatataacactttcagagccttcccacccataggtagttcttaaaccaggtgaaagagcaaagttcaagtgcctacttatgtgtcattcgctcatgtaagagtttttaagagagggctgattatcacagccctcttttctcctgaatttttaatgcagaagtttgaatgaagcaagggaaggcatgtagggacaggaaaggaaacaatggaaggaaagtgattctgtgaaaaggacagtgaagccagctattttacccccaggctggattttttttttttttttttttttttttttttttaccgagtacacagagtacccaagtgaagagaacgtcatgagtgtaagtgcaaatcagtggaaggagcggcaaactgggacatgcagaattgaatttgctcaaaaaaaaaaaaaaaa

SEQ ID NO: 20 - DNA, 197 bp, Homo sapiens
acagacgaaatgaaagtgaactggtgtcacaaagatgctcagacctcatccagtgagcatatgagaatccgggggagtgaagagatggcttggctgcacatatgtgagccgactgacaaggataaaggaaaatacacttttgagattttcgatggctaatacaaccatcaacgctcccttgacctgtccggacactg

SEQ ID NO: 21 - protein, 547 aa, Homo sapiens
VKLLLQDKEICILCQKTVYPMECLVADKQNFHKSCFRCHHCNSKLSLGNYASLHGQIYCKPHFKQLFKSKGNYDEGFGHKQHKDRWNCKNQSRSVDFIPNEEPNMCKNIAENTLVPGDRNEHLDAGNSEGQRNDLRKLGERGKLKVIWPPSKEIPKKTLPFEEELKMSKPKWPPEMTTLLSPEFKSESLLEDVRTPENKGQRQDHFPFLQPYLQSTHVCQKEDVIGIKEMKMPEGRKDEKKEGRKNVQDRPSEAEDTKSNRKSAMDLNDNNNVIVQSAEKEKNEKTNQTNGAEVLQVTNTDDEMMPENHKENLNKNNNNNYVAVSYLNNCRQKTSILEFLDLLPLSSEANDTANEYEIEKLENTSRISELLGIFESEKTYSRNVLAMALKKQTDRAAAGSPVQPAPKPSLSRGLMVKGGSSIISPDTNLLNIKGSHSKSKNLHFFFSNTVKITAFSKKNENIFNCDLIDSVDQIKNMPCLDLREFGKDVKPWHVETTEAARNNENTGFDALSHECTAKPLFPRVEVQSEQLTVEEQIKRNRCYSDTE

SEQ ID NO: 22 - DNA, 600 bp, Homo sapiens
agtcttggcggaggtgaccaaagccacgtaatgtccgtagttcgctcatccgtccatgccagatggattgtggggaaggtgattgggacaaaaatgcaaaagactgctaaagtgagagtgaccaggcttgttctggatccctatttattaaagtattttaataagcggaaaacctactttgctcacgatgcccttcagcagtgcacagttggggatattgtgcttctcagagctttacctgttccacgagcaaagcatgtgaaacatgaactggctgagatcgttttcaaagttggaaaagtcatagatccagtgacaggaaagccctgtgctggaactacctacctggagagtccgttgagttcggaaaccacccagctaagcaaaaatctggaagaactcaatatctcttcagcacagtgaagcgggagtggaagaagggtctaaagggaaaaactgacatgtttatgttatggaaaaagaaatttttctaagtttcatcacaaactgtgtccagtttctctgtggtgtttatgaaatagctaaaagcaaatgaagtaaagggcatactatggtttttcacaaaaaaaaaaaaaaaaa

SEQ ID NO: 23 - DNA, 383 bp, Homo sapiens
acccctcttctgcagacgcaggcgggtgaggatctcctcgaacttggggtgcttgctcaggtgcgccagcttcacatgcacgcctccacgcagcccagtgcccaggttggatgggcaggtgagcacgtagcccaggtgctggttccacatgaaggggtggccagctttcttaaagatctcctcaatcttctgcagccctacgcagaagcggcggaaaacctccttcatgttgccccccttctccatggagatgacccggaggtgatcctcctcgttcacccacaccaggaagctcttgttgtcattgtggcagatgccacgggcgtcgggccagtcgcgggccatgcctgaggccagcagcagcgggggacacgggctttgtc

SEQ ID NO: 24 - DNA, 403 bp, Homo sapiens
actttgagaagttactttctaattacgtcatgagaacacaacttgtaattagcaacacttctgtcagtctagatcacttcttctgcagagagcttttcaaccaagttggcatcaaccagcacaataaagttttcactgttttacctgtttcctgtatatggtgtaatcagtgaaagaaatggcatttcacatcctaaataatacggtgaaacactgtctaaaaattacttagatttaacagaattgcaattaggttttgacaatgtatttacttcaagacaatgtattttatcaggaaaaaatatcttgaaagaaagatctctgaaattatttttcatttgatacgccttttctgtgacaaaattttggggtgaaatggatgatgtttactgattgatttagt

SEQ ID NO: 25 - DNA, 168 bp, Homo sapiens
acagttccgggaggaacaagaccttcctctgctatgtggttgaagcacagggcaaggggggccaagtgcaggcatctcggggatacctagaggatgagcatgcggctgcccatgcagaggaagctttcttcaacaccatcctgccagccttcgacccagccctgcggt

SEQ ID NO: 26 - DNA, 334 bp, Homo sapiens
actatcaatctccctgggaacaagccattagcaatgatccggagcttttagaggctttatatcctaaacttttcaagcctgaaggaaaggcagaactgcctgattacaggagctttaacagggttgccacaccatttggaggttttgaaaaagcatcaagaatggttaaatttaaagttccagattttgagctactattgctaacagatcccaggtttatgtcctttgtcaatcccctttctggcagacggtcctttaataggactcctaagggatggatatctgagaatattcctatagtgataacaaccgaacctacagatgataccactgt

SEQ ID NO: 27 - DNA, 290 bp, Homo sapiens
actcctgaagtggaggagggtgttcctcccacctcggatgaggagaagaagccaattccaggagcgaagaaacttccaggacctgcagtcaatctatcggaaatccagaatattaaaagtgaactaaaatatgtccccaaagctgaacagtagtaggaagaaaaaaggattgatgtgaagaaataaagaggcagaagatggattcaatagctcactaaaattttatatatttgtatgatgattgtgaacctcctgaatgcctgagactctagcagaaatggcctgtttgt

SEQ ID NO: 28 - DNA, 152 bp, Homo sapiens
ctgattatcacagccctcttttctcctgaatttttaatgcagaagtttgaatgaagcaagggaaggcatgtagggacaggaaaggaaacaatggaaggaaagtgattctgtgaaaaggacagtgaagccagctattttacccccaggctgga

SEQ ID NO: 29 - DNA, 59 bp, Homo sapiens
tccagggattccttccacgacagaaaaacatacaagactccttcagccaacatgatggt

SEQ ID NO: 30 - DNA, 234 bp, Homo sapiens
  misc_feature 5, 26, 149, 198, 227: n = A,T,C or G
tccanggattccttccacgacagaanaacatacaagactccttcagccaacatgatggtactgaaagtagaggaactggtcactggaaagaacaatggcaatggggaggcaggggaattccttcctgaggatttcagagatggacagtntgaagctgctgttactttagagaagcaggaggatctgaagacacttctngcccaccctgtgaccctgggggagcaacngtggaaa

SEQ ID NO: 31 - DNA, 17 bp, Artificial Sequence
  T7 RNA polymerase promoter-derived sequence
acgactcactatagggc

SEQ ID NO: 32 - DNA, 16 bp, Artificial Sequence
  Primer
acaatttcacacagca

SEQ ID NO: 33 - DNA, 24 bp, Artificial Sequence
  M13 Primer
agcggataacaatttcacacagga

SEQ ID NO: 34 - DNA, 22 bp, Artificial Sequence
  T7 Primer
gtaatacgactcactatagggc

SEQ ID NO: 35 - DNA, 44 bp, Artificial Sequence
  YFP 5′ Primer
ggactagttctagagaattcctcgaggtgagcaagggcgaggag

SEQ ID NO: 36 - DNA, 20 bp, Artificial Sequence
  YFP 3′ Primer
agttggtaatggtagcgacc

SEQ ID NO: 37 - DNA, 21 bp, Artificial Sequence
  5′ Kozak oligonucleotide
ctagaactagttccaccatgg

SEQ ID NO: 38 - DNA, 21 bp, Artificial Sequence
  3′ Kozak oligonucleotide
aattccatggtggaactagtt